(19) United States
(12) Patent Application Publication
(10) Pub. No.: US 2023/0102353 A1
(43) Pub. Date: Mar. 30, 2023

(54) CHEMICALLY AMPLIFIED POSITIVE-TYPE PHOTOSENSITIVE RESIN COMPOSITION, PHOTOSENSITIVE DRY FILM, METHOD FOR PRODUCING PHOTOSENSITIVE DRY FILM, METHOD FOR PRODUCING PATTERNED RESIST FILM, METHOD FOR PRODUCING SUBSTRATE PROVIDED WITH TEMPLATE, AND METHOD FOR PRODUCING A PLATED ARTICLE

(71) Applicant: TOKYO OHKA KOGYO CO., LTD., Kawasaki-shi (JP)

(72) Inventors: Yusuke KISHIMOTO, Kawasaki-shi (JP); Akiya KAWAUE, Kawasaki-shi (JP)

(21) Appl. No.: 17/757,502

(22) PCT Filed: Dec. 1, 2020
(86) PCT No.: PCT/JP2020/044733

§ 371 Date: Jun. 16, 2022

(30) Foreign Application Priority Data
Dec. 26, 2019 (JP) 2019-237500

(51) Int. Cl. G03F 7/039

(52) U.S. Cl. CPC G03F 7/0392

(57) ABSTRACT

A chemically amplified positive-type photosensitive resin composition with which a resist pattern having a rectangular cross-sectional shape is easily formed, which has satisfactory sensitivity, and which can suppress decomposition of the acid generating agent; a photosensitive dry film having a photosensitive layer including the photosensitive resin composition; a method for producing the photosensitive dry film; a method for producing a patterned resist film using the positive-type photosensitive resin composition; a method for producing a substrate provided with a template using the positive-type photosensitive resin composition; and a method for producing a plated article using the positive-type photosensitive resin composition. The photosensitive resin composition includes an acid generating agent to generate an acid by irradiation with an active ray or radiation, a resin having alkali solubility that increases under action of an acid, and an acid diffusion suppressing agent, wherein the acid generating agent includes a non-ionic acid generating agent that generates sulfonic acid upon the irradiation, and the acid diffusion suppressing agent includes a compound having a specific structure that is decomposed by the irradiation.

### TECHNICAL FIELD

The present invention relates to a chemically amplified positive-type photosensitive resin composition, a photosensitive dry film having a photosensitive layer including the chemically amplified positive-type photosensitive resin composition, a method for producing the photosensitive dry film, a method for producing a patterned resist film using the above-described chemically amplified positive-type photosensitive resin composition, a method for producing a substrate provided with a template, and a method for producing a plated article.

### BACKGROUND ART

Photofabrication is now the mainstream of a microfabrication technique. Photofabrication is a generic term describing the technology used for manufacturing a wide variety of precision components such as semiconductor packages by applying a photoresist composition to the surface of a processing target to form a photoresist layer, patterning this photoresist layer using photolithographic techniques, and then performing chemical etching, electrolytic etching, or electroforming based mainly on electroplating, using the patterned photoresist layer (photoresist pattern) as a mask.

In recent years, high density packaging technologies have progressed in semiconductor packages along with downsizing electronic equipment, and the increase in package density has been developed on the basis of mounting multi-pin thin film in packages, miniaturizing of package size, two-dimensional packaging technologies in flip-tip systems or three-dimensional packaging technologies. In these types of high density packaging techniques, connection terminals, for example, protruding electrodes (mounting terminals) known as bumps that protrude above the package or metal posts that extend from peripheral terminals on the wafer and connect rewiring (RDL) with the mounting terminals, are disposed on the surface of the substrate with high precision.

In the photofabrication as described above, a photoresist composition is used, and chemically amplified photoresist compositions containing an acid generating agent have been known as such a photoresist composition (see Patent Documents 1, 2 and the like). In the chemically amplified photoresist composition, an acid is generated from the acid generating agent upon irradiation with radiation (exposure) and diffusion of the acid is promoted through heat treatment, to cause an acid catalytic reaction with a base resin and the like in the composition, resulting in a change to the alkali-solubility of the same.

Such chemically amplified photoresist compositions are used, for example, in formation of plated articles such as bumps, metal posts, and Cu-rewiring by a plating step. Specifically, a photoresist layer having a desired film thickness is formed on a support such as a metal substrate using a chemically amplified photoresist composition, and the photoresist layer is exposed through a predetermined mask pattern and is developed to form a photoresist pattern used as a template in which portions for forming plated articles have been selectively removed (stripped). Then, bumps or metal posts, and Cu-rewiring can be formed by embedding a conductor such as copper into the removed portions (nonresist portions) using plating, and then removing the surrounding photoresist pattern. Furthermore, the chemically amplified photoresist composition is used, for example, to form an etching mask when a substrate is etched. Specifically, a photoresist layer having a desired film thickness is formed on a substrate using a chemically amplified photoresist composition, then, only a portion corresponding to a position to be etched in the photoresist layer is exposed to via a predetermined mask pattern, and the exposed photoresist layer is developed, and thus photoresist pattern to be used as an etching mask is formed.

There is a technique of blending a photoreactive acid diffusion suppressing agent (photoreactive quencher, photodegradative base) into such a chemically amplified photoresist composition (see, Patent Document 3). The photoreactive acid diffusion suppressing agent is a salt of anion and cation, and exhibits a quenching power of trapping acid generated from an acid generating agent or the like by ion exchange reaction before exposure, and is decomposed and loses the quenching power after exposure. Consequently, when a resist film formed using a chemically amplified resist composition containing a photoreactive acid diffusion suppressing agent is subjected to exposure, the photoreactive acid diffusion suppressing agent loses the basicity with respect to acid generated from an acid generating agent or the like in exposed portions, while the photoreactive acid diffusion suppressing agent traps acid in unexposed portions. As a result, the diffusion of acid from the exposed portions to the unexposed portions can be suppressed, thereby improving lithography properties.
Patent Document 1: Japanese Unexamined Patent Application, Publication No. H9-176112
Patent Document 2: Japanese Unexamined Patent Application, Publication No. H11-52562
Patent Document 3: Japanese Unexamined Patent Application, Publication No. 2013-200560

### DISCLOSURE OF THE INVENTION

### Problems to be Solved by the Invention

In general, when a resist pattern is formed, its cross-sectional shape is desirably rectangular in many cases. For example, in formation of connection terminals such as bumps and metal posts, or formation of Cu-rewiring, by the plating as mentioned above, the cross-sectional shape of a nonresist portion of the resist pattern as a template is strongly desired to be rectangular. In the formation process of a plated article, when the cross-sectional shape of a nonresist portion of the resist pattern as a template is rectangular, a contact area between the bottom surface of the connection terminals such as bumps, metal posts, and the like, or the Cu-rewiring and a support can be sufficiently secured. Thus, connection terminals or Cu-rewiring in which adhesion to the support is good can easily be obtained.

When a resist pattern is formed using conventionally known chemically amplified resist compositions as disclosed in Patent Documents 1 and 2, a resist pattern having a rectangular cross-sectional shape is not easily formed so often. Such a problem can be solved by blending the photoreactive acid diffusion suppressing agent disclosed in Patent Document 3 into a chemically amplified positive-type photoresist composition. In this case, a resist pattern having improved lithography property and a rectangular cross-sectional shape can be easily formed.

However, when the photoreactive acid diffusion suppressing agent is blended in the chemically amplified positive-type photoresist composition, the acid generating agent is decomposed, and stability of the chemically amplified positive-type photoresist composition may be deteriorated.

Furthermore, in order to form a connection terminal, Cu-rewiring, and the like, with high accuracy, good sensitivity to irradiated radiation and the like is required such that a resist pattern is obtained with less exposure amount.

The present invention has been made in view of the above-mentioned problem, and has an object to provide a chemically amplified positive-type photosensitive resin composition with which a resist pattern having a rectangular cross-sectional shape is easily formed, which has satisfactory sensitivity, and which can suppress decomposition of the acid generating agent; a photosensitive dry film having a photosensitive layer including the chemically amplified positive-type photosensitive resin composition; a method for producing the photosensitive dry film; a method for producing a patterned resist film using the above-described chemically amplified positive-type photosensitive resin composition; a method for producing a substrate provided with a template using the above-described chemically amplified positive-type photosensitive resin composition; and a method for producing a plated article using the above-described chemically amplified positive-type photosensitive resin composition.

### Means for Solving the Problems

In order to achieve the above-mentioned objects, the present inventors have conducted extensive studies and found that the above-mentioned problem can be solved by a chemically amplified positive-type photosensitive resin composition including an acid generating agent (A) to generate an acid by irradiation with an active ray or radiation, a resin (B) having alkali solubility that increases under action of an acid, and an acid diffusion suppressing agent (C), wherein the acid generating agent (A) includes a non-ionic acid generating agent to generate sulfonic acid by irradiation with an active ray or radiation, and the acid diffusion suppressing agent (C) includes a compound decomposed by the irradiation with an active ray or radiation and represented by the following formula (c1), and the present inventors have completed the present invention. Specifically, the present invention provides the followings.

A first aspect of the present invention is a chemically amplified positive-type photosensitive resin composition including an acid generating agent (A) to generate an acid by irradiation with an active ray or radiation, a resin (B) having alkali solubility that increases under action of an acid, and an acid diffusion suppressing agent (C),

the acid generating agent (A) comprising a non-ionic acid generating agent to generate sulfonic acid by irradiation with an active ray or radiation,
the acid diffusion suppressing agent (C) comprising a compound decomposed by irradiation with an active ray or radiation, and represented by the following formula (c1):

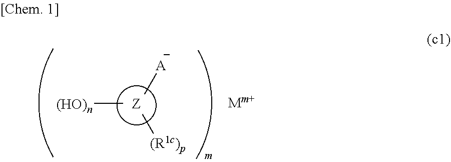

(in the formula (c1),
M^{m+} represents an m-valent organic cation,
m represents an integer of 1 or more,
a ring Z represents a benzene ring, or a benzene ring-fused polycyclic,
a number x of benzene rings of the ring Z is an integer of 1 or more and 4 or less,
R^{1c} represents a substituent,
A⁻ represents —COO⁻ or —SO₂O⁻,
n represents an integer of 2 or more and 2x+3 or less,
p represents an integer of 0 or more and 2x+3-n or less, and
when p is 2 or more, a plurality of R^{1c}s may be the same as or different from each other, and a plurality of R^{1c}s may be linked to each other to form a ring).

A second aspect of the present invention is a photosensitive dry film including a base material film and a photosensitive layer formed on the base material film, wherein the photosensitive layer includes the chemically amplified positive-type photosensitive resin composition described in the first aspect.

A third aspect of the present invention is a method for producing a photosensitive dry film, the method including: applying the chemically amplified positive-type photosensitive resin composition according to the first aspect on a base material film to form a photosensitive layer.

A fourth aspect of the present invention is a method for producing a patterned resist film, the method including: laminating a photosensitive layer including the chemically amplified positive-type photosensitive resin composition according to the first aspect on a substrate;

exposing the photosensitive layer through irradiation with an active ray or radiation in a position-selective manner; and developing the photosensitive layer after exposure.

A fifth aspect of the present invention is a method for producing a substrate provided with a template, the method including:

laminating a photosensitive layer including the chemically amplified positive-type photosensitive resin composition according to the first aspect on a substrate having a metal surface;
exposing the photosensitive layer through irradiation with an active ray or radiation in a position-selective manner; and developing the photosensitive layer after exposure to prepare a template for forming a plated article.

A sixth aspect of the present invention is a method for producing a plated article, the method including:

plating a substrate provided with a template to form a plated article in the template, the substrate provided with a template being produced by the method for producing a substrate provided with a template described in the fourth aspect.

### Effects of the Invention

The present invention can provide a chemically amplified positive-type photosensitive resin composition with which a resist pattern having a rectangular cross-sectional shape is easily formed, which has satisfactory sensitivity, and which can suppress decomposition of the acid generating agent; a photosensitive dry film having a photosensitive layer including the chemically amplified positive-type photosensitive resin composition; a method for producing the photosensitive dry film; a method for producing a patterned resist film using the above-described chemically amplified positive-type photosensitive resin composition; a method for producing a substrate provided with a template using the above-described chemically amplified positive-type photosensitive resin composition; and a method for producing a plated article using the above-described chemically amplified positive-type photosensitive resin composition.

### Preferred Mode for Carrying Out the Invention

### <<Chemically Amplified Positive-Type Photosensitive Resin Composition>>

A chemically amplified positive-type photosensitive resin composition (hereinafter, also referred to as “photosensitive resin composition”) contains an acid generating agent (A) to generate an acid by irradiation with an active ray or radiation (hereinafter, also referred to as an “acid generating agent (A)”), a resin (B) having alkali solubility that increases under action of acid (hereinafter, also referred to as a “resin (B)”), and an acid diffusion suppressing agent (C). The acid generating agent (A) includes a non-ionic acid generating agent to generate sulfonic acid by irradiation with an active ray or radiation. The acid diffusion suppressing agent (C) includes a compound decomposed by irradiation with an active ray or radiation and represented by the following formula (c1). The photosensitive resin composition may include components such as an alkali-soluble resin (D), a sulfur-containing compound (E), and an organic solvent (S) as necessary.

Hereinafter, described are essential or optional components in the photosensitive resin composition, and a method for producing the photosensitive resin composition.

### <Acid Generating Agent (A)>

An acid generating agent (A) is a compound that generates an acid upon irradiation with active rays or radiation, and a compound that directly or indirectly generates acid by light. The acid generating agent (A) includes a non-ionic acid generating agent that generates sulfonic acid upon irradiation with active rays or radiation (hereinafter, also referred to as a “non-ionic acid generating agent”). It is preferable that the non-ionic acid generating agent is included because sensitivity with respect to, for example, a g-ray (wavelength: 436 nm), an h-ray (wavelength: 405 nm), or an i-ray (wavelength: 365 nm) is achieved. The non-ionic acid generating agent can be decomposed by the acid diffusion suppressing agent. However, the decomposition of the non-ionic acid generating agent can be suppressed by using the acid diffusion suppressing agent (C) having a specific structure that is decomposed by the irradiation by active rays or radiation described later.

Examples of the non-ionic acid generating agent include sulfonium ester compounds such as an imide sulfonate compound and an oxime sulfonate compound. The imide sulfonate compound has a structure represented by >N—O—SO₂—, and is decomposed by an N—O bond by irradiation with active rays or radiation to generate sulfonic acid. The oxime sulfonate compound has a structure represented by ═N—O—SO₂—, and is decomposed by an N—O bond by irradiation with active rays or radiation to generate sulfonic acid.

Examples of the oxime sulfonate compound include a compound represented by the following formula (a1).

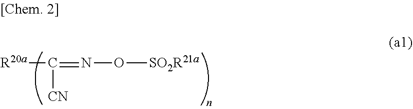

In the above formula (a1), R^{20a} represents a monovalent, bivalent or trivalent organic group, R^{21a} represents a substituted or unsubstituted saturated hydrocarbon group, an unsaturated hydrocarbon group, or an aromatic group, and n represents the number of repeating units of the structure in the parentheses.

In the formula (a1), examples of the aromatic group include aryl groups such as a phenyl group and a naphthyl group, and heteroaryl groups such as a furyl group and a thienyl group. These may have one or more appropriate substituents such as halogen atoms, alkyl groups, alkoxy groups and nitro groups on the rings. It is particularly preferable that R^{21a} is an alkyl group having 1 or more and 6 or less carbon atoms such as a methyl group, an ethyl group, a propyl group, and a butyl group. In particular, compounds in which R^{20a} represents an aromatic group, and R^{21a} represents an alkyl group having 1 or more and 4 or less carbon atoms are preferred.

Examples of the acid generating agent represented by the above formula (a1) include compounds in which R^{20a} is any one of a phenyl group, a methylphenyl group, and a methoxyphenyl group, and R^{21a} is a methyl group, when n is 1, and specific examples thereof include α-(methylsulfonyloxyimino)-1-phenylacetonitrile, α-(methylsulfonyloxyimino)-1-(p-methylphenyl)acetonitrile, α-(methylsulfonyloxyimino)-1-(p-methoxyphenyl)acetonitrile, [2-(propylsulfonyloxyimino)-2,3-dihydroxythiophene-3-ylidene](o-tolyl)acetonitrile, and the like. When n is 2, specific examples of the compound represented by the above formula (a1) include compounds represented by the following formulae.

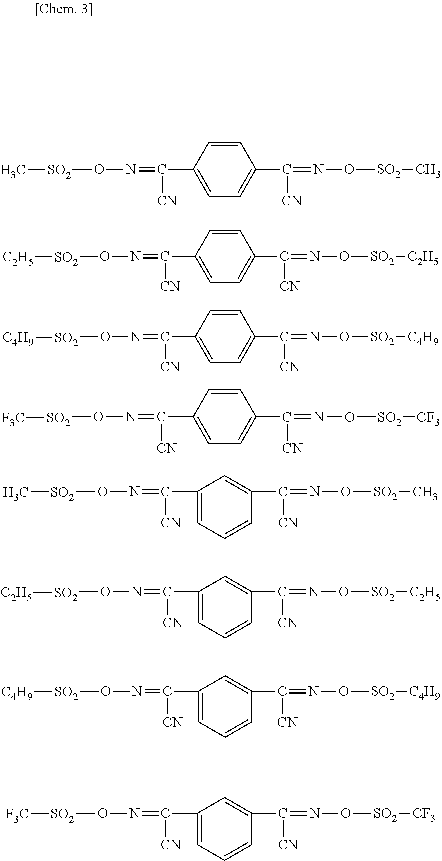

Examples of the imide sulfonate compound include a compound represented by the following formula (a2).

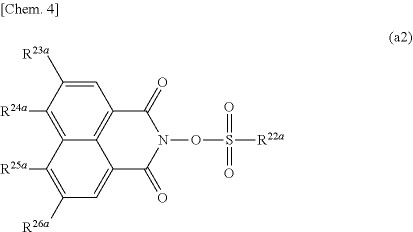

(In the formula (a2), R^{22a} is a monovalent organic group, R^{23a}, R^{24a}, R^{25a}, and R^{26a} are each independently a hydrogen atom or a monovalent organic group, each of R^{23a} and R^{24a}, R^{24a} and R^{25a}, or R^{25a} and R^{26a}, may be independently bonded to each other to form a ring.)

The organic group as R^{22a} is not particularly limited within a range in which the objects of the present invention are not impaired. The organic group may be a hydrocarbon group, and may include a heteroatom such as O, N, S, P, and a halogen atom, and the like. Furthermore, a structure of the organic group may be linear or branched or cyclic, or combination of these structures.

Examples of suitable organic group as R^{22a} include an aliphatic hydrocarbon groups having 1 or more and 18 or less carbon atoms which may be substituted with a halogen atom and/or an alkylthio group, an aryl group having 6 or more and 20 or less carbon atoms that may have a substituent, an aralkyl group having 7 or more and 20 or less carbon atoms that may have a substituent, an alkylaryl group having 7 or more and 20 or less carbon atoms that may have a substituent, a campher-10-yl group, and a group represented by the following formula (a2a).

—R^{27a}—(O)ₐ—R^{28a}—(O)_{b}—Y¹—R^{29a} (a2a)

(In the formula (a2a), Y¹ is a single bond or an alkanediyl group having 1 or more and 4 or less carbon atoms.) R^{27a} and R^{28a} are each respectively an alkanediyl group having 2 or more and 6 or less carbon atoms which may be substituted with a halogen atom, or an arylene group having 6 or more and 20 or less carbon atoms which may be substituted with a halogen atom. R^{29a} is an alkyl group having 1 or more and 18 or less carbon atoms which may be substituted with a halogen atom, an alicyclic hydrocarbon group having 3 or more and 12 or less carbon atoms, an aryl group having 6 or more and 20 or less carbon atoms which may be substituted with a halogen atom, and an aralkyl group having 7 or more and 20 or less carbon atoms that may be substituted with a halogen atom. a and b are each respectively 0 or 1, and at least one of a and b is 1.)

When an organic group as R^{22a} has a halogen atom as a substituent, examples of halogen atom include a chlorine atom, a bromine atom, an iodine atom, and a fluorine atom.

When an organic group as R^{22a} is an alkyl group having 1 or more and 18 or less carbon atoms substituted with alkylthio group, the number of carbon atoms of an alkylthio group is preferably 1 or more and 18 or less. Examples of an alkylthio group having 1 or more and 18 or less carbon atoms include a methylthio group, a ethylthio group, an n-propylthio group, an isopropylthio group, an n-butylthio group, a sec-butylthio group, a tert-butylthio group, an isobutylthio group, an n-pentylthio group, an isopentylthio group, a tert-pentylthio group, an n-hexylthio group, an n-heptylthio group, an isoheptylthio group, a tert-heptylthio group, an n-octylthio group, an isooctylthio group, a tert-octylthio group, a 2-ethylhexylthio group, an n-nonylthio group, an n-decylthio group, an undecylthio group, an n-dodecylthio group, an n-tridecylthio group, an n-tetradecylthio group, an n-pentadecylthio group, an hexadecylthio group, an n-heptadecylthio group, and an n-octadecylthio group.

When the organic group as R^{22a} is an aliphatic hydrocarbon group having 1 or more and 18 or less carbon atoms which may be substituted with a halogen atom, and/or an alkylthio group, the aliphatic hydrocarbon group may include an unsaturated double bond. Furthermore, a structure of the aliphatic hydrocarbon group is not particularly limited, and may be linear or branched or cyclic, or combination of these structures.

When the organic group as R^{22a} is an alkenyl group, suitable examples include an allyl group and a 2-methyl-2-propenyl group.

When the organic group as R^{22a} is an alkyl group, suitable examples include a methyl group, an ethyl group, an n-propyl group, an isopropyl group, an n-butyl group, a sec-butyl group, a tert-butyl group, an isobutyl group, an n-pentyl group, an isopentyl group, a tert-pentyl group, an n-hexyl group, an n-hexane-2-yl group, an n-hexane-3-yl group, an n-heptyl group, an n-heptane-2-yl group, an n-heptane-3-yl group, an isoheptyl group, a tert-heptyl group, an n-octyl group, an isooctyl group, a tert-octyl group, a 2-ethylhexyl group, an n-nonyl group, an isononyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group.

When the organic group as R^{22a} is an alicyclic hydrocarbon group, examples of the alicyclic hydrocarbon composing a main skeleton of the alicyclic hydrocarbon groups include cyclopropane, cyclobutane, cyclopentane, cyclohexane, cycloheptane, cyclooctane, cyclodecane, bicycle[2.1.1]hexane, bicycle[2.2.1]heptane, bicycle[3.2.1]octane, bicycle[2.2.2]octane, and adamantane. As an alicyclic hydrocarbon group, a group in which one hydrogen atom is removed from these alicyclic hydrocarbons is preferable.

When the organic group as R^{22a} is an aliphatic hydrocarbon group substituted with a halogen atom, suitable examples include a trifluoromethyl group, a pentafluoroethyl group, a 2-chloroethyl group, a 2-bromoethyl group, a heptafluoro-n-propyl group, a 3-bromopropyl group, a nonafluoro-n-butyl group, a tridecafluoro-n-hexyl group, a heptadecafluoro-n-octyl group, a 2,2,2-trifluoroethyl group, a 1,1-difluoroethyl group, a 1,1-difluoro-n-propyl group, a 1,1,2,2-tetrafluoro-n-propyl group, a 3,3,3-trifluoro-n-propyl group, a 2,2,3,3,3-pentafluoro-n-propyl group, a 2-norbornyl-1,1-difluoroethyl group, a 2-norborynyltetrafluoroethyl group, and a 3-adamantyl-1,1,2,2-tetrafluoropropyl group.

When the organic group as R^{22a} is an aliphatic hydrocarbon group substituted with an alkylthio group, suitable examples include 2-methylthioethyl group, 4-methylthio-n-butyl group, and 2-n-butylthioethyl group.

When the organic group as R^{22a} is an aliphatic hydrocarbon group substituted with a halogen atom and an alkylthio group, suitable examples include 3-methylthio-1,1,2,2-tetrafluoro-n-propyl group.

When the organic group as R^{22a} is an aryl group, suitable examples include a phenyl group, a naphthyl group, and a biphenylyl group.

When the organic group as R^{22a} is an aryl group substituted with a halogen atom, suitable examples include a pentafluorophenyl group, a chlorophenyl group, a dichlorophenyl group, and a trichlorophenyl group.

When the organic group as R^{22a} is an aryl group substituted with an alkylthio group, suitable examples include a 4-methylthiophenyl group, a 4-n-butylthiophenyl group, a 4-n-octylthiophenyl group, and 4-n-dodecylthiophenyl group.

When the organic group as R^{22a} is an aryl group substituted with a halogen atom and an alkylthio group, suitable example include a 1,2,5,6-tetrafluoro-4-methylthiophenyl group, a 1,2,5,6-tetrafluoro-4-n-butylthiophenyl group, and a 1,2,5,6-tetrafluoro-4-n-dodecylthiophenyl group.

When the organic group as R^{22a} is an aralkyl group, suitable examples include a benzyl group, a phenethyl group, a 2-phenylpropane-2-yl group, a diphenylmethyl group, and a triphenyl methyl group.

When the organic group as R^{22a} is an aralkyl group substituted with a halogen atom, suitable examples include a pentafluorophenylmethyl group, a phenyldifluoromethyl group, a 2-phenyltetrafluoroethyl group, and a 2-(pentafluorophenyl) ethyl group.

When the organic group as R^{22a} is an aralkyl group substituted with an alkylthio group, suitable examples include a p-methylthiobenzyl group.

When the organic group as R^{22a} is an aralkyl group substituted with a halogen atom and an alkylthio group, suitable examples include a 2-(2,3,5,6-tetrafluoro-4-methylthiophenyl) ethyl group.

When the organic group as R^{22a} is an alkylaryl group, suitable examples include a 2-methylphenyl group, a 3-methylphenyl group, a 4-methylphenyl group, a 3-isopropylphenyl group, a 4-isopropylphenyl group, a 4-n-butylphenyl group, a 4-isobutylphenyl group, a 4-tert-butylphenyl group, a 4-n-hexylphenyl group, a 4-cyclohexylphenyl group, a 4-n-octylphenyl group, a 4-(2-ethyl-n-hexyl)phenyl group, a 2,3-dimethylphenyl group, a 2,4-dimethylphenyl group, a 2,5-dimethylphenyl group, a 2,6-dimethylphenyl group, a 3,4-dimethylphenyl group, a 3,5-dimethylphenyl group, a 2,4-di-tert-butylphenyl group, a 2,5-di-tert-butylphenyl group, a 2,6-di-tert-butylphenyl group, a 2,4-di-tert-pentylphenyl group, a 2,5-di-tert-pentylphenyl group, a 2,5-di-tert-octylphenyl group, a 2-cyclohexylphenyl group, 3-cyclohexylphenyl group, 4-cyclohexylphenyl group, a 2,4,5-trimethylphenyl group, a 2,4,6-trimethylphenyl group, and 2,4,6-triisopropylphenyl group.

A group represented by the formula (a2a) is a group containing an ether group. In the formula (a2a), examples of an alkanediyl group having 1 or more and 4 or less carbon atoms represented by Y¹ include a methylene group, an ethane-1,2-diyl group, an ethane-1,1-diyl group, a propane-1,3-diyl group, a propane-1,2-diyl group, a butane-1,4-diyl group, a butane-1,3-diyl group, a butane-2,3-diyl group, and a butane-1,2-diyl group. In the formula (a2a), examples of an alkanediyl group having 2 or more and 6 or less carbon atoms represented by R^{27a} or R^{28a} include an ethane-1,2-diyl group, a propane-1,3-diyl group, a propane-1,2-diyl group, a butane-1,4-diyl group, a butane-1,3-diyl group, a butane-2,3-diyl group, a butane-1,2-diyl group, a pentane-1,5-diyl group, a pentane-1,3-diyl group, a pentane-1,4-diyl group, a pentane-2,3-diyl group, a hexane-1,6-diyl group, a hexane-1,2-diyl group, a hexane-1,3-diyl group, a hexane-1,4-diyl group, a hexane-2,5-diyl group, a hexane-2,4-diyl group, and a hexane-3,4-diyl group.

In the formula (a2a), when R^{27a} or R^{28a} is an alkanediyl group having 2 or more and 6 or less carbon atoms substituted with halogen atom(s), examples of the halogen atom include a chlorine atom, a bromine atom, an iodine atom, and a fluorine atom. Examples of alkanediyl group substituted with halogen atom(s) include a tetrafluoroethane-1,2-diyl group, a 1,1-difluoroethane-1,2-diyl group, a 1-fluoroethane-1,2-diyl group, a 1,2-difluoroethane-1,2-diyl group, a hexafluoropropane-1,3-diyl group, a 1,1,2,2-tetrafluoropropane-1,3-diyl group, and a 1,1,2,2-tetrafluoropentane-1,5-diyl group.

In the formula (a2a), examples of an arylene group as R^{27a} or R^{28a} include a 1,2-phenylene group, a 1,3-phenylene group, a 1,4-phenylene group, a 2,5-dimethyl-1,4-phenylene group, a biphenyl-4,4′-diyl group, a diphenylmethane-4,4′-diyl group, a 2,2-diphenyl propane-4,4′-diyl group, a naphthalene-1,2-diyl group, a naphthalene-1,3-diyl group, a naphthalene-1,4-diyl group, a naphthalene-1,5-diyl group, a naphthalene-1,6-diyl group, a naphthalene-1,7-diyl group, a naphthalene-1,8-diyl group, a naphthalene-2,3-diyl group, a naphthalene-2,6-diyl group, an naphthalene-2,7-diyl group.

In the formula (a2a), when R^{27a} or R^{28a} is an arylene group substituted with halogen atom(s), examples of the halogen atom include a chlorine atom, a bromine atom, an iodine atom, and a fluorine atom. Examples of an arylene group substituted with halogen atom(s) include 2,3,5,6-tetrafluoro-1,4-phenylene group.

In the formula (a2a), examples of an optionally branched alkyl group having 1 or more and 18 or less carbon atoms represented by R^{29a} include a methyl group, an ethyl group, an n-propyl group, an isopropyl group, an n-butyl group, a sec-butyl group, a tert-butyl group, an isobutyl group, an n-pentyl group, a isopentyl group, a tert-pentyl group, an n-hexyl group, an n-hexane-2-yl group, an n-hexane-3yl group, an n-heptyl group, an n-heptane-2-yl group, an n-heptane-3yl group, an isoheptyl group, a tert-heptyl group, an n-octyl group, an isooctyl group, a tert-octyl group, a 2-ethylhexyl group, an n-nonyl group, an isononyl group, an n-decyl group, an n-undecyl group, an n-dodecyl group, an n-tridecyl group, an n-tetradecyl group, an n-pentadecyl group, an n-hexadecyl group, an n-heptadecyl group, and an n-ocatadecyl group.

In the formula (a2a), when R^{29a} is an alkyl group having 1 or more and 18 or less carbon atoms substituted with halogen atom(s), examples of the halogen atom include a chlorine atom, a bromine atom, an iodine atom, and a fluorine atom. Examples of an alkyl group substituted with a halogen atom include a trifluoromethyl group, a pentafluoroethyl group, a heptafluoro-n-propyl group, a nonafluoro-n-butyl group, a tridecafluoro-n-hexyl group, a heptadecafluoro-n-octyl group, a 2,2,2-trifluoroethyl group, 1,1-difluoroethyl group, a 1,1-difluoro-n-propyl group, a 1,1,2,2-tetrafluoro-n-propyl group, a 3,3,3-trifluoro-n-propyl group, a 2,2,3,3,3-pentafluoro-n-propyl group, and a 1,1,2,2-tetrafluorotetradecyl group.

In the formula (a2a), when R^{29a} is an alicyclic hydrocarbon group having 3 or more and 12 or less carbon atoms, examples of the alicyclic hydrocarbon composing a main skeleton of the alicyclic hydrocarbon groups include cyclopropane, cyclobutane, cyclopentane, cyclohexane, cycloheptane, cyclooctane, cyclodecane, bicycle[2.1.1]hexane, bicycle[2.2.1]heptane, bicycle[3.2.1]octane, bicycle[2.2.2]octane, and adamantane. As an alicyclic hydrocarbon group, a group in which one hydrogen atom is removed from these alicyclic hydrocarbons is preferable.

In the formula (a2a), when R^{29a} is an aryl group, a halogenated aryl group, an aralkyl group, and a halogenated aralkyl group, preferable examples of these groups are the same as the case where R^{22a} are these groups.

The suitable group among the groups represented by the formula (a2a) is a group among the groups represented by R^{27a} in which a carbon atom bonded to a sulfur atom is substituted with a fluorine atom. The number of carbon atoms of such a suitable group is preferably 2 or more and 18 or less.

R^{22a} is preferably a perfluoroalkyl group having 1 or more and 8 or less carbon atoms. Furthermore, since highly minute resist patterns are easily formed, a camphor-10-yl group is also preferable as R^{22a}.

In the formula (a2), R^{23a} to R^{26a} are a hydrogen atom or a monovalent organic group. Furthermore, R^{23a} and R^{24a}, R^{24a} and R^{25a}, or R^{25a} and R^{26a} may be bonded to each other respectively to form a ring. For example, R^{25a} and R^{26a} may be bonded to each other to form a five-membered ring together with a naphthalene ring, thereby forming an acenaphthene skeleton.

Preferable examples of the monovalent organic group include an alkyl group and an alkoxy group having 4 or more and 18 or less carbon atoms which may be substituted with an alicyclic hydrocarbon group, a heterocyclic group (heterocyclyl group), or a halogen atom, and which may be branched; a heterocyclyloxy group; an alkylthio group having 4 or more and 18 or less carbon atoms which may be substituted with an alicyclic hydrocarbon group, a heterocyclic group (heterocyclyl group), or a halogen atom and which may be branched; and a heterocyclylthio group. Furthermore, a group in which the methylene group at any position that is not adjacent to an oxygen atom of the alkoxy group is substituted with —CO— is also preferable. A group in which the alkoxy group is interrupted by an —O—CO— bond, or an —O—CO—NH-bond is also preferable. Note here that the left ends of the —O—CO-bond and the —O—CO—NH— bond are sides near the naphthalic acid mother nucleus in an alkoxy group. In addition, an alkylthio group having 4 or more and 18 or less carbon atoms, which may be substituted with an alicyclic hydrocarbon group, a heterocyclic group, or a halogen atom, and which may be branched, is also preferable as R^{23a} to R^{26a}. A group in which the methylene group at any position that is not adjacent to a sulfur atom of the alkylthio group is substituted with —CO— is also preferable. A group in which the alkylthio group is interrupted by an —O—CO— bond, or an —O—CO—NH— bond is also preferable. Note here that the left ends of the —O—CO— bond and —O—CO—NH— bond are sides near the naphthalic acid mother nucleus in an alkylthio group.

As R^{23a} to R^{26a}, it is preferable that R^{23a} is an organic group, R^{24a} to R^{26a} are a hydrogen atom, or R^{24a} is an organic group, and R^{23a}, R^{25a}, and R^{26a} are a hydrogen atom. Furthermore, all of R^{23a} to R^{26a} may be a hydrogen atom.

Examples of an unsubstituted alkyl group as R^{23a} to R^{26a} includes an n-butyl group, a sec-butyl group, a tert-butyl group, an isobutyl group, an n-pentyl group, an isopentyl group, a tert-pentyl group, an n-hexyl group, an n-heptyl group, an isoheptyl group, a tert-heptyl group, an n-octyl group, an isooctyl group, a tert-octyl group, a 2-ethylhexyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, and an n-ocatdecyl group.

Examples of an unsubstituted alkoxy group as R^{23a} to R^{26a} includes an n-butyloxy group, a sec-butyloxy group, a tert-butyloxy group, an isobutyloxy group, an n-pentyloxy group, an isopentyloxy group, a tert-pentyloxy group, an n-hexyloxy group, an n-heptyloxy group, an isoheptyloxy group, a tert-heptyloxy group, an n-octyloxy group, an isooctyloxy group, a tert-octyloxy group, a 2-ethylhexyloxy group, an n-nonyloxy group, an n-decyloxy group, an n-undecyloxy group, an n-dodecyloxy group, an n-tridecyloxy group, an n-tetradecyloxy group, an n-pentadecyloxy group, an n-hexadecyloxy group, an n-heptadecyloxy group, and an n-ocatdecyloxy group.

Examples of an unsubstituted alkylthio group as R^{23a} to R^{26a} includes an n-butylthio group, a sec-butylthio group, a tert-butylthio group, an isobutylthio group, an n-pentylthio group, an isopentylthio group, a tert-pentylthio group, an n-hexylthio group, an n-heptylthio group, an isoheptylthio group, a tert-heptylthio group, an n-octylthio group, an isooctylthio group, a tert-octylthio group, a 2-ethylhexylthio group, an n-nonylthio group, an n-decylthio group, an n-undecylthio group, an n-dodecylthio group, an n-tridecylthio group, an n-tetradecylthio group, an n-pentadecylthio group, an n-hexadecylthio group, an n-heptadecylthio group, and an n-ocatdecylthio group.

When R^{23a} to R^{26a} are an alkyl group, an alkoxy group or an alkylthio group substituted with an alicyclic hydrocarbon group, examples of the alicyclic hydrocarbon composing a main skeleton of the alicyclic hydrocarbon group include cyclopropane, cyclobutane, cyclopentane, cyclohexane, cycloheptane, cyclooctane, cyclodecane, bicycle[2.1.1]hexane, bicycle[2.2.1]heptane, bicycle[3.2.1]octane, bicycle[2.2.2]octane, and adamantine. As an alicyclic hydrocarbon group, a group in which one hydrogen atom is removed from these alicyclic hydrocarbons is preferable.

When R^{23a} to R^{26a} are an alkyl group, an alkoxy group or an alkylthio group substituted with a heterocyclic group, or when R^{23a} to R^{26a} are a heterocyclyloxy group, examples of heterocycle composing a main skeleton of the heterocyclic group or the heterocyclyloxy group include pyrrole, thiophene, furan, pyrane, thiopyrane, imidazole, pyrazole, triazole, isothiazole, oxazole, isoxazole, pyridine, pyrazine, pyrimidine, pyridazine, pyrrolidine, pyrazolidine, imidazolidine, isoxazolidine, isothiazolidine, piperidine, piperazine, morpholine, thiomorpholine, chroman, thiochroman, isochroman, isothiochroman, indoline, isoindoline, pyrindine, indolizine, indole, indazole, purine, quinolizine, isoquinoline, quinoline, naphthyridine, phthalazine, quinoxaline, quinazoline, cinnoline, pteridine, acridine, perimidine, phenanthroline, carbazole, carboline, phenazine, anthyridine, thiadiazole, oxadiazole, triazine, triazole, tetrazole, benzimidazole, benzoxazole, benzothiazole, benzothiadiazole, benzofuran, naphthoimidazole, benzotriazole, and tetraazaindene. Among these heterocycles, saturated heterocycles to which rings including conjugated bond(s) are hydrogenated are also preferable. A group in which one hydrogen atom is removed from above-mentioned heterocycle is preferable as a heterocyclic group substituting the alkyl group, the alkoxy group or the alkylthio group or a heterocyclic group included in the heterocyclyloxy group.

Examples of an alkoxy group containing an alicyclic hydrocarbon group as R^{23a} to R^{26a} include a cyclopentyloxy group, a methylcyclopentyloxy group, a cyclohexyloxy group, a fluorocyclohexyloxy group, a chlorocyclohexyloxy group, a cyclohexylmethyloxy group, a methylcyclohexyloxy group, a norbornyloxy group, an ethylcyclohexyloxy group, a cyclohexylethyloxy group, a dimethylcyclohexyloxy group, a methylcyclohexylnethyloxy group, a norbornylmethyloxy group, a trimethylcyclohexyloxy group, a 1-cyclohexylbutyloxy group, an adamantyloxy group, a menthyloxy group, an n-butylcyclohexyloxy group, a tert-butylcyclohexyloxy group, a bornyloxy group, an isobornyloxy group, a decahydronaphthyloxy group, a dicyclopentadienoxy group, a 1-cyclohexylpentyloxy group, a methyladamantyloxy group, an adamantymethyloxy group, a 4-pentylcyclohexyloxy group, a cyclohexylcyclohexyloxy group, an adamantylethyloxy group, and a dimethyladamantyloxy group.

Examples of a heterocyclyloxy group as R^{23a} to R^{26a} include a tetrahydrofuranyloxy group, a furfuryloxy group, a tetrahydrofurfuryloxy group, a tetrahydropyranyloxy group, a butyrolactonyl oxy group, and an indolyloxy group.

Examples of an alkylthio group including an alicyclic hydrocarbon group as R^{23a} to R^{26a} include a cyclopentylthio group, a cyclohexylthio group, a cyclohexylmethylthio group, a norbornylthio group, and an isobornylthio group.

Examples of a heterocyclylthio group as R^{23a} to R^{26a} include a furfurylthio group, and a tetrahydrofuranylthio group.

When R^{23a} to R^{26a} are a group in which a methylene group at any positon not being adjacent to oxygen atom of an alkoxy group is substituted with —CO—, examples thereof include 2-ketobutyl-1-oxy group, 2-ketopentyl-1-oxy group, 2-ketohexyl-1-oxy group, 2-ketoheptyl-1-oxy group, 2-ketooctyl-1-oxy group, 3-ketobutyl-1-oxy group, 4-ketopentyl-1-oxy group, 5-ketohexyl-1-oxy group, 6-ketoheptyl-1-oxy group, 7-ketooctyl-1-oxy group, 3-methyl-2-ketopentane-4-oxy group, 2-ketopentane-4-oxy group, 2-methyl-2-ketopentane-4-oxy group, 3-ketoheptane-S-oxy group, and 2-adamantanone-S-oxy group.

When R^{23a} to R^{26a} are a group in which a methylene group at any position that is not adjacent to sulfur atom of an alkylthio group is substituted with —CO—, examples thereof include 2-ketobutyl-1-thio group, 2-ketopentyl-1-thio group, 2-ketohexyl-1-thio group, 2-ketoheptyl-1-thio group, 2 ketooctyl-1-thio group, 3-ketobutyl-1-thio group, 4-ketopentyl-1-thio group, 5-ketohexyl-1-thio group, 6-ketoheptyl-1-thio group, 7-ketooctyl-1-thio group, 3-methyl-2-ketopentane-4-thio group, 2-ketopentane-4-thio group, 2-methyl-2-ketopentane-4-thio group, and 3-ketoheptane-S-thio group.

Specific examples of the compound represented by the formula (a2) include the following compounds.

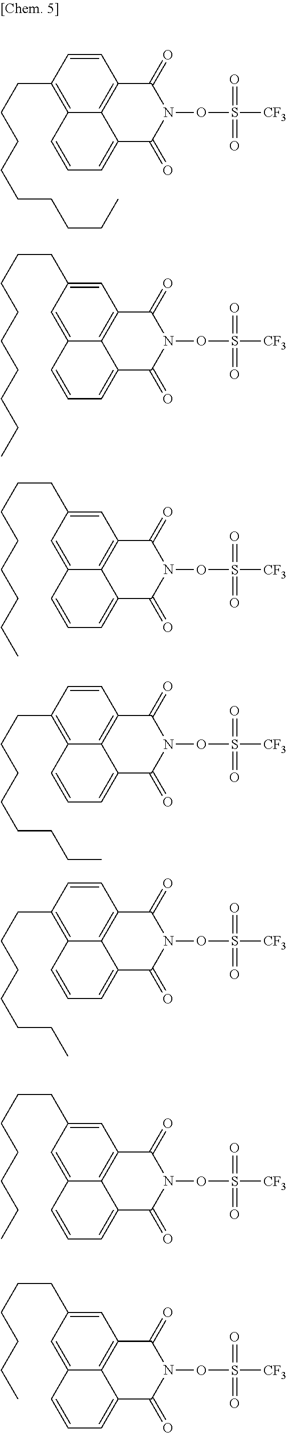

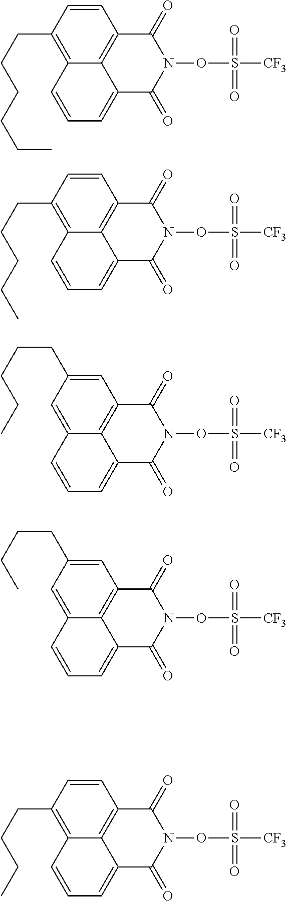

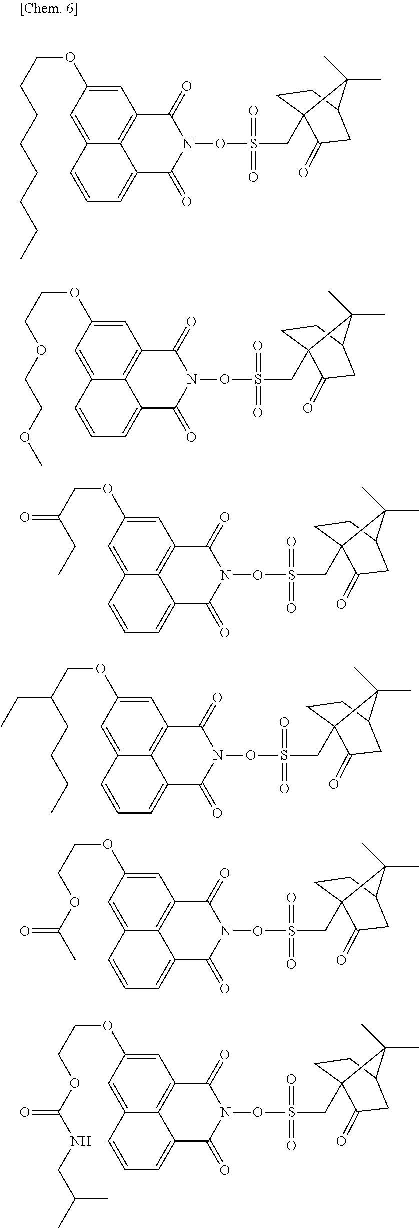

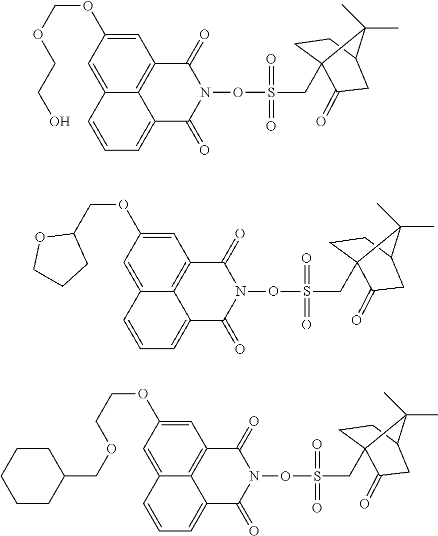

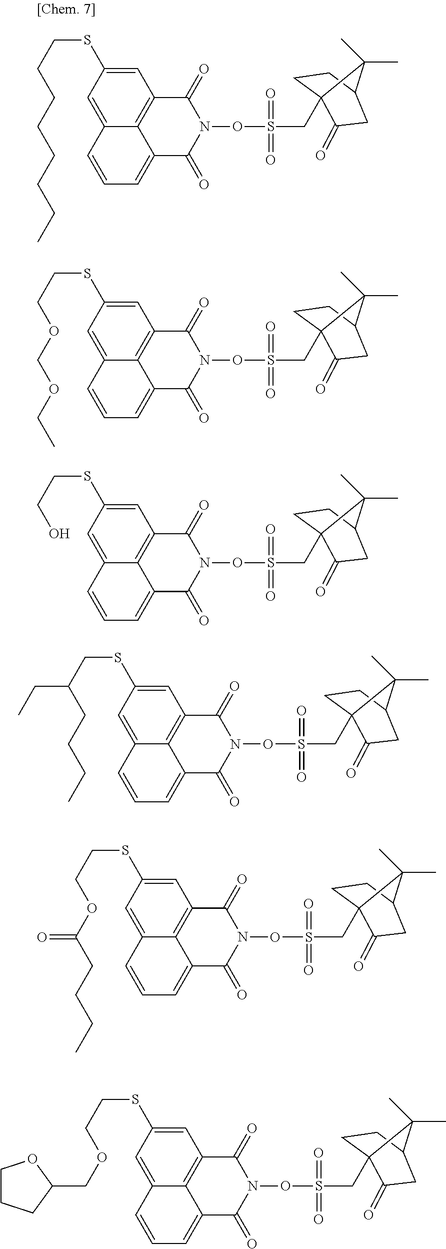

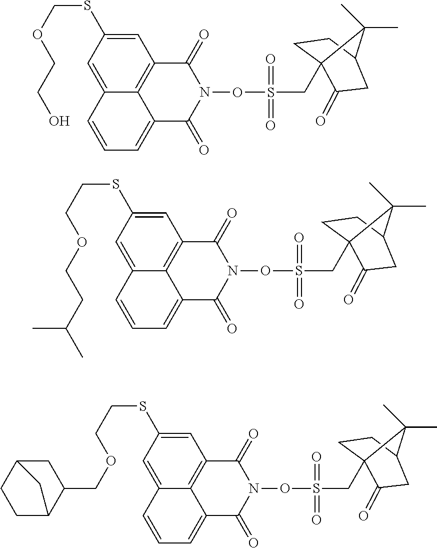

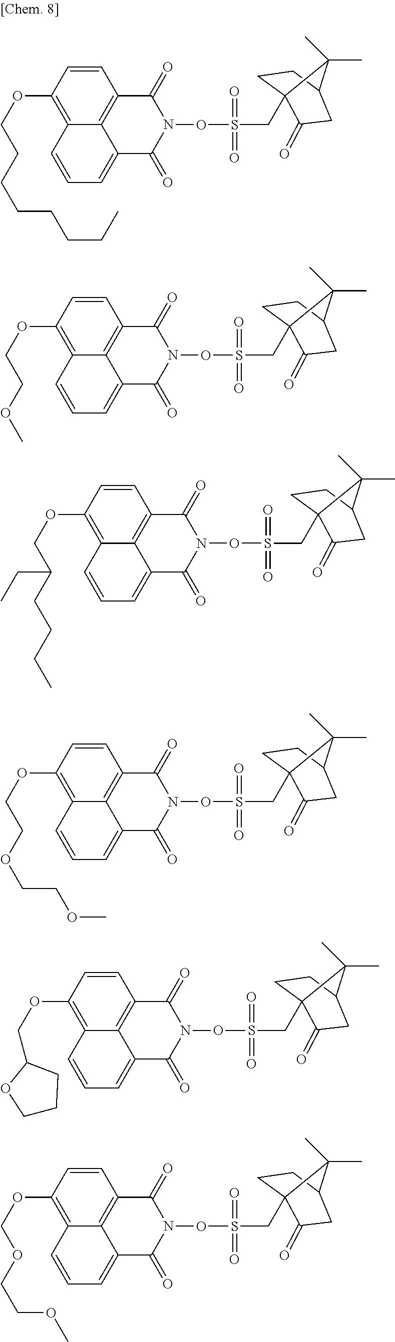

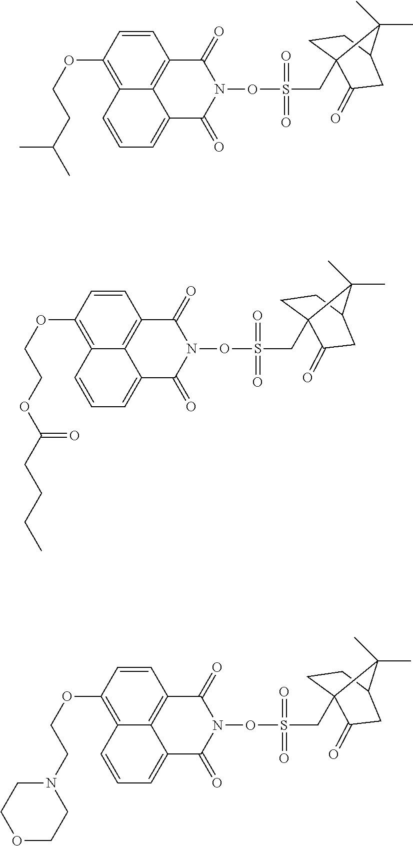 [SMILES: CC(C)CCOc1ccc2c3c(cccc13)C(=O)N(OS(=O)(=O)CC13CCC(CC1=O)C3(C)C)C2=O ; CCCCC(=O)OCCOc1ccc2c3c(cccc13)C(=O)N(OS(=O)(=O)CC13CCC(CC1=O)C3(C)C)C2=O ; CC1(C)C2CCC1(CS(=O)(=O)ON1C(=O)c3cccc4c(OCCN5CCOCC5)ccc(c34)C1=O)C(=O)C2]

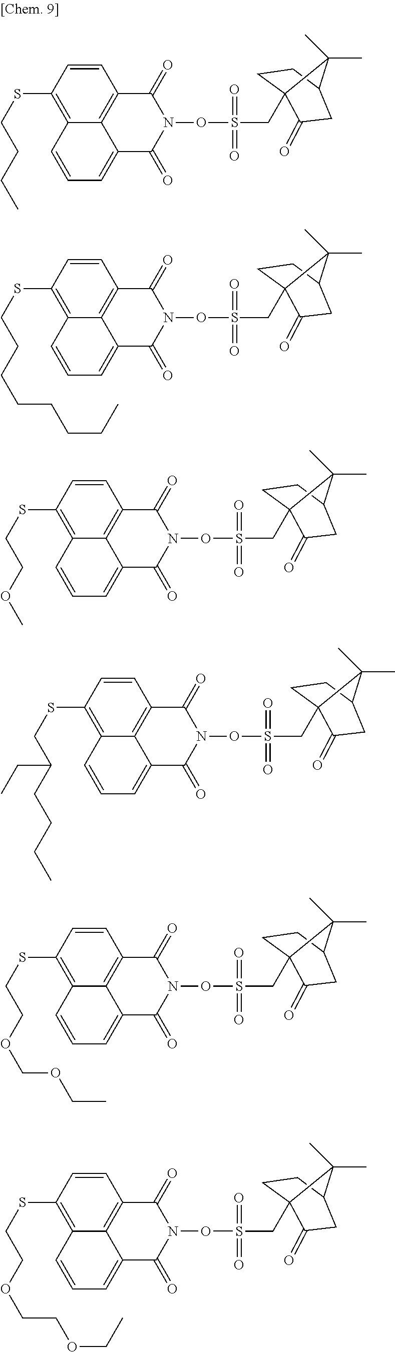

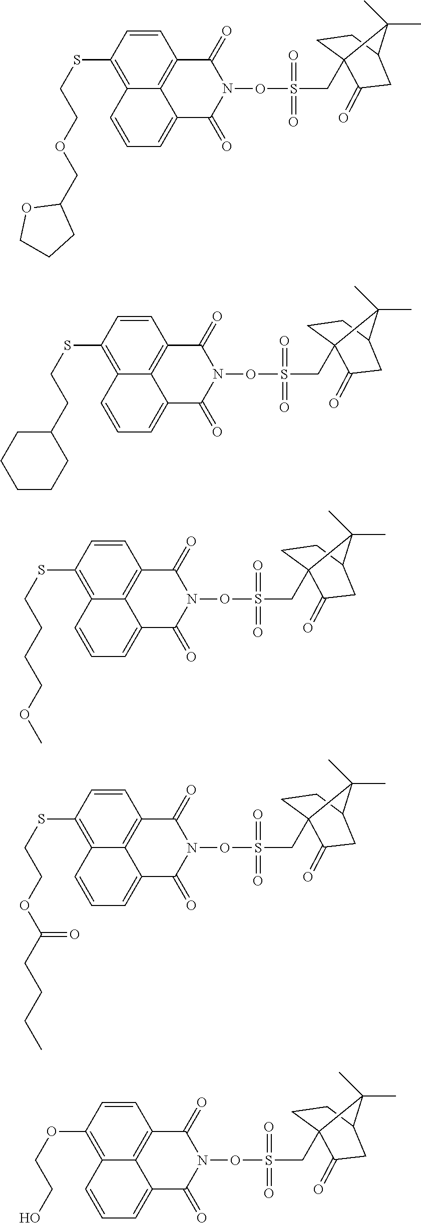 [SMILES: CC1(C)C2CCC1(CS(=O)(=O)ON1C(=O)c3cccc4c(SCCOCC5CCCO5)ccc(c34)C1=O)C(=O)C2 ; CC1(C)C2CCC1(CS(=O)(=O)ON1C(=O)c3cccc4c(SCCC5CCCCC5)ccc(c34)C1=O)C(=O)C2 ; COCCCCSc1ccc2c3c(cccc13)C(=O)N(OS(=O)(=O)CC13CCC(CC1=O)C3(C)C)C2=O ; CCCCC(=O)OCCSc1ccc2c3c(cccc13)C(=O)N(OS(=O)(=O)CC13CCC(CC1=O)C3(C)C)C2=O ; CC1(C)C2CCC1(CS(=O)(=O)ON1C(=O)c3cccc4c(OCCO)ccc(c34)C1=O)C(=O)C2]

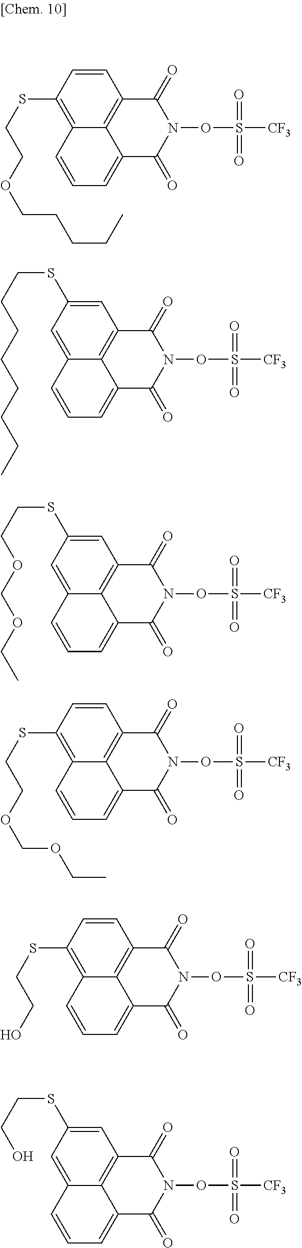

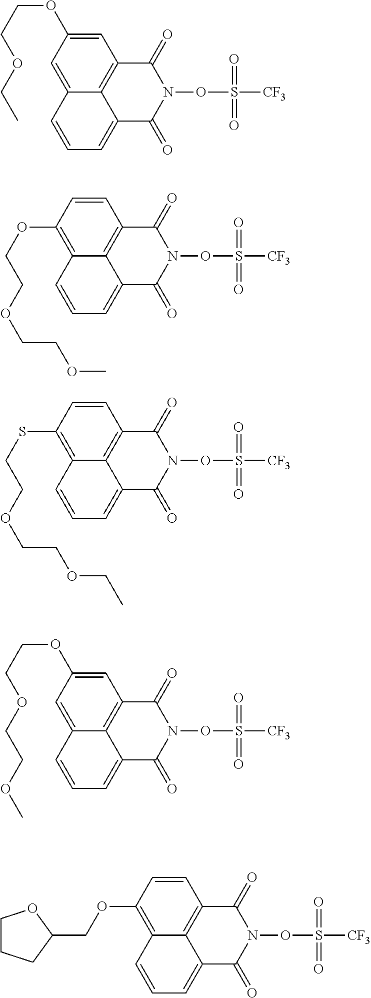

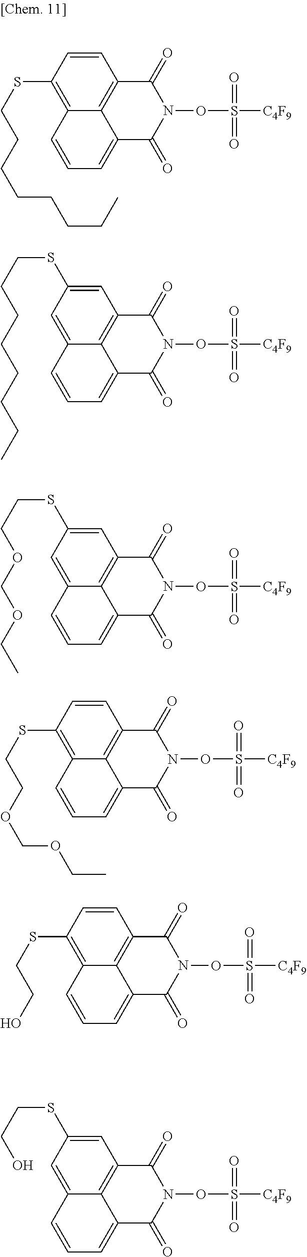

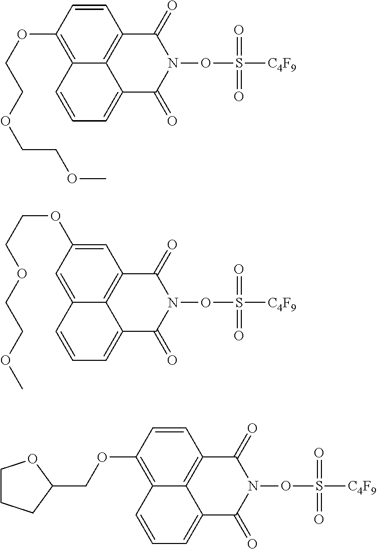

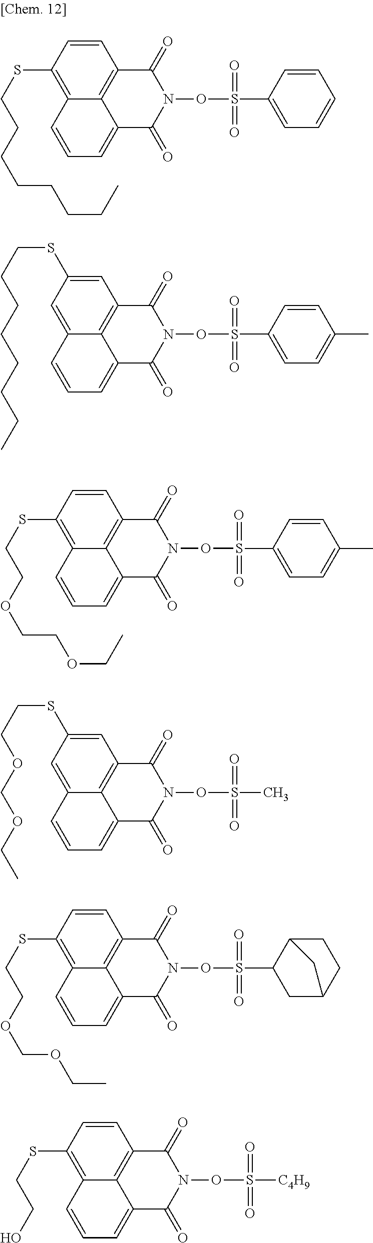

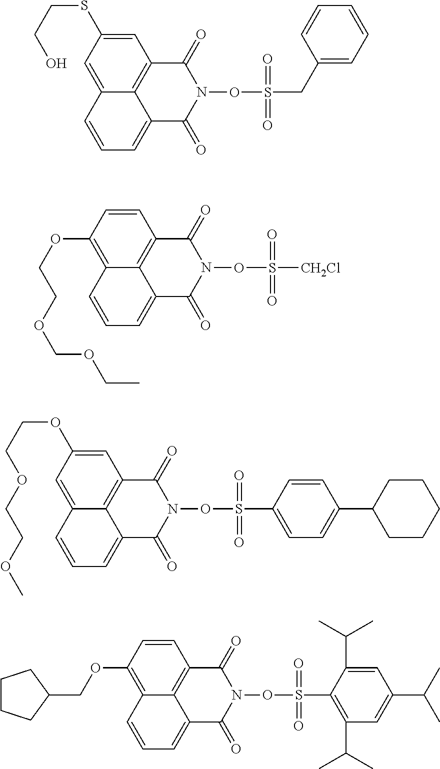

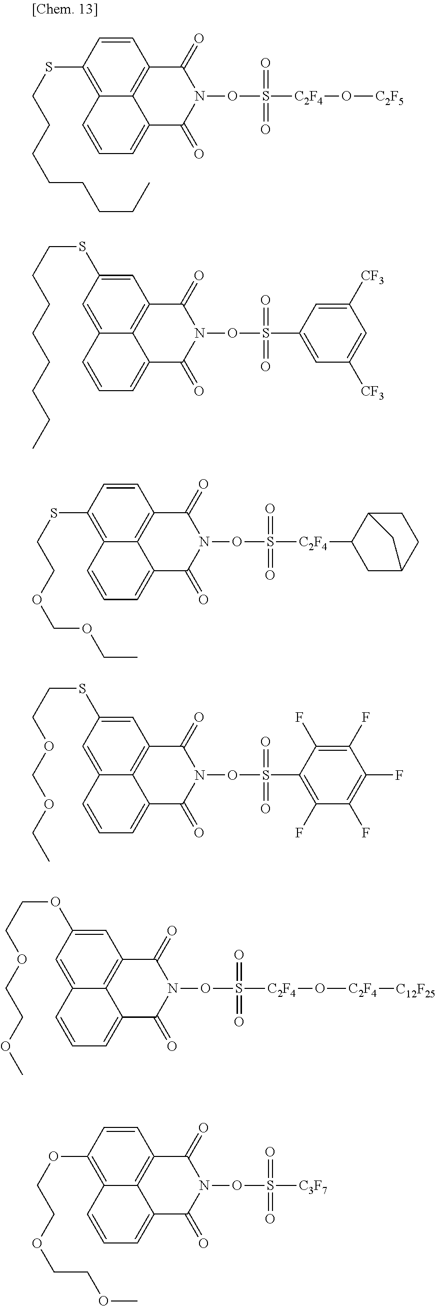

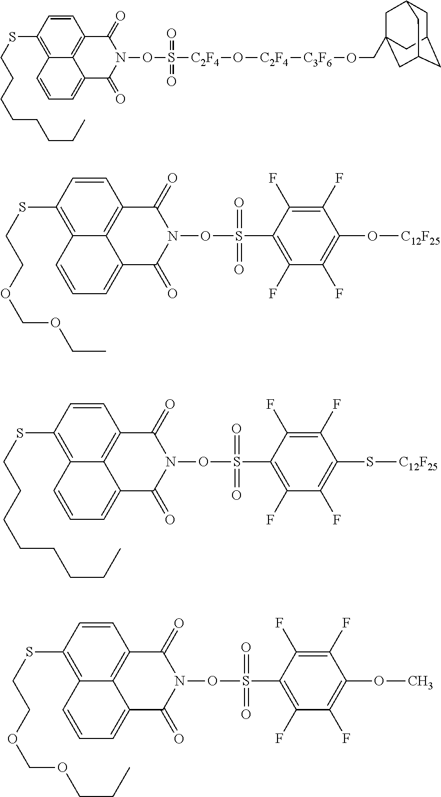

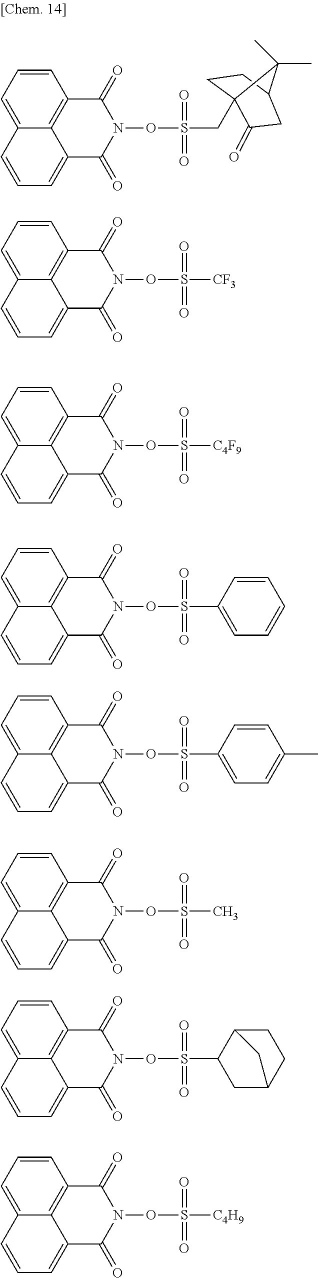

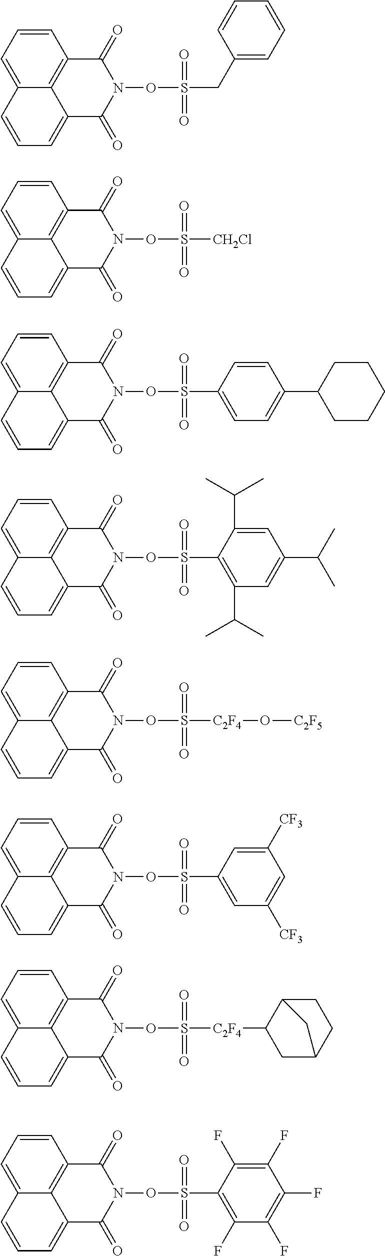

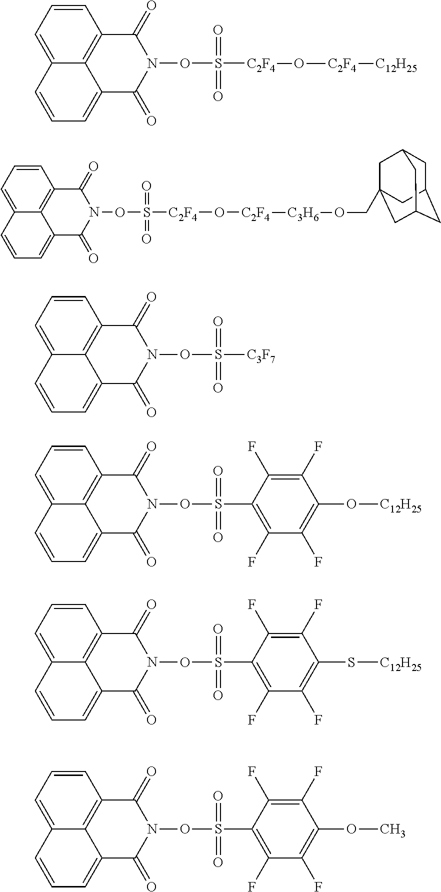

Examples of the imide sulfonate compound include also the compound represented by the following formula (a3).

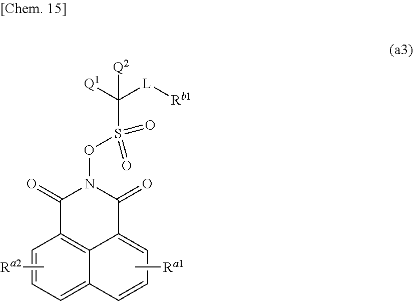

In the formula (a3), R^{b1} is a hydrocarbon group having 1 or more and 30 or less carbon atoms. When the hydrocarbon group as R^{b1} includes one or more methylene groups, at least a part of the methylene groups may be substituted with a group selected from the group consisting of —O—, —S—, —CO—, —CO—O—, —SO—, —SO₂—, —CR^{b4}R^{b5}—, and —NR^{b6}—. When the hydrocarbon group as R^{b1} includes a hydrocarbon ring, at least one of the carbon atoms constituting the hydrocarbon ring may be substituted with a hetero atom selected from the group consisting of N, O, P, S, and Se, or an atomic group including the hetero atom. R^{b4} and R^{b5} each independently is a hydrogen atom, or a halogen atom, and at least one of R^{b4} and R^{b5} is a halogen atom. R^{b6} is a hydrogen atom, or a hydrocarbon group having 1 or more and 6 or less carbon atoms. R^{a1} and R^{a2} each independently is a hydrogen atom, an aliphatic hydrocarbon group having 1 or more and 20 or less carbon atoms that may have a substituent, an aromatic group having 5 or more and 20 or less ring-constituting atoms that may have a substituent, or groups represented by —R^{a3}—R^{a4}. R^{a1} and R^{a2} are not simultaneously a hydrogen atom. When the aliphatic hydrocarbon group as R^{a1} or R^{a2} include 1 or more methylene groups, at least a part of the methylene groups may be substituted with a group selected from the group consisting of —O—, —S—, —CO—, —CO—O—, —SO—, —SO₂—, and —NR^{a5}—. R^{a5} is a hydrogen atom, or a hydrocarbon group having 1 or more and 6 or less carbon atoms. Rai is a methylene group, —O—, —CO—, —CO—O—, —SO—, —SO₂—, or —NR^{a6}—. R^{a6} is a hydrogen atom, or a hydrocarbon group having 1 or more and 6 or less carbon atoms. Rao is an aromatic group having 5 or more and 20 or less ring-constituting atoms that may have a substituent, a perfluoroalkyl group having 1 or more and 6 or less carbon atoms, an aralkyl group having 7 or more and 20 or less carbon atoms that may have a substituent, or a heteroaryl alkyl group including an aromatic heterocyclic group having 5 or more and 20 or less ring-constituting atoms that may have a substituent. Q¹ and Q² each independently is a fluorine atom, or a perfluoroalkyl group having 1 or more and 6 or less carbon atoms. L is an ester bond.

In the formula (a3), the aliphatic hydrocarbon group having 1 or more and 20 or less carbon atoms as R^{a1} and R^{a2} may be linear or branched or cyclic, or combination of these structures. As the aliphatic hydrocarbon group, an alkyl group is preferable. Suitable examples of the alkyl group include a methyl group, an ethyl group, an n-propyl group, an isopropyl group, an n-butyl group, an isobutyl group, a sec-butyl group, a tert-butyl group, an n-pentyl group, an n-hexyl group, an n-heptyl group, an n-octyl group, a 2-ethyl hexyl group, an n-nonyl group, and an n-decyl group. Examples of the substituent which the aliphatic hydrocarbon group having 1 or more and 20 or less carbon atoms as R^{a1} and R^{a2} may have include a hydroxyl group, a mercapto group, an amino group, a halogen atom, an oxygen atom, a nitro group, a cyano group, and the like. The number of substituents is arbitrary. Examples of the aliphatic hydrocarbon group as R^{a1} and R^{a2} having 1 or more and 20 or less carbon atoms and having a substituent include, for example, a perfluoroalkyl group having 1 or more and 6 or less carbon atoms. Specific examples thereof include CF₃—, CF₃CF₂—, (CF₃)₂CF—, CF₃CF₂CF₂—, CF₃CF₂CF₂CF₂—, (CF₃)₂CFCF₂—, CF₃CF₂ (CF₃) CF—, and (CF₃)₃C—.

In the formula (a3), an aromatic group having 5 or more and 20 or less ring-constituting atoms as the R^{a1} and R^{a2} which may have a substituent may be an aromatic hydrocarbon group or an aromatic heterocyclic group. Examples of the aromatic group include aryl groups such as a phenyl group and a naphthyl group, and heteroaryl groups such as a furyl group and a thienyl group. Examples of the substituents which the aromatic group having 5 or more and 20 or less ring-constituting atoms may have are the same as the substituents which the aliphatic hydrocarbon group having 1 or more and 20 or less carbon atoms as R^{a1} and R^{a2} may have.

In the formula (a3), an aromatic group having 5 or more and 20 or less ring-constituting atoms that may have a substituent as Rao is the same as the aromatic group having 5 or more and 20 or less ring-constituting atoms that may have a substituent described for R^{a1} and R^{a2}. In the formula (a3), the perfluoroalkyl group having 1 or more and 6 or less carbon atoms as Rao is the same as the perfluoroalkyl group having 1 or more and 6 or less carbon atoms described for R^{a1} and R^{a2}. In the formula (a3), specific examples of the aralkyl group having 7 or more and 20 or less carbon atoms that may have a substituent as Rao include a benzyl group, a phenethyl group, an α-naphthyl methyl group, a β-naphthyl methyl group, a 2-α-naphthyl ethyl group, a 2-β-naphthyl ethyl group, and the like. In the formula (a3), the heteroaryl alkyl group is a group in which a part of carbon atoms constituting the aromatic hydrocarbon in the arylalkyl group is substituted with hetero atoms such as N, O, S, or the like. Specific examples of the heteroaryl alkyl group including an aromatic heterocyclic group having 5 or more and 20 or less ring-constituting atoms that may have a substituent as Rao include a pyridine-2-ylmethyl group, a pyridine-3-ylmethyl group, a pyridine-4-ylmethyl group, and the like.

In the formula (a3), the hydrocarbon group having 1 or more and 6 or less carbon atoms as R^{a5} may be an aliphatic hydrocarbon group, or an aromatic hydrocarbon group, or combination of these groups. The aliphatic hydrocarbon group may be linear or branched or cyclic, or combination of these structures. Examples of the aliphatic hydrocarbon group include alkyl groups such as a methyl group, an ethyl group, an n-propyl group, an isopropyl group, an n-butyl group, an isobutyl group, a sec-butyl group, a tert-butyl group, an n-pentyl group, and an n-hexyl group. Examples of the aromatic hydrocarbon group include a phenyl group.

In the formula (a3), the hydrocarbon group having 1 or more and 6 or less carbon atoms as R^{a6} is the same as the hydrocarbon group having 1 or more and 6 or less carbon atoms described for R^{a5}.

In the formula (a3), the hydrocarbon group having 1 or more and 30 or less carbon atoms as RID′ may be an aliphatic hydrocarbon group, or an aromatic hydrocarbon group, or combination of these groups. The aliphatic hydrocarbon group may be linear or branched or cyclic, or combination of these structures. Examples of the aliphatic hydrocarbon group include chain aliphatic hydrocarbon groups such as a methyl group, an ethyl group, an n-propyl group, an isopropyl group, an n-butyl group, an isobutyl group, a sec-butyl group, a tert-butyl group, an n-pentyl group, an n-hexyl group, and cyclic aliphatic hydrocarbon groups (hydrocarbon rings) such as cyclopropyl group, a cyclobutyl group, a cyclopentyl group, cyclohexyl group, an adamantyl group, and a norbornyl group. Examples of the aromatic hydrocarbon group include a phenyl group, and a naphthyl group. Examples of a group combining the aliphatic hydrocarbon group and the aromatic hydrocarbon group include a benzyl group, a phenethyl group, and a furyl methyl group. When the hydrocarbon group as RID′ includes a hydrocarbon ring, examples of the atomic group including a hetero atom substituting at least one of carbon atoms constituting the hydrocarbon ring include —CO—, —CO—O—, —SO—, —SO₂—, —SO₂—O—, —P(═O)—(OR^{b7})₃. R^{b7} is a hydrocarbon group having 1 or more and 6 or less carbon atoms, and is the same as a hydrocarbon group having 1 or more and 6 or less carbon atoms described for R^{a5}.

In the formula (a3), specific examples of the halogen atom as R^{b4} and R^{b5} include a chlorine atom, a fluorine atom, a bromine atom, and an iodine atom.

In the formula (a3), the hydrocarbon group having 1 or more and 6 or less carbon atoms as R^{b6} is the same as the hydrocarbon group having 1 or more and 6 or less carbon atoms described for R^{a5} in the formula (a3).

In the formula (a3), the perfluoroalkyl group having 1 or more and 6 or less carbon atoms as Q¹ and Q² is the same as the perfluoroalkyl group having 1 or more and 6 or less carbon atoms described for R^{a1} and R^{a2} in the formula (a3).

In a compound represented by the formula (a3), the direction of the ester bond as L is not particularly limited, and may be —CO—O— or may be —O—CO—.

The compound represented by the formula (a3) is preferably a compound represented by the following formula (a3-1).

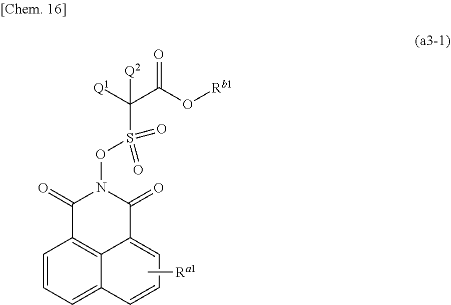

R^{b1}, R^{a1}, Q¹, and Q² in the formula (a3-1) are the same as those in the formula (a3).)

When R^{a1} in the formula (a3-1) is an aliphatic hydrocarbon group having 1 or more and 20 or less carbon atoms that may have a substituent and when the aliphatic hydrocarbon group as R^{a1} includes 1 or more methylene groups, preferable compounds include compounds represented by the formula (a3-1) are preferable in which at least a part of the methylene groups may be substituted with a group selected from the group consisting of —O—, —S—, —CO—, —CO—O—, —SO—, —SO²—, and —NR^{a5}—.

The compound represented by the formula (a3) can be produced by the following method for producing an N-organosulfonyloxy compound. The method for producing an N-organosulfonyloxy compound capable of producing a compound represented by formula (a3) is a method for producing an N-organosulfonyloxy compound including reacting an N-hydroxy compound (a′) with a sulfonic acid fluoride compound (b′) in the presence of a basic compound (d′), wherein when the N-hydroxy compound (a′) is reacted with the sulfonic acid fluoride compound (b′), a silylating agent (c′) is present in the system, and the sulfonic acid fluoride compound (b′) is represented by the following formula (b1-1), a silylating agent (c′) can convert a hydroxy group on a nitrogen atom of the N-hydroxy compound (a′) into a silyloxy group represented by formula (ac1).

—O—Si(R^{c1})₃ (ac1)

(In the formula (ac1), R^{c1} each independently is a hydrocarbon group having 1 or more and 10 or less carbon atoms.)

R^{b1}-L-CQ¹Q²-SO₂—F (b1-1)

(In the formula (b1-1), R^{b1}, L, Q¹, and Q² are respectively the same as those in in the above formula (a3).)

Furthermore, the method for producing an N-organosulfonyloxy compound capable of producing the compound represented by the formula (a3) includes: a silylation step of silylating the N-hydroxy compound (a′) with the silylating agent (c′), and a condensation step of condensing the silylated product of the N-hydroxy compound (a′) produced in the silylation step with the sulfonic acid fluoride compound (b′) in the presence of a basic compound (d′), wherein the sulfonic acid fluoride compound (b′) is represented by the above formula (b1-1), the silylation agent can convert a hydroxy group on the nitrogen atom of the N-hydroxy compound (a′) into a silyloxy group represented by the above formula (ac1).

The N-hydroxy compound (a′) is a compound represented by the following formula (a3-2).

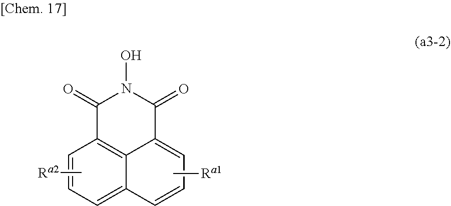

R^{a1} and R^{a2} in the formula (a3-2) are the same as R^{a1} and R^{a2} in the above formula (a3).

The N-hydroxy compound (a′) can be synthesized by routine procedure disclosed in, for example, Pamphlet of PCT International Publication No. WO2014/084269 and Japanese Unexamined Patent Application (Translation of PCT Application), Publication No. 2017-535595. For example, a compound represented by the formula (a3-2) in which R^{a1} is a hydrogen atom can be synthesized by converting the bromo group on the naphthalic anhydride to R^{a1} by a reaction represented by the following formula using a commercially available bromide as a starting material, and then acting a hydroxylamine compound such as hydroxylamine hydrochloride to the acid anhydride group to perform N-hydroxyimidization. As the N-hydroxy compound (a′), commercially available products may be used.

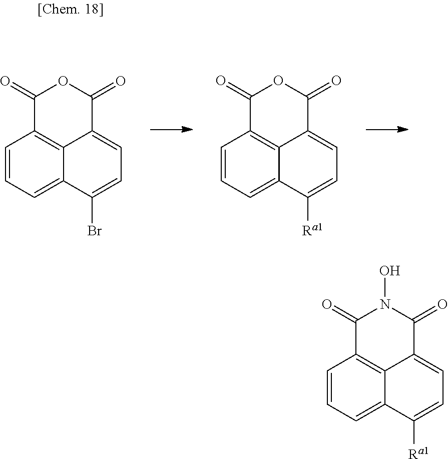

The sulfonic acid fluoride compound (b′) can be synthesized by routine procedure. For example, in (b1-1), compounds in which Q¹ and Q² are a fluorine atom can be synthesized by the reaction represented by the following formula. Furthermore, as the sulfonic acid fluoride compound (b′), commercially available products may be used.

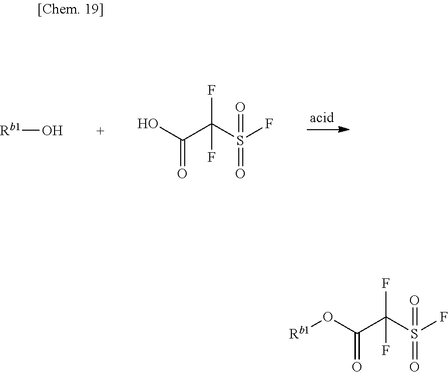

In the formula (ac1), the hydrocarbon group having 1 or more and 10 or less carbon atoms as R^{c1} may be an aliphatic hydrocarbon group, or an aromatic hydrocarbon group, or combination of these groups. The aliphatic hydrocarbon group may be linear or branched or cyclic, or combination of these structures. Examples of the aliphatic hydrocarbon group include alkyl groups such as a methyl group, an ethyl group, an n-propyl group, an isopropyl group, an n-butyl group, an isobutyl group, a sec-butyl group, a tert-butyl group, an n-pentyl group, an n-hexyl group, an n-heptyl group, an n-octyl group, a 2-ethyl hexyl group, an n-nonyl group, and an n-decyl group. Examples of the aromatic hydrocarbon group include a phenyl group and a naphthyl group.

Examples of the silylating agent (c′) include a compound represented by the formula (c′1).

X—Si(R^{c1})₃ (c′1)

(in the formula (c′1), R^{c1} is the same as R^{c1} in the formula (ac1), and X is a halogen atom).

Specific examples of the halogen atom as X in the formula (c′1) include a chlorine atom, a fluorine atom, a bromine atom, and an iodine atom.

Specific examples of the silylating agent (c′) include trimethylsilyl chloride, trimethylsilyl fluoride, trimethylsilyl bromide, t-butyldimethylsilyl chloride, ethyl dimethylsilyl chloride, isopropyl dimethylsilyl chloride.

The basic compound (d′) may be an organic base or an inorganic base. Examples of the organic base include a nitrogen-containing basic compound, for example, and specific examples thereof include amines such as methylamine, ethylamine, n-propylamine, isopropylamine, n-butylamine, dimethylamine, diethylamine, di-n-propylamine, diisopropylamine, di-n-butylamine, trimethylamine, triethylamine, methyl diethylamine, N-ethyldiisopropylamine, tri-n-propylamine, triisopropylamine, monoethanolamine, diethanolamine, and triethanolamine; a cyclic basic compound such as pyrrole, piperidine, 1,8-diazabicyclo[5,4,0]-7-undecene, and 1,5-diazabicyclo[4,3,0]-5-nonane; quaternary ammonium salts such as tetramethylammonium hydroxide (TMAH), tetraethylammonium hydroxide, tetrapropylammonium hydroxide (TPAH), tetrabutylammonium hydroxide, methyltripropylammonium hydroxide, methyltributylammonium hydroxide, benzyltrimethylammonium hydroxide, benzyltriethylammonium hydroxide, and trimethyl (2-hydroxyethyl) ammonium hydroxide, and the like. Examples of the inorganic base include, for example, metal hydroxides, metal hydrogen carbonate, and metal bicarbonates. Specific examples of inorganic bases include metal hydroxides such as lithium hydroxide, potassium hydroxide, sodium hydroxide, rubidium hydroxide, cesium hydroxide, magnesium hydroxide, calcium hydroxide, strontium hydroxide, and barium hydroxide; metal carbonates such as lithium carbonate, potassium carbonate, sodium carbonate, rubidium carbonate, cesium carbonate, magnesium carbonate, calcium carbonate, strontium carbonate, and barium carbonate; and metal bicarbonates such as lithium hydrogen carbonate, potassium hydrogen carbonate, sodium hydrogen carbonate, rubidium hydrogen carbonate, and cesium hydrogen carbonate.

In the method for producing an N-organosulfonyloxy compound, such N-hydroxy compound (a′) and sulfonic acid fluoride compound (b′) are reacted with each other in the presence of the silylating agent (c′) and the basic compound (d′). When the N-hydroxy compound (a′) and the sulfonic acid fluoride compound (b′) are reacted with each other in the presence of the basic compound (d′) in this way, the presence of the silylating agent (c′) can allow an N-organosulfonyloxy compound to be produced efficiently. For example, the N-organosulfonyloxy compound can be obtained in an amount of 65% or more with respect to the N-hydroxy compound (a′) and the sulfonic acid fluoride compound (b′) as raw materials.

The method for producing the N-organosulfonyloxy compound provides the N-organosulfonyloxy compound having a structure in which a group in which the hydrogen atom of the hydroxy group bonded to the nitrogen atom of the N-hydroxy compound (a′) is removed and R^{b1}—SO₂— derived from the sulfonic acid fluoride compound (b′) are bonded.

In the method for producing an N-organosulfonyloxy compound, when the N-hydroxy compound (a′) and the sulfonic acid fluoride compound (b′) are reacted with each other in the presence of a basic compound (d′), a silylating agent (c′) is only required to be present in a system, the N-hydroxy compound (a′), the sulfonic acid fluoride compound (b′), the silylating agent (c′), and the basic compound (d′) may be mixed simultaneously, after a part of the N-hydroxy compound (a′) and the silylating agent (c′) is reacted or after the reaction of the N-hydroxy compound (a′) and the silylating agent (c′) is completed, sulfonic acid fluoride (b′) and the basic compound (d′) may be added.

When such an N-hydroxy compound (a′) and a sulfonic acid fluoride compound (b′) are reacted with each other in the presence of the silylating agent (c′) and the basic compound (d′), the N-hydroxy compound (a′) is silylated with a silylating agent (c′), a hydroxyl group on the nitrogen atom is converted into a silyloxy group represented by the above formula (ac1) (Step 1: silylation step). Then, the silylated product of the N-hydroxy compound (a′) produced in the silylation step is fused with the sulfonic acid fluoride compound (b′) on which the basic compound (d′) acts (Step 2: condensation step). Thus, an N-organosulfonyloxy compound is obtained.

As one example of the method for producing an N-organosulfonyloxy compound, a reaction formula of a case where the compound represented by the above formula (a3-2) is used as the N-hydroxy compound (a′), a compound represented by the above formula (b1-1) in which Q¹ and Q² are a fluorine atom is used as the sulfonic acid fluoride compound (b′), trimethylsilyl chloride is used as the silylating agent (c′), and triethylamine is used as the basic compound (d′) is described below. Note here that the following is not a reaction mechanism determined analytically, but a reaction mechanism assumed from the raw materials and the behavior during the reaction.

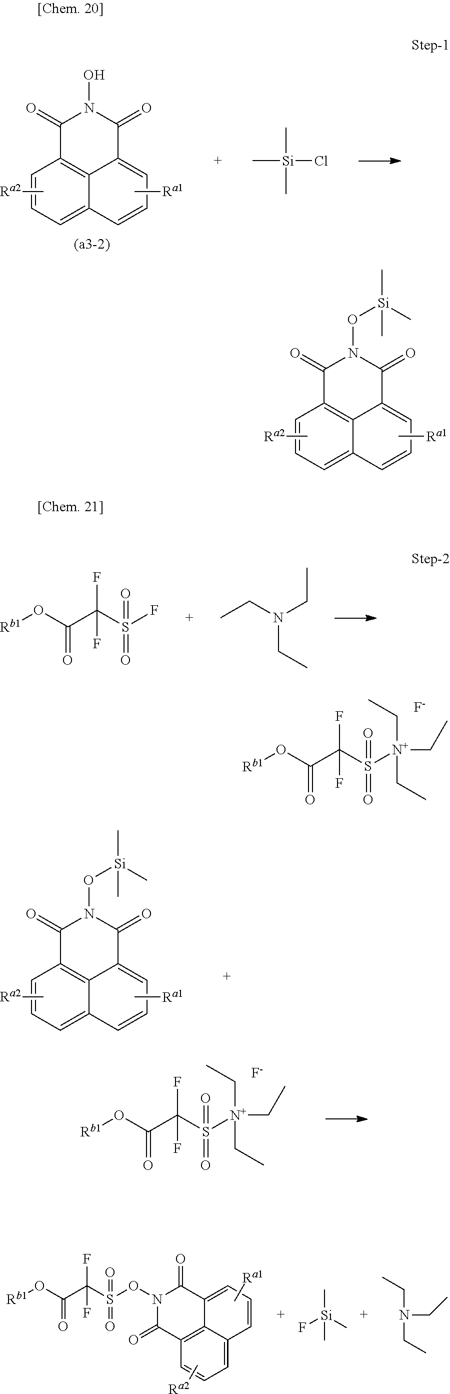

Examples of the organic solvent to be used for the reaction include esters such as ethyl acetate, butyl acetate, and cellosolve acetate; ketones such as acetone, methyl ethyl ketone, isobutyl ketone, and methyl isobutyl ketone; esters such as ethyl acetate, butyl acetate, and diethyl malonate; amides such as N-methylpyrrolidone and N, N-dimethylformamide; ethers such as diethyl ether, ethylcyclopentyl ether, tetrahydrofuran, and dioxane; aromatic hydrocarbons such as toluene and xylene; aliphatic hydrocarbons such as hexane, heptane, octane, and decahydronaphthalene; halogenated hydrocarbons such as chloroform, dichloromethane, methylene chloride, and ethylene chloride; nitrile solvents such as acetonitrile and propionitrile; dimethyl sulfoxide, dimethyl sulfoamide, and the like. One kind of organic solvent may be used, or two or more kinds thereof may be arbitrarily used in combination. A reaction temperature that can be employed is, for example, in a range of −10° C. to 200° C., preferably in a range of 0° C. to 150° C., and more preferably in a range of 5° C. to 120° C. A reaction time that can be employed is, for example, 5 minutes or more and 20 hours or less, 10 minutes or more, and 15 hours or less, and 30 minutes or more and 12 hours or less.

It is preferable that a sulfonic acid fluoride compound (b′), a silylating agent (c′), and a basic compound (d′) are respectively used in an excessive amount, with respect to the N-hydroxy compound (a′). For example, it is preferable that 1.1 mol or more and 2.5 mol or less of sulfonic acid fluoride compound (b′), 1.1 mol or more and 2.5 mol or less of silylating agent (c′), and 1.1 mol or more and 2.5 mol or less of basic compound (d′) are used with respect to 1.0 mol of N-hydroxy compound (a′).

The acid generating agent (A) may include an acid generating agent other than the non-ionic acid generating agent that generates sulfonic acid upon irradiation with active rays or radiation. A percentage of the non-ionic acid generating agent that generates sulfonic acid upon irradiation with active rays or radiation with respect to the mass of the acid generating agent (A) is preferably 50% by mass or more, more preferably 70% by mass or more, further preferably 80% by mass or more, further more preferably 90% by mass or more, particularly preferably 95% by mass or more, and the most preferably 100% by mass.

The total content of the acid generating agent (A) is preferably 0.01% by mass or more and 20% by mass or less, more preferably 0.03% by mass or more and 10% by mass or less, and particularly preferably 0.05% by mass or more and 8% by mass or less with respect to the total solid content of the photosensitive resin composition. Furthermore, the non-ionic acid generating agent that generates sulfonic acid upon irradiation with active rays or radiation is preferably 0.01% by mass or more and 20% by mass or less, preferably 0.03% by mass or more and 10% by mass or less, and particularly preferably 0.05% by mass or more and 8% by mass or less with respect to the total solid content of the photosensitive resin composition.

### <Resin (B)>

A resin (B) having alkali solubility that increases under action of acid is not particularly limited, and any resins in which solubility in alkali increases under the action of acid can be used. Among them, it is preferable to contain at least one resin selected from the group consisting of a novolac resin (B1), a polyhydroxystyrene resin (B2), and an acrylic resin (B3).

### [Novolac Resin (B1)]

As the novolak resin (B1), a resin including the constituent unit represented by the following formula (b1-11) may be used.

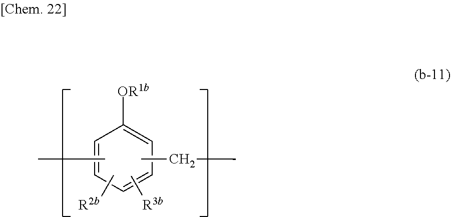

In the formula (b1-11), Rib represents an acid-dissociable dissolution-inhibiting group, and R^{2b} and R^{3b} each independently represents a hydrogen atom or an alkyl group having 1 or more and 6 or less carbon atoms.

The acid-dissociable dissolution-inhibiting group represented by the above R^{1b} is preferably a group represented by the following formula (b-12) or (b-13), a linear, branched, or cyclic alkyl group having 1 or more and 6 or less carbon atoms, a vinyloxyethyl group, a tetrahydropyranyl group, a tetrahydrofuranyl group, or a trialkylsilyl group.

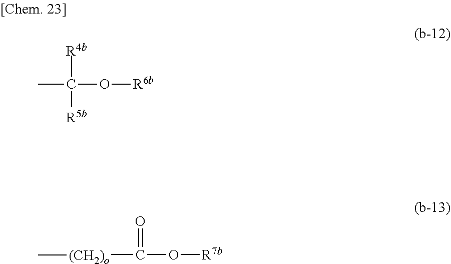

In the above formulae (b-12) and (b-13), Rob and R^{5b} each independently represents a hydrogen atom, or a linear or branched alkyl group having 1 or more and 6 or less carbon atoms, R^{6b} represents a linear, branched, or cyclic alkyl group having 1 or more and 10 or less carbon atoms, R^{7b} represents a linear, branched or cyclic alkyl group having 1 or more and 6 or less carbon atoms, and ∘ represents 0 or 1.

Examples of the above linear or branched alkyl group include a methyl group, an ethyl group, a propyl group, an isopropyl group, an n-butyl group, an isobutyl group, a tert-butyl group, a pentyl group, an isopentyl group, a neopentyl group, and the like. Examples of the above cyclic alkyl group include a cyclopentyl group, a cyclohexyl group, and the like.

Specific examples of the acid-dissociable dissolution-inhibiting group represented by the above formula (b-12) include a methoxyethyl group, ethoxyethyl group, n-propoxyethyl group, isopropoxyethyl group, n-butoxyethyl group, isobutoxyethyl group, tert-butoxyethyl group, cyclohexyloxyethyl group, methoxypropyl group, ethoxypropyl group, 1-methoxy-1-methyl-ethyl group, 1-ethoxy-1-methylethyl group, and the like. Furthermore, specific examples of the acid-dissociable dissolution-inhibiting group represented by the above formula (b-13) include a tert-butoxycarbonyl group, a tert-butoxycarbonylmethyl group, and the like. Examples of the above trialkylsilyl group include a trimethylsilyl group and tri-tert-butyldimethylsilyl group in which each alkyl group has 1 or more and 6 or less carbon atoms.

### [Polyhydroxystyrene Resin (B2)]

As the polyhydroxystyrene resin (B2), a resin including a constituent unit represented by the following formula (b4) may be used.

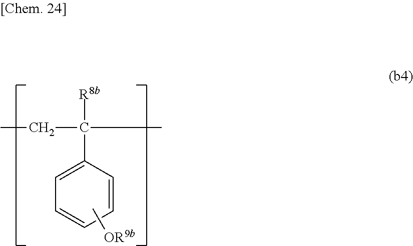

In the above formula (b4), R^{8b} represents a hydrogen atom or an alkyl group having 1 or more and 6 or less carbon atoms, and R^{9b} represents an acid-dissociable dissolution-inhibiting group.

The above alkyl group having 1 or more and 6 or less carbon atoms may include, for example, linear, branched, or cyclic alkyl groups having 1 or more and 6 or less carbon atoms. Examples of the linear or branched alkyl group include a methyl group, an ethyl group, a propyl group, an isopropyl group, an n-butyl group, an isobutyl group, a tert-butyl group, a pentyl group, an isopentyl group, a neopentyl group, and the like. Examples of the cyclic alkyl group include a cyclopentyl group and a cyclohexyl group.

Examples of the acid-dissociable dissolution-inhibiting group represented by the above R^{9b} include the groups the same as the acid-dissociable dissolution-inhibiting group exemplified in terms of the above formulae (b-12) and (b-13).

Furthermore, the polyhydroxystyrene resin (B2) may include another polymerizable compound as a constituent unit in order to moderately control physical or chemical properties. The polymerizable compound is exemplified by conventional radical polymerizable compounds and anion polymerizable compounds. Examples of the polymerizable compound include, for example, monocarboxylic acids such as acrylic acid, methacrylic acid and crotonic acid; dicarboxylic acids such as maleic acid, fumaric acid and itaconic acid; methacrylic acid derivatives having a carboxyl group and an ester bond such as 2-methacryloyloxyethyl succinic acid, 2-methacryloyloxyethyl maleic acid, 2-methacryloyloxyethyl phthalic acid and 2-methacryloyloxyethyl hexahydrophthalic acid; (meth)acrylic acid alkyl esters such as methyl(meth)acrylate, ethyl (meth)acrylate and butyl (meth)acrylate; (meth)acrylic acid hydroxyalkyl esters such as 2-hydroxyethyl (meth)acrylate and 2-hydroxypropyl (meth)acrylate; (meth)acrylic acid aryl esters such as phenyl (meth)acrylate and benzyl (meth)acrylate; dicarboxylic acid diesters such as diethyl maleate and dibutyl fumarate; vinyl group-containing aromatic compounds such as styrene, α-methylstyrene, chlorostyrene, chloromethylstyrene, vinyltoluene, hydroxystyrene, α-methylhydroxystyrene and α-ethylhydroxystyrene; vinyl group-containing aliphatic compounds such as vinyl acetate; conjugated diolefins such as butadiene and isoprene; nitrile group-containing polymerizable compounds such as acrylonitrile and methacrylonitrile; chlorine-containing polymerizable compounds such as vinyl chloride and vinylidene chloride; and amide bond-containing polymerizable compounds such as acrylamide and methacrylamide.

### [Acrylic Resin (B3)]

An acrylic resin (B3) is not particularly limited as long as it is an acrylic resin the solubility of which in alkali increases under the action of acid, and has conventionally blended in various photosensitive resin compositions. Preferably, the acrylic resin (B3) contains a constituent unit (b-3) derived from, for example, an acrylic ester including an —SO₂-containing cyclic group or a lactone-containing cyclic group. In such a case, when a resist pattern is formed, a resist pattern having a preferable cross-sectional shape can be easily formed.

### (—SO₂-Containing Cyclic Group)

Herein, the “—SO₂-containing cyclic group” refers to a cyclic group having a cyclic group containing a ring including —SO₂— in the ring skeleton thereof, and specifically is a cyclic group in which the sulfur atom (S) in —SO₂— forms a part of the ring skeleton of the cyclic group. Considering a ring including —SO₂— in the ring skeleton thereof as the first ring, a group having the ring alone is called a monocyclic group, and a group further having another ring structure is called a polycyclic group regardless of its structure. The —SO₂— containing cyclic group may be monocyclic or polycyclic.

In particular, the —SO₂-containing cyclic group is preferably a cyclic group containing —O—SO₂— in the ring skeleton thereof, i.e., a cyclic group containing a sultone ring in which —O—S— in —O—SO₂— forms a part of the ring skeleton.

The number of carbon atoms in an —SO₂-containing cyclic group is preferably 3 or more and 30 or less, more preferably 4 or more and 20 or less, even more preferably 4 or more and 15 or less, and in particular preferably 4 or more and 12 or less. The above number of carbon atoms is the number of carbon atoms constituting a ring skeleton, and shall not include the number of carbon atoms in a substituent.

The —SO₂-containing cyclic group may be an —SO₂— containing aliphatic cyclic group or an —SO₂-containing aromatic cyclic group. It is preferably an —SO₂-containing aliphatic cyclic group.

—SO₂— containing aliphatic cyclic groups include a group in which at least one hydrogen atom is removed from an aliphatic hydrocarbon ring where a part of the carbon atoms constituting the ring skeleton thereof is(are) substituted with —SO₂— or —O—SO₂—. More specifically, examples include a group in which at least one hydrogen atom is removed from an aliphatic hydrocarbon ring where —CH₂— constituting the ring skeleton thereof is substituted with —SO₂— and a group in which at least one hydrogen atom is removed from an aliphatic hydrocarbon ring where —CH₂—CH₂— constituting the ring thereof is substituted with —O—SO₂—.

The number of carbon atoms in the above alicyclic hydrocarbon ring is preferably 3 or more and 20 or less, and more preferably 3 or more and 12 or less. The above alicyclic hydrocarbon ring may be polycyclic, or may be monocyclic. As the monocyclic alicyclic hydrocarbon group, preferred is a group in which two hydrogen atoms are removed from monocycloalkane having 3 or more and 6 or less carbon atoms. Examples of the above monocycloalkane can include cyclopentane, cyclohexane and the like. As the polycyclic alicyclic hydrocarbon ring, preferred is a group in which two hydrogen atoms are removed from polycycloalkane having 7 or more and 12 or less carbon atoms, and specific examples of the above polycycloalkane include adamantane, norbornane, isobornane, tricyclodecane, tetracyclododecane and the like.

The —SO₂-containing cyclic group may have a substituent. Examples of the substituent include, for example, an alkyl group, an alkoxy group, a halogen atom, a halogenated alkyl group, a hydroxyl group, an oxygen atom (═O), —COOR″, —OC(═O)R″, a hydroxyalkyl group, a cyano group and the like.

For an alkyl group as the substituent, an alkyl group having 1 or more and 6 or less carbon atom is preferable. The alkyl group is preferably linear or branched. Specific examples include a methyl group, an ethyl group, an n-propyl group, an isopropyl group, an n-butyl group, an isobutyl group, a tert-butyl group, an n-pentyl group, an isopentyl group, a neopentyl group, an n-hexyl group and the like. Among these, a methyl group or an ethyl group is preferable, and a methyl group is particularly preferable.

For an alkoxy group as the substituent, an alkoxy group having 1 or more and 6 or less carbon atoms is preferable. The above alkoxy group is preferably linear or branched. Specific examples include a group in which an alkyl group recited as an alkyl group for the above substituent is attached to the oxygen atom (—O—).

Halogen atoms as the substituent include a fluorine atom, a chlorine atom, a bromine atom, an iodine atom and the like, and a fluorine atom is preferable.

Halogenated alkyl groups for the substituent include a group in which a part or all of the hydrogen atoms in the above alkyl group is(are) substituted with the above halogen atom(s).

Halogenated alkyl groups as the substituent include a group in which a part or all of the hydrogen atoms in the alkyl groups recited as an alkyl group for the above substituent is(are) substituted with the above halogen atom(s). As the above halogenated alkyl group, a fluorinated alkyl group is preferable, and a perfluoroalkyl group is particularly preferable.

R″s in the aforementioned —COOR″ and —OC(═O)R″ are either a hydrogen atom or a linear, branched, or cyclic alkyl group having 1 or more and 15 or less carbon atoms.

In a case where R″ is a linear or branched alkyl group, the number of carbon atoms in the chain alkyl group is preferably 1 or more and 10 or less, more preferably 1 or more and 5 or less, and particularly preferably 1 or 2.

In a case where R″ is a cyclic alkyl group, the number of carbon atoms in the above cyclic alkyl group is preferably 3 or more and 15 or less, more preferably 4 or more and 12 or less, and in particular preferably 5 or more and 10 or less. Specific examples can include a group in which one or more hydrogen atoms are removed from monocycloalkane, and polycycloalkane such as bicycloalkane, tricycloalkane, tetracycloalkane and the like optionally substituted with a fluorine atom or a fluorinated alkyl group. More specific examples include a group in which one or more hydrogen atoms are removed from monocycloalkane such as cyclopentane and cyclohexane, and polycycloalkane such as adamantane, norbornane, isobornane, tricyclodecane and tetracyclododecane.

For a hydroxyalkyl group as the substituent, a hydroxyalkyl group having 1 or more and 6 or less carbon atoms is preferable. Specific examples include a group in which at least one of the hydrogen atoms in the alkyl groups recited as an alkyl group for the above substituent is substituted with a hydroxyl group.

More specific examples of the —SO₂-containing cyclic group include the groups represented by the following formulae (3-1) to (3-4).

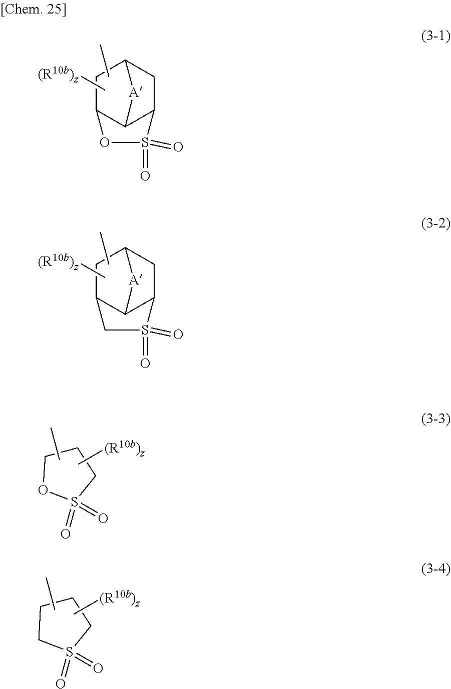

(In the formula, A′ represents an alkylene group having 1 or more and 5 or less carbon atoms optionally including an oxygen atom or a sulfur atom, an oxygen atom or a sulfur atom; z represents an integer of 0 or more and 2 or less; R^{10b} represents an alkyl group, an alkoxy group, a halogenated alkyl group, a hydroxyl group, —COOR″, —OC(═O)R″, a hydroxyalkyl group, or a cyano group; and R″ represents a hydrogen atom or an alkyl group).

In the above formulae (3-1) to (3-4), A′ represents an alkylene group having 1 or more and 5 or less carbon atoms optionally including an oxygen atom (—O—) or a sulfur atom (—S—), an oxygen atom or a sulfur atom. As an alkylene group having 1 or more and 5 or less carbon atoms in A′, a linear or branched alkylene group is preferred, and examples thereof include a methylene group, an ethylene group, an n-propylene group, an isopropylene group and the like.

In a case where the above alkylene group includes an oxygen atom or a sulfur atom, specific examples thereof include a group in which —O— or —S— is present at a terminal or between carbon atoms of the above alkylene group, for example, —O—CH₂—, —CH₂—O—CH₂—, —S—CH₂—, —CH₂—S—CH₂—, and the like. As A′, an alkylene group having 1 or more and 5 or less carbon atoms or —O— is preferred, and an alkylene group having 1 or more and 5 or less carbon atoms is more preferred, and a methylene group is most preferred.

z may be any of 0, 1, and 2, and is most preferably 0. In a case where z is 2, a plurality of R^{10b}s may be the same, or may differ from each other.

An alkyl group, a alkoxy group, a halogenated alkyl group, —COOR″, —OC(═O)R″ and a hydroxyalkyl group in R^{10b} include the same as described above for the alkyl group, the alkoxy group, the halogenated alkyl group, —COOR″, —OC(═O)R″ and the hydroxyalkyl group, respectively, which are recited as those optionally contained in the —SO₂-containing cyclic group.

Hereinafter, specific cyclic groups represented by the above formulae (3-1) to (3-4) will be illustrated. Note here that “Ac” in the formulae represents an acetyl group.

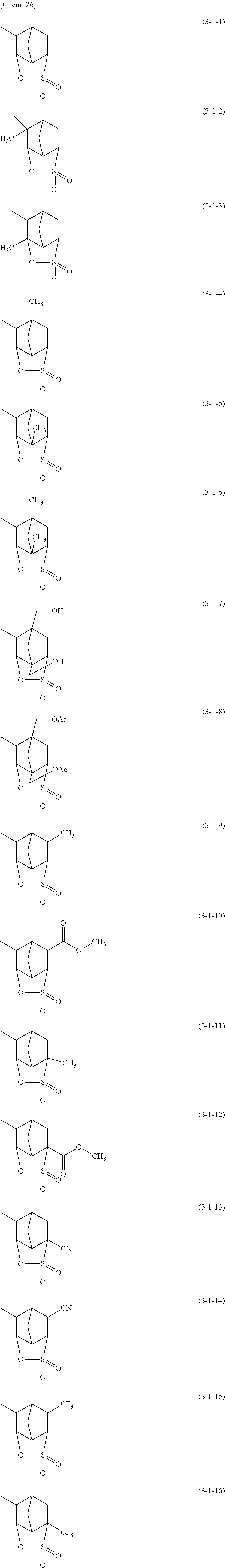

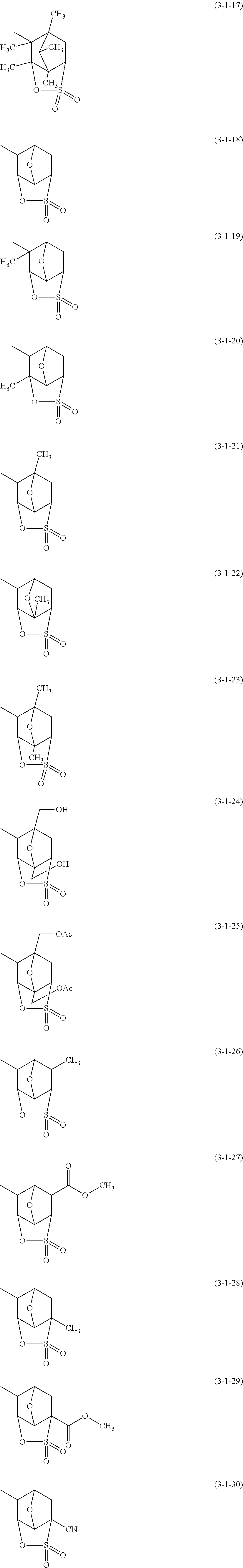

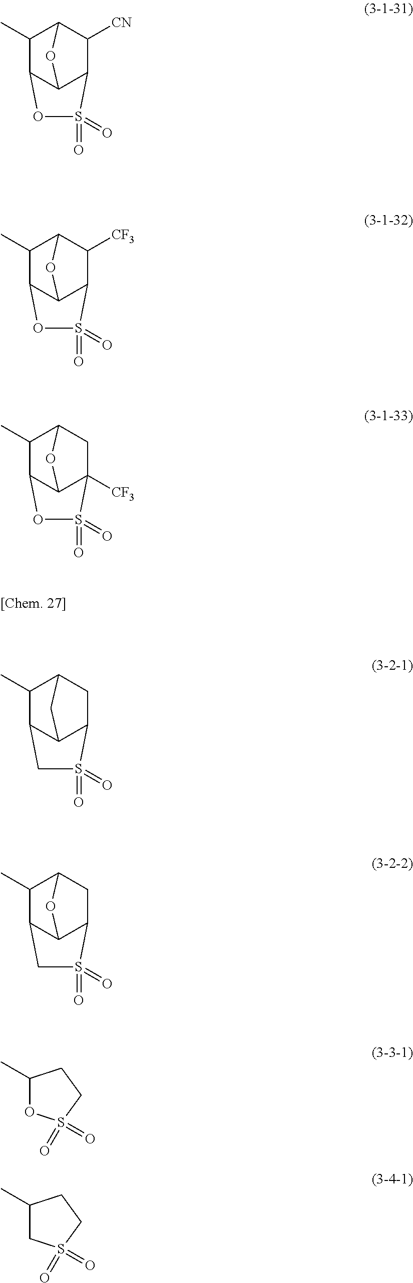

As the —SO₂-containing cyclic group, among those shown above, a group represented by the above formula (3-1) is preferred, and at least one group selected from the group consisting of the groups represented by any of the aforementioned chemical formulae (3-1-1), (3-1-18), (3-3-1) and (3-4-1) is more preferred, and a group represented by the aforementioned chemical formula (3-1-1) is most preferred.

### (Lactone-Containing Cyclic Group)

The “lactone-containing cyclic group” refers to a cyclic group containing a ring (lactone ring) including —O—C(═O)— in the ring skeleton thereof. Considering the lactone ring as the first ring, a group having that lactone ring alone is called a monocyclic group, and a group further having another ring structure is called a polycyclic group regardless of its structure. The lactone-containing cyclic group may be a monocyclic group, or may be a polycyclic group.

There is no particular limitation on the lactone cyclic group in the constituent unit (b-3), and any cyclic group can be used. Specifically, examples of the lactone-containing monocyclic groups include a group in which one hydrogen atom is removed from 4 to 6 membered ring lactone, for example, a group in which one hydrogen atom is removed from β-propionolactone, a group in which one hydrogen atom is removed from γ-butyrolactone, a group in which one hydrogen atom is removed from δ-valerolactone and the like. Further, lactone-containing polycyclic groups include a group in which one hydrogen atom is removed from bicycloalkane, tricycloalkane and tetracycloalkane having a lactone ring.

As the constituent unit (b-3), as long as the constituent unit (b-3) has an —SO₂-containing cyclic group or a lactone-containing cyclic group, the structures of other parts are not particularly limited. A preferable constituent unit (b-3) is at least one constituent unit selected from the group consisting of a constituent unit (b-3-S) derived from an acrylic acid ester including an —SO₂-containing cyclic group in which a hydrogen atom bonded to the carbon atom in the a position may be substituted with a substituent; and a constituent unit (b-3-L) derived from an acrylic acid ester including a lactone-containing cyclic group in which the hydrogen atom bonded to the carbon atom in the a position may be substituted with a substituent.

[Constituent Unit (b-3-S)]

More specific examples of the constituent unit (b-3-S) include one represented by the following formula (b-S1).

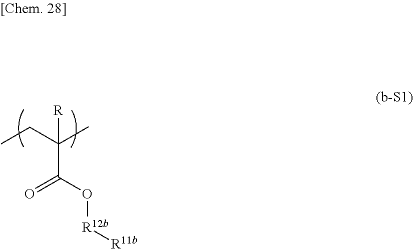

(In the formula, R represents a hydrogen atom, an alkyl group having 1 or more 5 or less carbon atoms or a halogenated alkyl group having 1 or more 5 or less carbon atoms; and R^{11b} represents an —SO₂-containing cyclic group; and R^{12b} represents a single-bond or divalent linking group.)

In the formula (b-S1), R is the same as the above. R^{11b} is the same as in the —SO₂-containing cyclic group described above. R^{12b} may be either a single-bond linking group or a divalent linking group.

The divalent linking group in R^{12b} is not particularly limited, but suitable groups include an optionally substituted divalent hydrocarbon group, a divalent linking group including a heteroatom, and the like.

Optionally Substituted Divalent Hydrocarbon Group

The hydrocarbon group as a divalent linking group may be an aliphatic hydrocarbon group, or may be an aromatic hydrocarbon group. The aliphatic hydrocarbon group means a hydrocarbon group without having aromaticity. The above aliphatic hydrocarbon group may be saturated or may be unsaturated. Usually, a saturated hydrocarbon group is preferable. More specific examples of the above aliphatic hydrocarbon group include a linear or branched aliphatic hydrocarbon group, an aliphatic hydrocarbon group including a ring in the structure thereof and the like.

The number of carbon atoms in the linear or branched aliphatic hydrocarbon group is preferably 1 or more and 10 or less, more preferably 1 or more and 8 or less, and further preferably 1 or more and 5 or less.

As the linear aliphatic hydrocarbon group, a linear alkylene group is preferable. Specific examples include a methylene group [—CH₂—], an ethylene group [—(CH₂)₂—], a trimethylene group [—(CH₂)₃—], a tetramethylene group [—(CH₂)₄—], a pentamethylene group [—(CH₂)₅-] and the like.

As the branched aliphatic hydrocarbon group, a branched alkylene group is preferable. Specific examples include alkyl alkylene groups such as alkyl methylene groups such as —CH(CH₃)—, —CH(CH₂CH₃)—, —C(CH₃)₂—, —C(CH₃) (CH₂CH₃)—, —C(CH₃) (CH₂CH₂CH₃)—, and —C(CH₂CH₃)₂-; alkyl ethylene groups such as —CH(CH₃) CH₂—, —CH(CH₃) CH(CH₃)—, —C(CH₃)₂CH₂—, —CH(CH₂CH₃) CH₂—, and —C(CH₂CH₃)₂—CH₂—; alkyl trimethylene groups such as —CH(CH₃) CH₂CH₂— and —CH₂CH(CH₃) CH₂—; alkyl tetramethylene groups such as —CH(CH₃)CH₂CH₂CH₂— and —CH₂CH(CH₃)CH₂CH₂—; and the like. As an alkyl group in the alkyl alkylene group, a linear alkyl group having 1 or more and 5 or less carbon atoms is preferable.

The above linear or branched aliphatic hydrocarbon group may or may not have a substituent (a group or atom other than a hydrogen atom) which substitutes a hydrogen atom. Examples of the substituent include a fluorine atom, a fluorinated alkyl group having 1 or more and 5 or less carbon atoms substituted with a fluorine atom, an oxo group (═O) and the like.

Examples of the above aliphatic hydrocarbon group including a ring in the structure thereof include a cyclic aliphatic hydrocarbon group optionally including a hetero atom in the ring structure (a group in which two hydrogen atoms are removed from an aliphatic hydrocarbon ring); a group in which the above cyclic aliphatic hydrocarbon group is bonded to an end of a linear or branched aliphatic hydrocarbon group; a group in which the above cyclic aliphatic hydrocarbon group is present in the middle of the linear or branched aliphatic hydrocarbon group; and the like. Examples of the above linear or branched aliphatic hydrocarbon group include those the same as the above.

The number of carbon atoms in the cyclic aliphatic hydrocarbon group is preferably 3 or more and 20 or less, and more preferably 3 or more and 12 or less.

The cyclic aliphatic hydrocarbon group may be polycyclic, or may be monocyclic. As the monocyclic aliphatic hydrocarbon group, a group in which two hydrogen atoms are removed from monocycloalkane is preferable. The number of carbon atoms in the monocycloalkane is preferably 3 or more and 6 or less. Specific examples include cyclopentane, cyclohexane and the like. As the polycyclic aliphatic hydrocarbon group, a group in which two hydrogen atoms are removed from polycycloalkane is preferable. The number of carbon atoms in the polycycloalkane is preferably 7 or more and 12 or less. Specific examples include adamantane, norbornane, isobornane, tricyclodecane, tetracyclododecane and the like.

The cyclic aliphatic hydrocarbon group may or may not have a substituent which substitutes a hydrogen atom (a group or atom other than a hydrogen atom). Examples of the substituent include an alkyl group, an alkoxy group, a halogen atom, a halogenated alkyl group, a hydroxyl group, an oxo group (═O) and the like.

For an alkyl group as a substituent mentioned above, an alkyl group having 1 or more and 5 or less carbon atoms is preferable, and a methyl group, an ethyl group, a propyl group, an n-butyl group and a tert-butyl group are more preferable.

For an alkoxy group a substituent mentioned above, an alkoxy group having 1 or more and 5 or less carbon atoms is preferable, and a methoxy group, an ethoxy group, an n-propoxy group, an iso-propoxy group, an n-butoxy group and a tert-butoxy group are more preferable, and a methoxy group and an ethoxy group are particularly preferable.

Halogen atoms as a substituent mentioned above include a fluorine atom, a chlorine atom, a bromine atom, an iodine atom and the like, and a fluorine atom is preferable.

The halogenated alkyl groups as a substituent mentioned above include a group in which a part or all of hydrogen atoms in the aforementioned alkyl group is(are) substituted with the above halogen atom(s).

In the cyclic aliphatic hydrocarbon group, a part of carbon atoms constituting the ring structure thereof may be substituted with —O—, or —S—. As the substituent including the hetero atom, —O—, —C(═O)—O—, —S—, —S(═O)₂—, and —S(═O)₂—O— are preferable.

The aromatic hydrocarbon group as the divalent hydrocarbon group is a divalent hydrocarbon group having at least one aromatic ring, and may have a substituent. The aromatic ring is not particularly limited as long as it is a cyclic conjugated system having a 4n+2 n electrons, and it may be monocyclic or may be polycyclic. The number of carbon atoms in the aromatic ring is preferably 5 or more and 30 or less, more preferably 5 or more and 20 or less, further more preferably 6 or more and 15 or less, and particularly preferably 6 or more and 12 or less. However, the number of carbon atoms in a substituent shall not be included in the above number of carbon atoms.

Specific examples of the aromatic rings include aromatic hydrocarbon rings such as benzene, naphthalene, anthracene, and phenanthrene; aromatic heterocycles in which a part of the carbon atoms constituting the above aromatic hydrocarbon ring is(are) substituted with hetero atom(s). Hetero atoms in the aromatic heterocycle include an oxygen atom, a sulfur atom, a nitrogen atom and the like. Specific examples of the aromatic heterocycles include a pyridine ring, a thiophene ring, and the like.

Specific examples of the aromatic hydrocarbon group as a divalent hydrocarbon group include a group in which two hydrogen atoms are removed from the above aromatic hydrocarbon ring or the above aromatic heterocycle (an arylene group or a heteroarylene group); a group in which two hydrogen atoms are removed from an aromatic compound including two or more aromatic rings (for example, biphenyl, fluorene and the like); a group in which one hydrogen atom from a group where one hydrogen atom is removed from the above aromatic hydrocarbon ring or the above aromatic heterocycle (an aryl group or a heteroaryl group) is substituted with an alkylene group (for example, a group in which one hydrogen atom is further removed from an aryl group in an arylalkyl group such as a benzyl group, a phenethyl group, a 1-naphthylmethyl group, a 2-naphthylmethyl group, a 1-naphthylethyl group, and a 2-naphthylethyl group); and the like.

The number of carbon atoms in the above alkylene group bonded to an aryl group or a heteroaryl group is preferably 1 or more and 4 or less, more preferably 1 or more and 2 or less, and particularly preferably 1.

In the above aromatic hydrocarbon group, a hydrogen atom of the aromatic hydrocarbon group may be substituted with a substituent. For example, a hydrogen atom bonded to an aromatic ring in the aromatic hydrocarbon group may be substituted with a substituent. Examples of the substituent include an alkyl group, an alkoxy group, a halogen atom, a halogenated alkyl group, a hydroxyl group, an oxo group (═O) and the like.

For an alkyl group as the above substituent, an alkyl group having 1 or more and 5 or less carbon atoms is preferred, and a methyl group, an ethyl group, an n-propyl group, an n-butyl group, and a tert-butyl group are more preferred.

For an alkoxy group as the substituent mentioned above, an alkoxy group having 1 or more and 5 or less carbon atoms is preferable, and a methoxy group, an ethoxy group, an n-propoxy group, an iso-propoxy group, an n-butoxy group, and a tert-butoxy group are preferable, and a methoxy group and an ethoxy group are more preferable.

Halogen atoms as the substituent mentioned above include a fluorine atom, a chlorine atom, a bromine atom, an iodine atom and the like, and a fluorine atom is preferable.

The halogenated alkyl groups as the substituent mentioned above include a group in which a part or all of hydrogen atoms in the aforementioned alkyl group is(are) substituted with the above halogen atom(s).

Divalent Linking Group Including Hetero Atom

A hetero atom in the divalent linking group including a hetero atom is an atom other than a carbon atom and a hydrogen atom, and examples thereof include an oxygen atom, a nitrogen atom, a sulfur atom, a halogen atom and the like.

Specific examples of the divalent linking group including a hetero atom include non-hydrocarbon based linking groups such as —O—, —C(═O)—, —C(═O)—O—, —O—C(═O)—O—, —S—, —S(═O)₂—, —S(═O)₂—O—, —NH—, —NH—C(═O)—, —NH—C(═NH)—, ═N—, and combinations of at least one of these non-hydrocarbon based linking groups and a divalent hydrocarbon group and the like. Examples of the above divalent hydrocarbon group include those the same as the aforementioned divalent hydrocarbon groups optionally having a substituent, and linear or branched aliphatic hydrocarbon groups are preferred.

Among those described above, —NH— in —C(═O)—NH—, and H in —NH— and —NH—C(═NH)— may be substituted with a substituent such as an alkyl group or an acyl group, respectively. The number of carbon atoms in the substituent is preferably 1 or more and 10 or less, more preferably 1 or more and 8 or less, and in particular preferably 1 or more and 5 or less.

As a divalent linking group in R^{12b}, a linear or branched alkylene group, a cyclic aliphatic hydrocarbon group, or a divalent linking group including a hetero atom is particularly preferable.

In a case where the divalent linking group in R^{12b} is a linear or branched alkylene group, the number of carbon atoms in the alkylene group is preferably 1 or more and 10 or less, more preferably 1 or more and 6 or less, in particular preferably 1 or more and 4 or less, and most preferably 1 or more and 3 or less. Specific examples include groups the same as the linear alkylene groups or branched alkylene groups recited as a linear and branched aliphatic hydrocarbon group in the description of the “divalent hydrocarbon group optionally having a substituent” as the aforementioned divalent linking group.

In a case where the divalent linking group in R^{12b} is a cyclic aliphatic hydrocarbon group, examples of the cyclic aliphatic hydrocarbon group include groups the same as the cyclic aliphatic hydrocarbon group described as the “aliphatic hydrocarbon group including a ring in the structure” in the description of the “divalent hydrocarbon group optionally having a substituent” as the aforementioned divalent linking group.

Particularly preferable examples of the cyclic aliphatic hydrocarbon group include a group in which two or more hydrogen atoms are removed from cyclopentane, cyclohexane, norbornane, isobornane, adamantane, tricyclodecane, or tetracyclododecane.

In a case where the divalent linking group in R^{12b} is a divalent linking group including a hetero atom, groups preferred as the above linking groups include —O—, —C(═O)—O—, —C(═O)—, —O—C(═O)—O—, —C(═O)—NH—, —NH— (H may be substituted with a substituent such as an alkyl group or an acyl group), —S—, —S(═O)₂—, —S(═O)₂—O—, and a group represented by the general formula —Y^{1b}—O—Y^{2b}—, —[Y^{1b}—C(═O)—O]_{n′}—Y^{2b}— or —Y^{1b}—O—C(═O)—Y^{2b}— [wherein Y^{1b} and Y^{2b} are divalent hydrocarbon groups each independently, optionally having a substituent, and O represents an oxygen atom, and m′ is an integer of 0 or more and 3 or less].

In a case where the divalent linking group in R^{12b} is —NH—, the hydrogen atom in —NH— may be substituted with a substituent such as an alkyl group or an acyl group. The number of carbon atoms in the above substituent (an alkyl group, an acyl group and the like) is preferably 1 or more and 10 or less, more preferably 1 or more and 8 or less, and in particular preferably 1 or more and 5 or less.

Y^{1b} and Y^{2b} in the formula Y_{1b}—O—Y^{2b}—, —[Y^{1b}—C(═O)—O]_{n′}—Y^{2b}—, or —Y^{1b}—O—C(═O)—Y^{2b}— are divalent hydrocarbon groups each independently, optionally having a substituent. Examples of the above divalent hydrocarbon group include groups the same as the “divalent hydrocarbon group optionally having a substituent” recited in the description of the above divalent linking group.

As Y^{1b}, a linear aliphatic hydrocarbon group is preferred, and a linear alkylene group is more preferred, and a linear alkylene group having 1 or more and 5 or less carbon atoms is more preferred, and a methylene group and an ethylene group are particularly preferable.

As Y^{2b}, a linear or branched aliphatic hydrocarbon group is preferable, and a methylene group, an ethylene group, and an alkylmethylene group are more preferable. The alkyl group in the alkylmethylene group is preferably a linear alkyl group having 1 or more and 5 or less carbon atoms, more preferably a linear alkyl group having 1 or more and 3 or less carbon atoms, and particularly preferably a methyl group.

In a group represented by the formula —[Y^{1b}—C(═O)—O]_{n′}—Y^{2b}—, m′ is an integer of 0 or more and 3 or less, preferably an integer of 0 or more and 2 or less, more preferably 0 or 1, and particularly preferably 1. In other words, as a group represented by the formula —[Y^{1b}—C(═O)—O]_{n′}—Y^{2b}—, a group represented by the formula —Y^{1b}—C(═O)—O—Y^{2b}— is particularly preferred. Among these, a group represented by the formula —(CH₂)_{a′}—C(═O)—O—(CH₂)_{b′}— is preferable. In the above formula, a′ is an integer of 1 or more and 10 or less, preferably an integer of 1 or more and 8 or less, more preferably an integer of 1 or more and 5 or less, even more preferably 1 or 2, and most preferably 1. b′ is an integer of 1 or more and 10 or less, preferably an integer of 1 or more and 8 or less, more preferably an integer of 1 or more and 5 or less, even more preferably 1 or 2, and most preferably 1.

With regard to the divalent linking group in R^{12b}, an organic group including a combination of at least one non-hydrocarbon group and a divalent hydrocarbon group is preferable as the divalent linking group including a hetero atom. Among these, a linear chain group having an oxygen atom as a hetero atom, for example, a group including an ether bond or an ester bond is preferable, and a group represented by the aforementioned formula —Y^{1b}—O—Y^{2b}—, —[Y^{1b}—C(═O)—O]^{m′}—Y^{2b}— or —Y^{1b}—O—C(═O)—Y^{2b}— is more preferable, and a group represented by the aforementioned formula —[Y^{1b}—C(═O)—O]_{m′}—Y^{2b}— or —Y^{1b}—O—C(═O)—Y^{2b}— is particularly preferable.

The divalent linking group in R^{12b} preferably include a group including an alkylene group or an ester bond (—C(═O)—O—).

The alkylene group is preferably a linear or branched alkylene group. Suitable examples of the linear aliphatic hydrocarbon group include a methylene group [—CH₂—], an ethylene group [—(CH₂)₂—], a trimethylene group [—(CH₂)₃—], a tetramethylene group [—(CH₂)₄—], a pentamethylene group [—(CH₂)₅—], and the like. Suitable examples of the branched alkylene group include alkyl alkylene groups such as alkyl methylene groups such as —CH(CH₃)—, —CH(CH₂CH₃)—, —C(CH₃)₂—, —C(CH₃) (CH₂CH₃)—, —C(CH₃) (CH₂CH₂CH₃)—, and —C(CH₂CH₃)₂—; alkyl ethylene groups such as —CH(CH₃) CH₂—, —CH(CH₃) CH(CH₃)—, —C(CH₃)₂CH₂—, —CH(CH₂CH₃) CH₂—, and —C(CH₂CH₃)₂—CH₂—; alkyl trimethylene groups such as —CH(CH₃)CH₂CH₂— and —CH₂CH(CH₃)CH₂—; alkyl tetramethylene groups such as —CH(CH₃)CH₂CH₂CH₂— and —CH₂CH(CH₃) CH₂CH₂—.

As the divalent linking group including an ester bond, in particular, a group represented by the formula: —R^{13b}—C(═O)—O—[in the formula, R^{13b} represents a divalent linking group] is preferable. In other words, the constituent unit (b-3-S) is preferably a constituent unit represented by the following formula (b-S1-1).

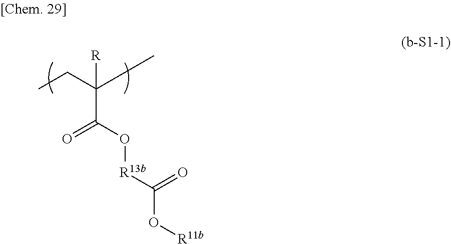

(In the formula, R and R^{11b} are the same as the above, and R^{13b} represents a divalent linking group.)

R^{13b} is not particularly limited, and examples thereof include groups the same as the divalent linking group described for R^{12b}. As the divalent linking group in R^{13b}, a linear or branched alkylene group, an aliphatic hydrocarbon group including a ring in the structure, or a divalent linking group including a hetero atom is preferable, and a linear or branched alkylene group or a divalent linking group including an oxygen atom as a hetero atom is preferable.

As the linear alkylene group, a methylene group or an ethylene group is preferable, and a methylene group is particularly preferable. As the branched alkylene group, an alkylmethylene group or an alkylethylene group is preferable, and —CH(CH₃)—, —C(CH₃)₂—, or —C(CH₃)₂CH₂— is particularly preferable.

As the divalent linking group including an oxygen atom, a divalent linking group including an ether bond or an ester bond is preferred, and the aforementioned —Y^{1b}—O—Y^{2b}—, —[Y^{1b}—C(═O)—O]_{m′}—Y^{2b}—, or —Y^{1b}—O—C(═O)—Y^{2b}— is more preferable. Y^{1b} and Y^{2b} are each independently divalent hydrocarbon groups optionally having a substituent, and m′ is an integer of 0 or more and 3 or less. Among these, —Y^{1b}—O—C(═O)—Y^{2b}— is preferred, and a group represented by —(CH₂)_{c}—O—C(═O)—(CH₂)_{d}— is particularly preferable. c is an integer of 1 or more and 5 or less, and 1 or 2 is preferable. d is an integer of 1 or more and 5 or less, and 1 or 2 is preferable.

As the constituent unit (b-3-S), in particular, one represented by the following formula (b-S1-11) or (b-S1-12) is preferable, and one represented by the formula (b-S1-12) is more preferable.

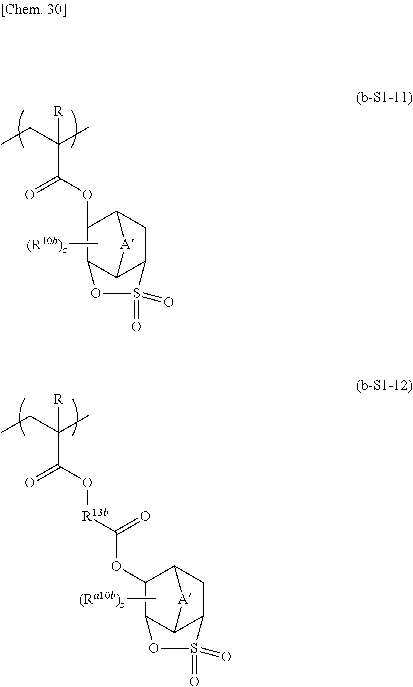

(in the formula, R, A′, R^{10b}, z, and R^{13b} are each the same as the above).

In the formula (b-S1-11), A′ is preferably a methylene group, an oxygen atom (—O—), or a sulfur atom (—S—).

R^{13b} is preferably a linear or branched alkylene group or a divalent linking group including an oxygen atom. Examples of the linear or branched alkylene group and the divalent linking group including an oxygen atom in R^{13b} include those the same as the aforementioned linear or branched alkylene group and the aforementioned divalent linking group including an oxygen atom, respectively.

As the constituent unit represented by the formula (b-S1-12), a constituent unit represented by the following formula (b-S1-12a) or (b-S1-12b) is particularly preferable.

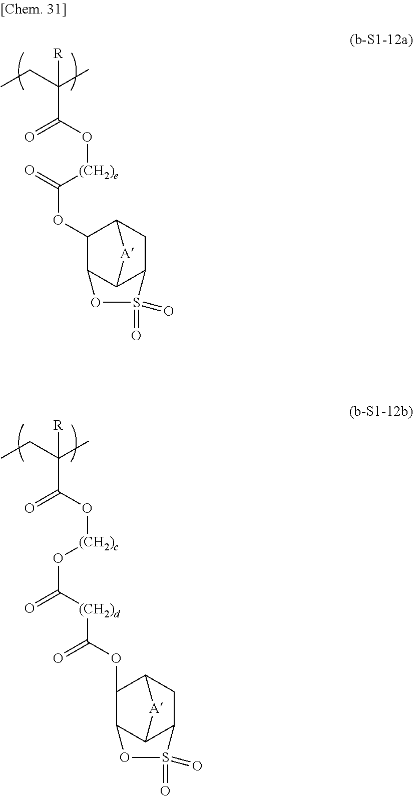

(In the formulae, R and A′ are each the same as the above, and c to e are each independently an integer of 1 or more and 3 or less.)
[Constituent unit (b-3-L)]

Examples of the constituent unit (b-3-L) include, for example, a constituent unit in which R^{11b} in the aforementioned formula (b-S1) is substituted with a lactone-containing cyclic group, and more specifically constituent units represented by the following formulae (b-L1) to (b-L5).

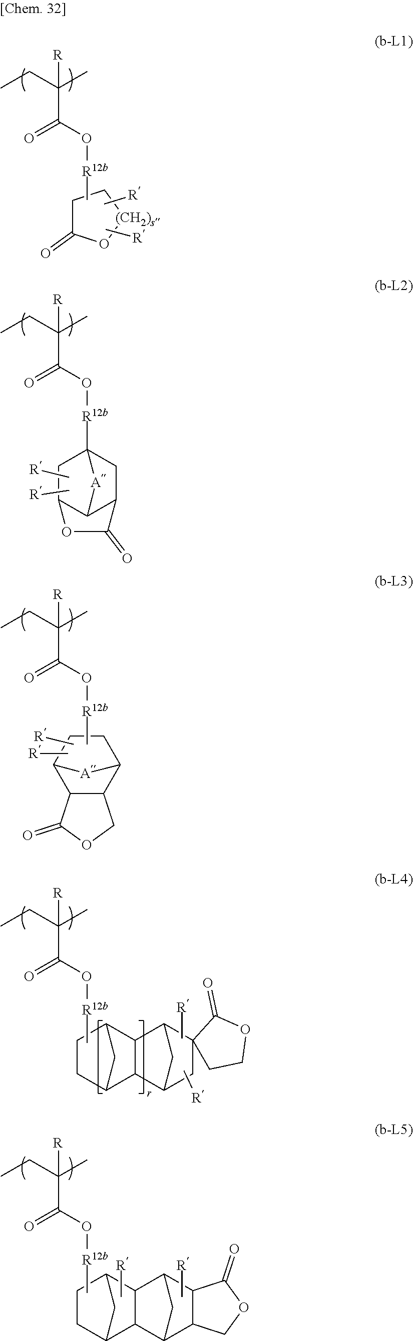

(In the formula, R represents a hydrogen atom, an alkyl group having 1 or more and 5 or less carbon atoms, or a halogenated alkyl group having 1 or more and 5 or less carbon atoms; R′ represents each independently a hydrogen atom, an alkyl group, an alkoxy group, a halogenated alkyl group, a hydroxyl group, —COOR″, —OC(═O)R″, a hydroxyalkyl group, or a cyano group, and R″ represents a hydrogen atom or an alkyl group; R^{12b} represents a single bond or divalent linking group, and s″ is an integer of 0 or more and 2 or less; A″ represents an alkylene group having 1 or more and 5 or less carbon atoms optionally including an oxygen atom or a sulfur atom, an oxygen atom or a sulfur atom; and r is 0 or 1.)

R in the formulae (b-L1) to (b-L5) is the same as the above. Examples of the alkyl group, the alkoxy group, the halogenated alkyl group, —COOR″, —OC(═O)R″, and the hydroxyalkyl group in R′ include groups the same as those described for the alkyl group, the alkoxy group, the halogenated alkyl group, —COOR″, —OC(═O)R″, and the hydroxyalkyl group recited as a substituent which the —SO₂— containing cyclic group may have, respectively.

R′ is preferably a hydrogen atom in view of easy industrial availability and the like. The alkyl group in R″ may be any of a linear, branched, or cyclic chain. In a case where R″ is a linear or branched alkyl group, the number of carbon atoms is preferably 1 or more and 10 or less, and more preferably 1 or more and 5 or less. In a case where R″ is a cyclic alkyl group, the number of carbon atoms is preferably 3 or more and 15 or less, more preferably 4 or more and 12 or less, and most preferably 5 or more and 10 or less. Specific examples include a group in which one or more hydrogen atoms are removed from monocycloalkane and polycycloalkane such as bicycloalkane, tricycloalkane, tetracycloalkane, and the like optionally substituted with a fluorine atom or a fluorinated alkyl group. Specific examples include a group in which one or more hydrogen atoms are removed from monocycloalkane such as cyclopentane and cyclohexane; and polycycloalkane such as adamantane, norbornane, isobornane, tricyclodecane, and tetracyclododecane; and the like. Examples of A″ include those the same as A′ in the aforementioned formula (3-1). A″ is preferably an alkylene group having 1 to 5 carbon atoms, an oxygen atom (—O—) or a sulfur atom (—S—), more preferably an alkylene group having 1 or more and 5 or less carbon atoms or —O—. As the alkylene group having 1 or more and 5 or less carbon atoms, a methylene group or a dimethylmethylene group is more preferable, and a methylene group is most preferable.

R^{12b} is the same as R^{12b} in the aforementioned formula (b-S1). In the formula (b-L1), s″ is preferably 1 or 2. Below, specific examples of the constituent units represented by the aforementioned formulae (b-L1) to (b-L3) will be illustrated. In each of the following formulae, R^{α} represents a hydrogen atom, a methyl group, or a trifluoromethyl group.

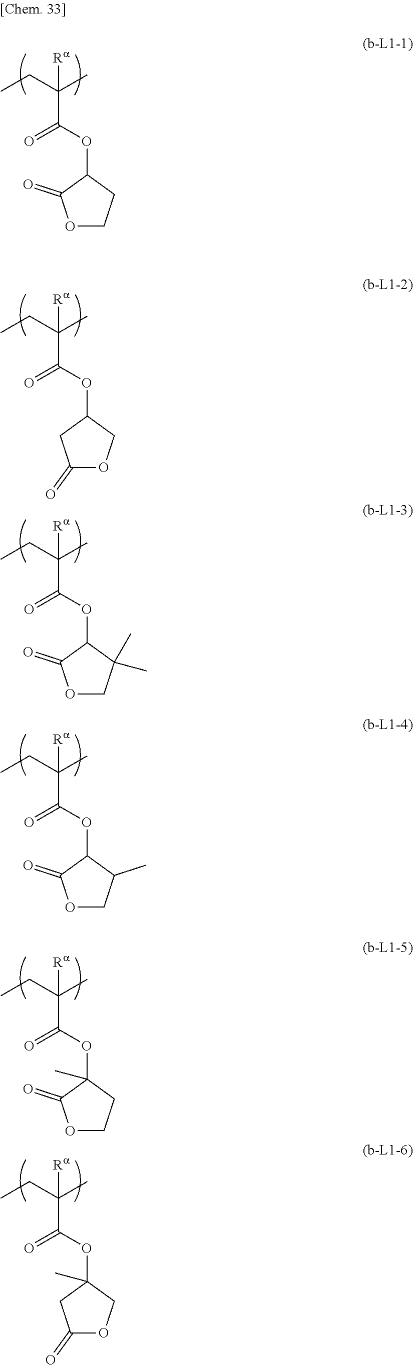

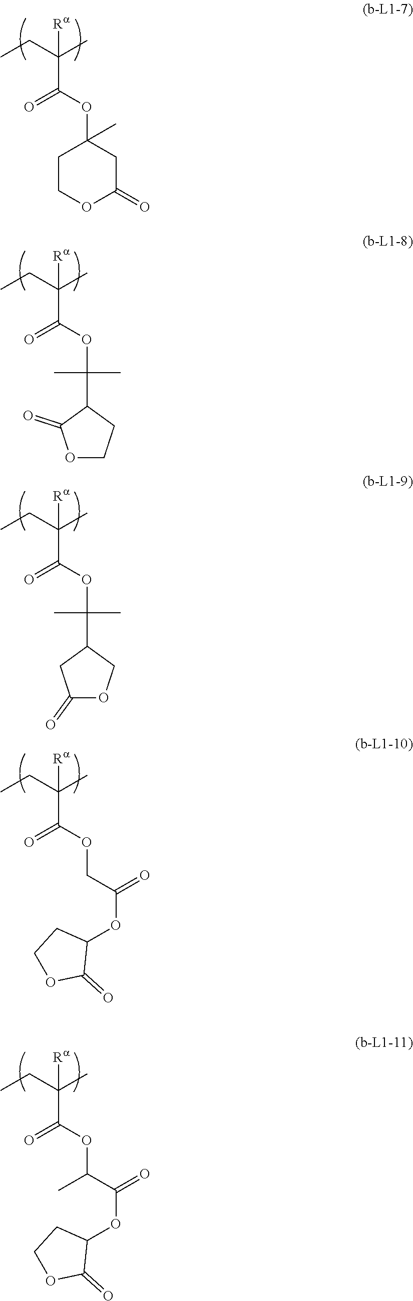

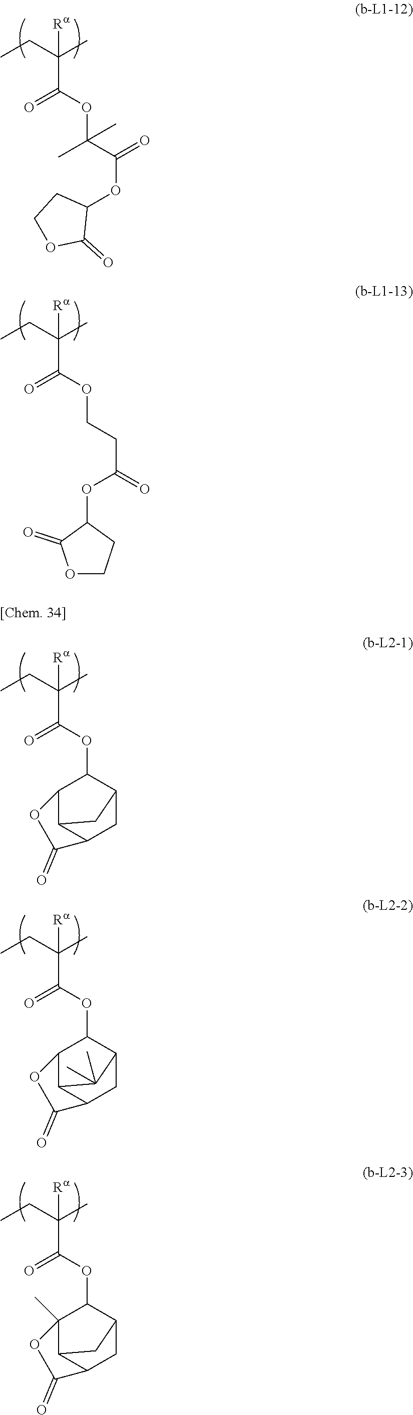

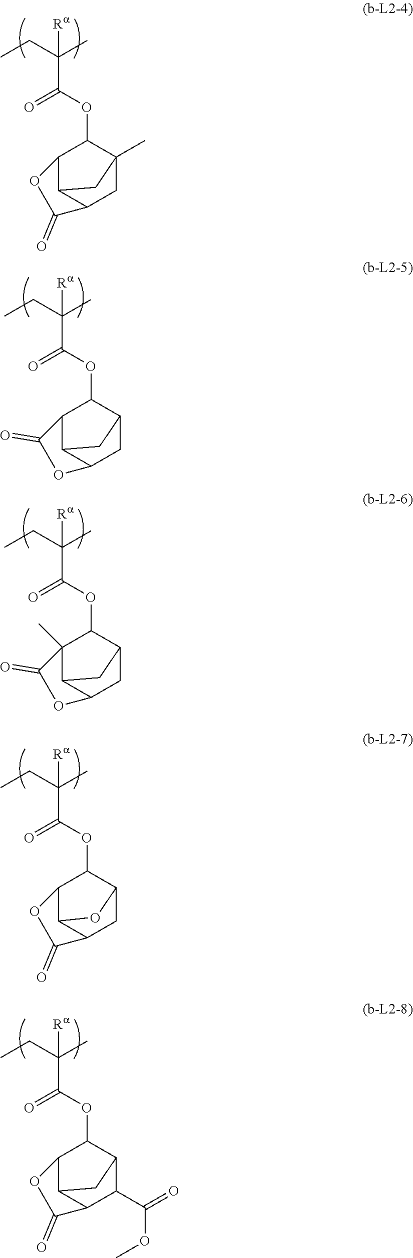

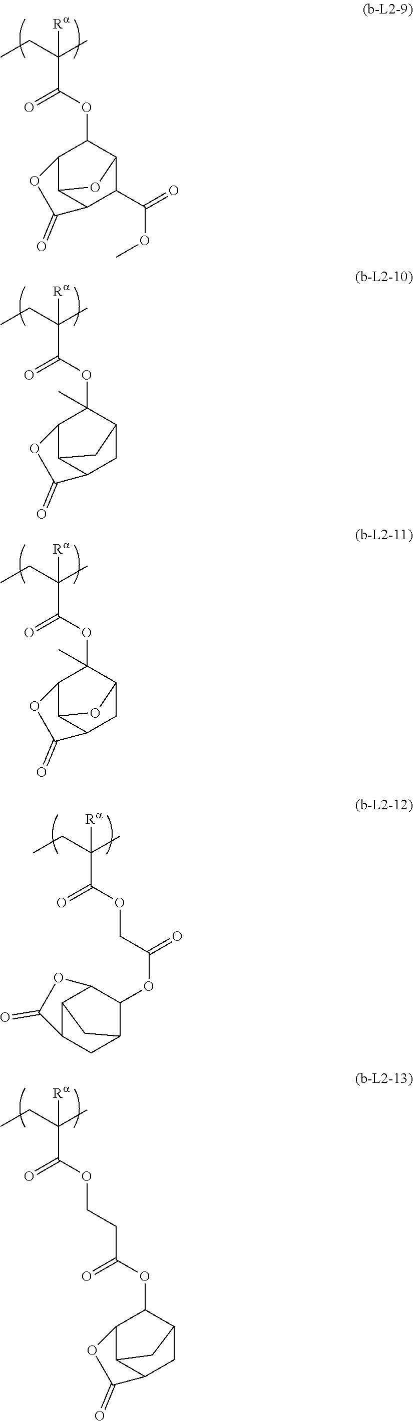

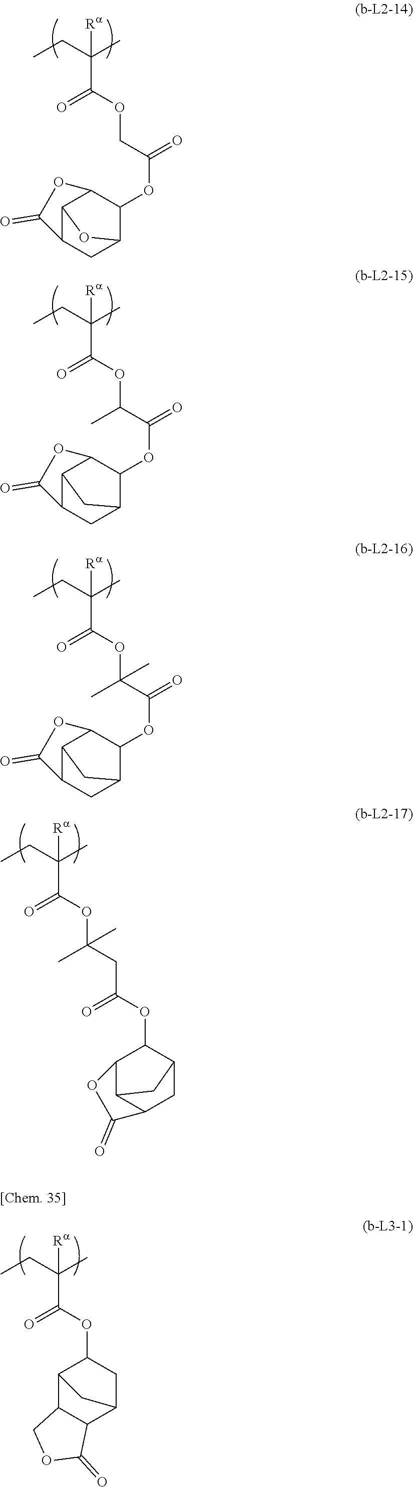

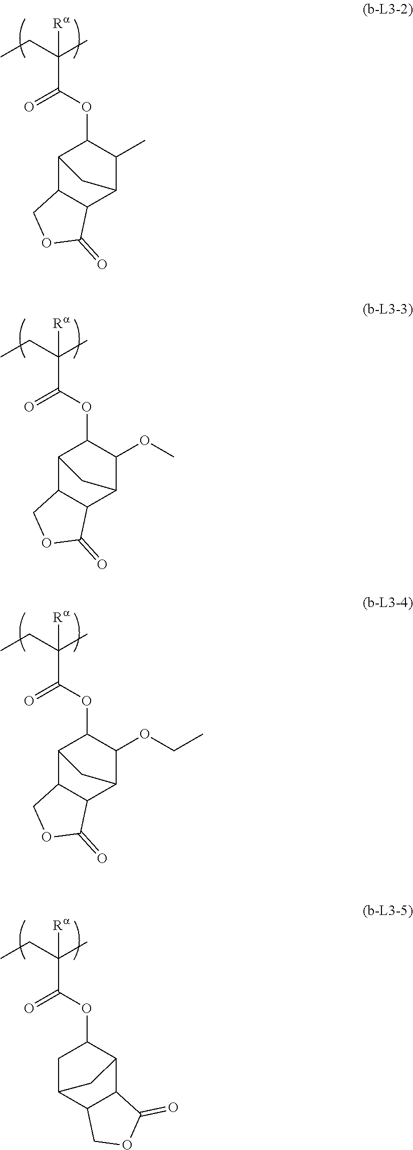

As the constituent unit (b-3-L), at least one constituent unit selected from the group consisting of the constituent units represented by the aforementioned formulae (b-L1) to (b-L5) is preferable, and at least one constituent unit selected from the group consisting of the constituent units represented by the formulae (b-L1) to (b-L3) is more preferable, and at least one constituent unit selected from the group consisting of the constituent units represented by the aforementioned formula (b-L1) or (b-L3) is particularly preferable. Among these, at least one selected constituent unit from the group consisting of the constituent units represented by the aforementioned formulae (b-L1-1), (b-L1-2), (b-L2-1), (b-L2-7), (b-L2-12), (b-L2-14), (b-L3-1) and (b-L3-5) is preferable.

Furthermore, as the constituent unit (b-3-L), the constituent units represented by following formulae (b-L6) to (b-L7) are also preferable.

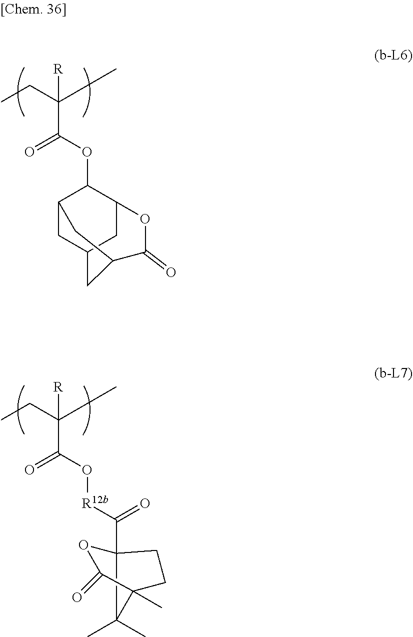

R and R^{12b} in the formulae (b-L6) and (b-L7) are the same as the above.

Furthermore, the acrylic resin (B3) includes constituent units represented by the following formulae (b5) to (b7), having an acid dissociable group, as constituent units that enhance the solubility of the acrylic resin (B3) in alkali under the action of acid.

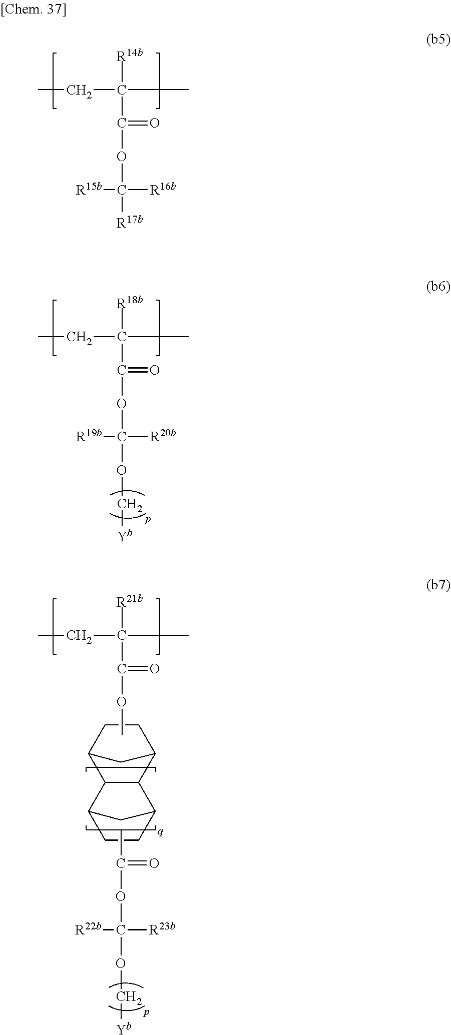

In the above formulae (b5) to (b7), R^{14b} and R^{18b} to R^{23b} each independently represents a hydrogen atom, a linear or branched alkyl group having 1 or more and 6 or less carbon atoms, a fluorine atom, or a linear or branched fluorinated alkyl group having 1 or more and 6 or less carbon atoms; R^{15b} to R^{17b} each independently represents a linear or branched alkyl group having 1 or more and 6 or less carbon atoms, a linear or branched fluorinated alkyl group having 1 or more and 6 or less carbon atoms, or an aliphatic cyclic group having 5 or more and 20 or less carbon atoms, and R^{16b} and R^{17b} may be bonded to each other to form a hydrocarbon ring having 5 or more and 20 or less carbon atoms together with carbon atoms to which both R^{16b} and R^{17b} are bonded; Y^{b} represents an optionally substituted aliphatic cyclic group or alkyl group; p is an integer of 0 or more and 4 or less; and q represents 0 or 1.

Note here that examples of the linear or branched alkyl group include a methyl group, an ethyl group, a propyl group, an isopropyl group, an n-butyl group, an isobutyl group, a tert-butyl group, a pentyl group, an isopentyl group, a neopentyl group, and the like. Furthermore, the fluorinated alkyl group refers to the abovementioned alkyl groups in which the hydrogen atoms are partially or entirely substituted with fluorine atoms. Specific examples of aliphatic cyclic groups include groups in which one or more hydrogen atoms are removed from monocycloalkanes or polycycloalkanes such as bicycloalkanes, tricycloalkanes, and tetracycloalkanes. Specifically, groups in which one hydrogen atom is removed from a monocycloalkane such as cyclopentane, cyclohexane, cycloheptane, or cyclooctane, or a polycycloalkane such as adamantane, norbornane, isobornane, tricyclodecane, or tetracyclododecane may be mentioned. In particular, groups in which one hydrogen atom is removed from cyclohexane or adamantane (which may further be substituted) are preferable.

When R^{16b} and R^{17b} are not bonded to each other to form a hydrocarbon ring, the above R^{15b}, R^{16b}, and R^{17b} are preferably a linear or branched alkyl group having 2 or more and 4 or less carbon atoms from the viewpoint of a high contrast and resolution, the depth of focus and the like, which are satisfactory. The above R^{19b}, R^{20b}, R^{22b} and R^{23b} are preferably a hydrogen atom or a methyl group.

The above R^{16b} and R^{17b} may form an aliphatic cyclic group having 5 or more and 20 or less carbon atoms together with a carbon atom to which the both R^{16b} and R^{17b} are bonded. Specific examples of such an alicyclic group include a group in which one or more hydrogen atoms are removed from monocycloalkanes, and polycycloalkanes such as bicycloalkanes, tricycloalkanes and tetracycloalkanes. Specific examples include a group in which one or more hydrogen atoms are removed from monocycloalkane such as cyclopentane, cyclohexane, cycloheptane, and cyclooctane, and polycycloalkane such as adamantane, norbornane, isobornane, tricyclodecane, and tetracyclododecane. In particular, groups in which one or more hydrogen atoms are removed from cyclohexane or adamantane (which may further be substituted) are preferable.

Further, in a case where an aliphatic cyclic group to be formed with the above R^{16b} and R^{17b} has a substituent on the ring skeleton thereof, examples of the substituent include a polar group such as a hydroxyl group, a carboxyl group, a cyano group, and an oxygen atom (═O), and a linear or branched alkyl group having 1 or more and 4 or less carbon atoms. As the polar group, an oxygen atom (═O) is particularly preferable.

The above Y^{b} is an alicyclic group or an alkyl group, and examples thereof include groups of monocycloalkanes and polycycloalkanes such as bicycloalkanes, tricycloalkanes, and tetracycloalkanes from which one or more hydrogen atoms are removed. Specific examples thereof are the groups of monocycloalkanes such as cyclopentane, cyclohexane, cycloheptane and cyclooctane, and polycycloalkanes such as adamantane, norbornane, isobornane, tricyclodecane and tetracyclododecane, from which one or more hydrogen atoms are removed. In particular, groups in which one or more hydrogen atoms are removed from cyclohexane or adamantane (which may further be substituted) are preferable.

Further, in a case where the alicyclic group of the above Y^{b} has a substituent on the ring skeleton thereof, examples of the substituent include a polar group such as a hydroxyl group, a carboxyl group, a cyano group and an oxygen atom (═O), and a linear or branched alkyl group having 1 or more and 4 or less carbon atoms. As the polar group, an oxygen atom (═O) is particularly preferable.

When Y^{b} is an alkyl group, it is preferably a linear or branched alkyl group having 1 or more and 20 or less carbon atoms, and more preferably 6 or more and 15 or less carbon atoms. The alkyl group is an alkoxyalkyl group particularly preferable. Examples of such an alkoxyalkyl group include a 1-methoxyethyl group, 1-ethoxyethyl group, 1-n-propoxyethyl group, 1-isopropoxyethyl group, 1-n-butoxyethyl group, 1-isobutoxyethyl group, 1-tert-butoxyethyl group, 1-methoxypropyl group, 1-ethoxypropyl group, 1-methoxy-1-methylethyl group, 1-ethoxy-1-methylethyl group, and the like.

Preferable specific examples of the constituent unit represented by the above formula (b5) include those represented by the following formulae (b5-1) to (b5-33).

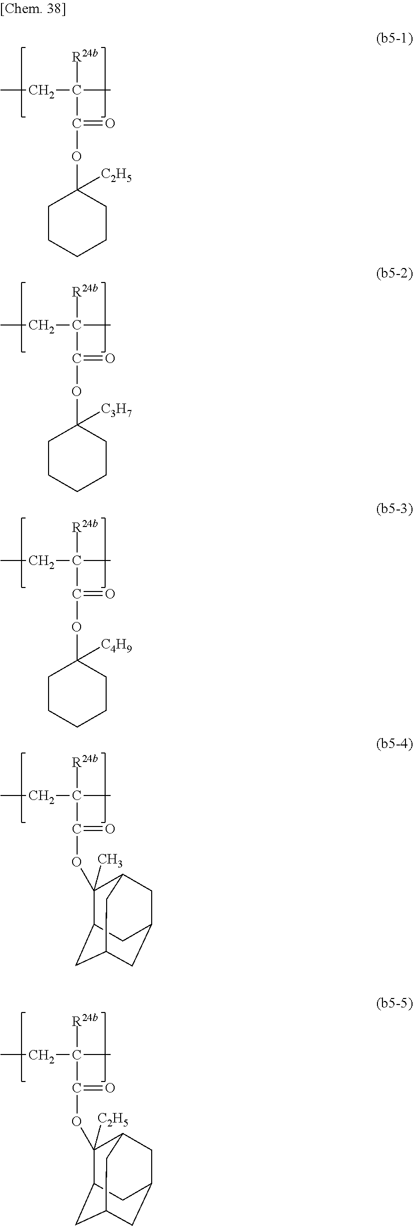

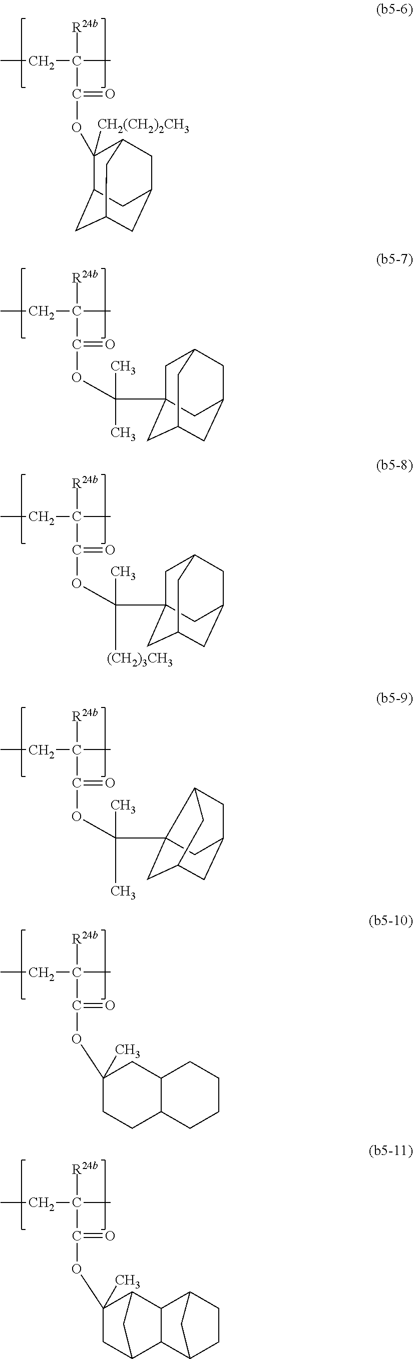

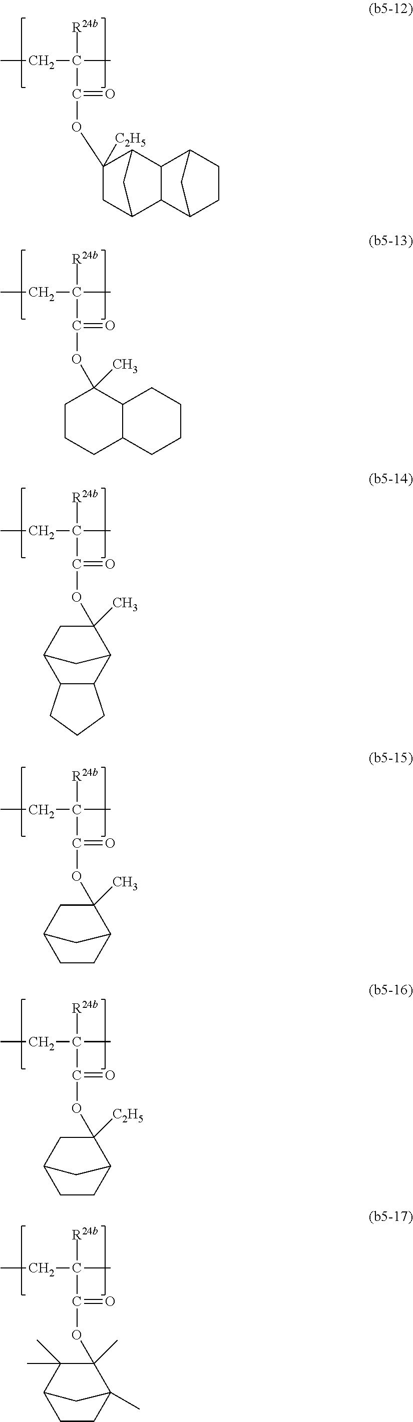

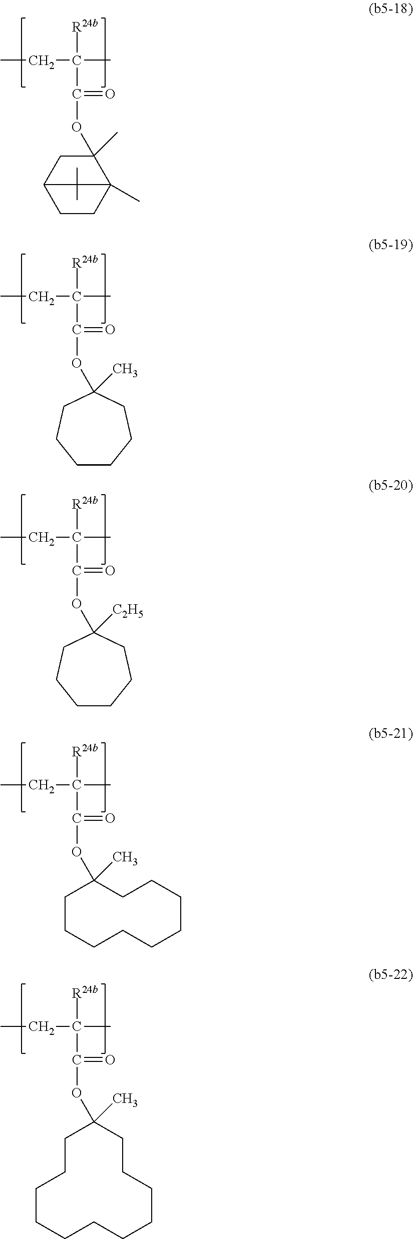

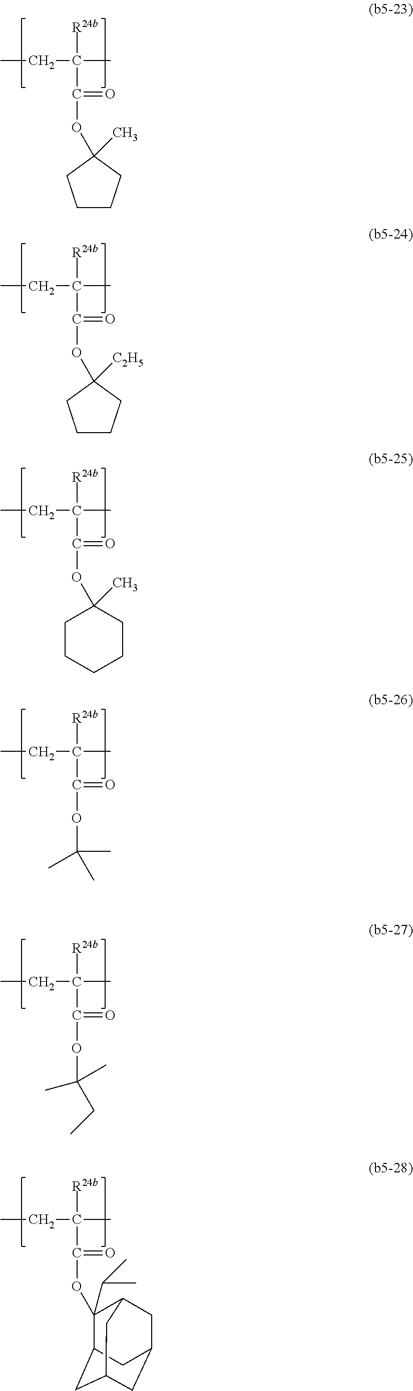

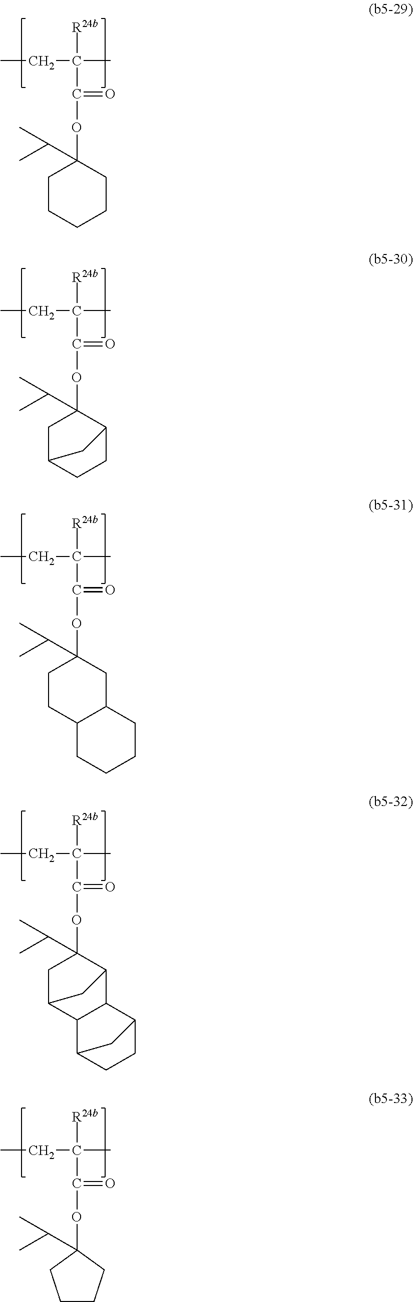

In the above formulae (b5-1) to (b5-33), R^{24b} represents a hydrogen atom or a methyl group.

Preferable specific examples of the constituent unit represented by the above formula (b6) include constituent units represented by the following formulae (b6-1) to (b6-26).

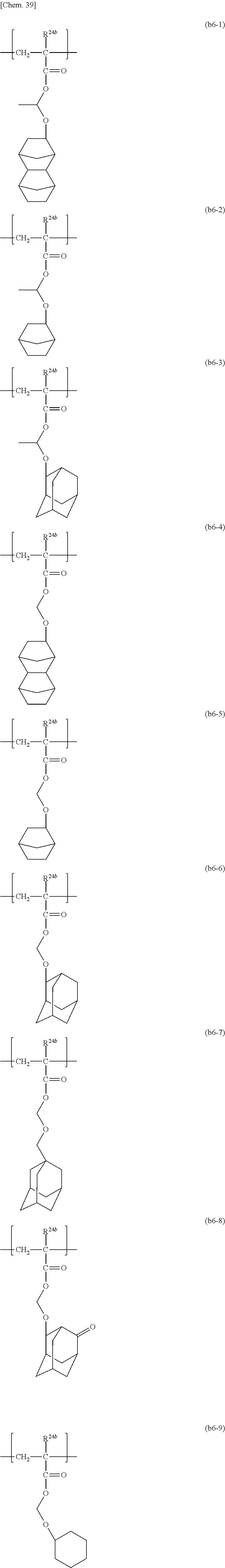

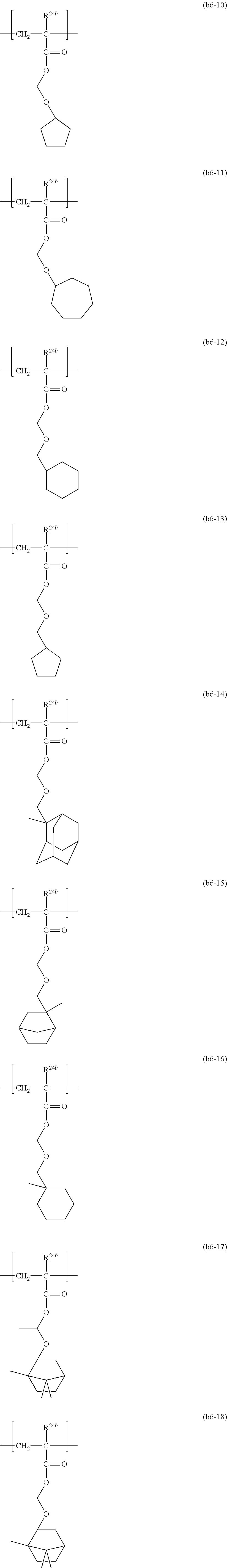

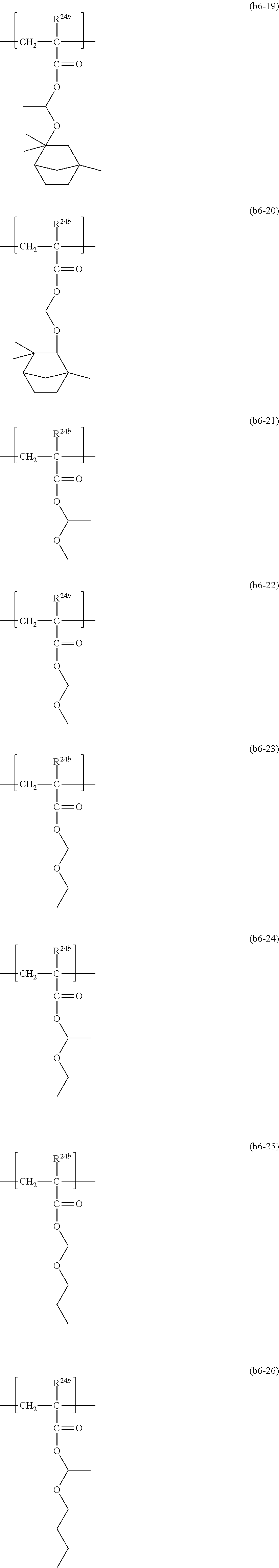

In the above formulae (b6-1) to (b6-26), R^{24b} represents a hydrogen atom or a methyl group.

Preferable specific examples of the constituent unit represented by the above formula (b7) include constituent units represented by the following formulae (b7-1) to (b7-15).

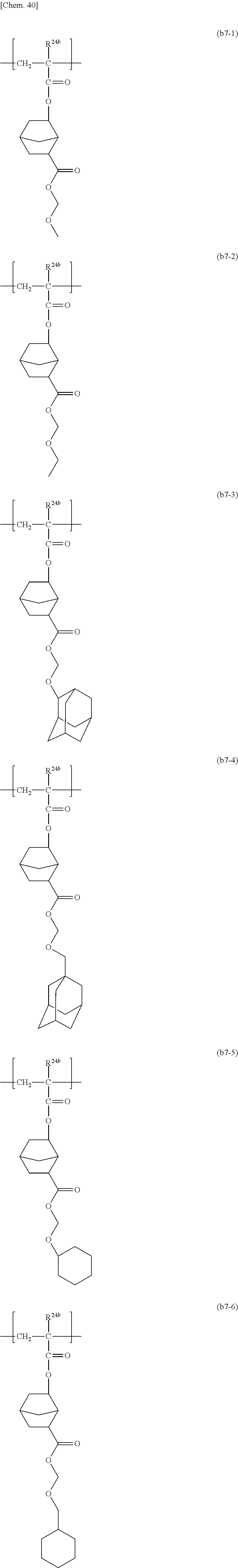

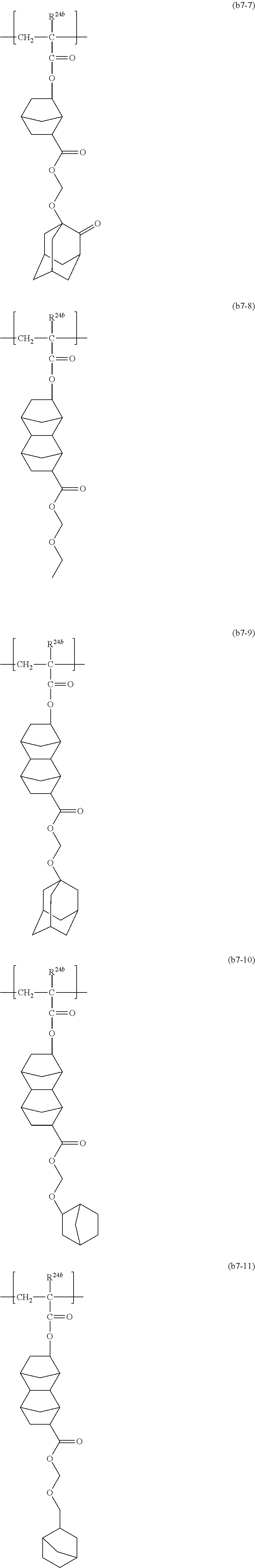

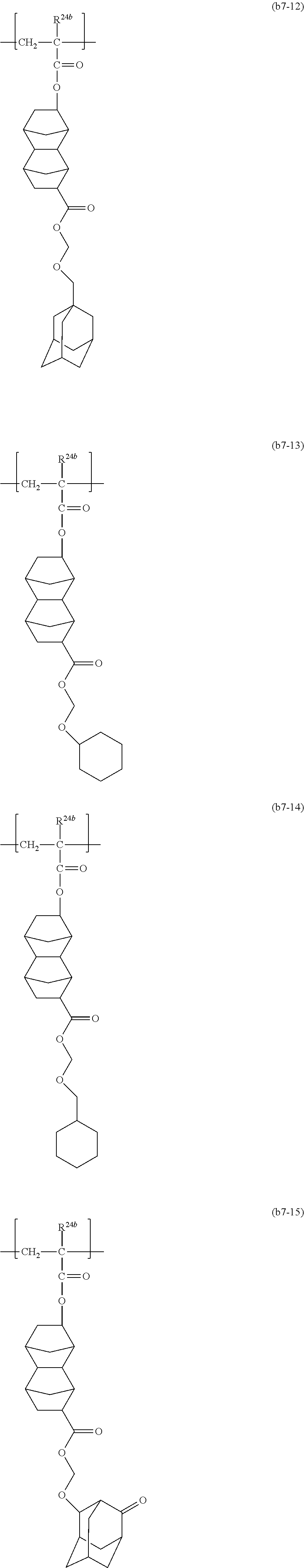

In the above formulae (b7-1) to (b7-15), R^{24b} represents a hydrogen atom or a methyl group.

Among the constituent units represented by the formulae (b5) to (b7) described above, the structural unit represented by formula (b6) is preferable because it can be easily synthesized and made to have relatively high sensitivity. Further, among the constituent units represented by the formula (b6), those in which Y^{b} is an alkyl group are preferable, and those in which one or both of R^{19b} and R^{20b} are alkyl groups are preferable.

Furthermore, the acrylic resin (B3) is preferably a resin including a copolymer including a constituent unit derived from a polymerizable compound having an ether bond together with the constituent unit represented by the above formulae (b5) to (b7).

Examples of the polymerizable compound having an ether bond include radical polymerizable compounds such as (meth)acrylic acid derivatives having an ether bond and an ester bond, and specific examples thereof include 2-methoxyethyl (meth)acrylate, 2-ethoxyethyl (meth)acrylate, methoxytriethylene glycol (meth) acrylate, 3-methoxybutyl (meth) acrylate, ethylcarbitol (meth) acrylate, phenoxypolyethylene glycol (meth) acrylate, methoxypolyethylene glycol (meth)acrylate, methoxypolypropylene glycol (meth)acrylate, tetrahydrofurfuryl (meth)acrylate, and the like. Also, the above polymerizable compound having an ether bond is preferably, 2-methoxyethyl (meth)acrylate, 2-ethoxyethyl (meth)acrylate, or methoxytriethylene glycol (meth)acrylate. These polymerizable compounds may be used alone, or in combinations of two or more thereof.

Furthermore, the acrylic resin (B3) may include another polymerizable compound as a constituent unit in order to moderately control physical or chemical properties. The polymerizable compound is exemplified by conventional radical polymerizable compounds and anion polymerizable compounds.

Examples of such a polymerizable compound include monocarboxylic acids such as acrylic acid, methacrylic acid, and crotonic acid; dicarboxylic acids such as maleic acid, fumaric acid, and itaconic acid; methacrylic acid derivatives having a carboxyl group and an ester bond such as 2-methacryloyloxyethyl succinic acid, 2-methacryloyloxyethyl maleic acid, 2-methacryloyloxyethyl phthalic acid, and 2-methacryloyloxyethyl hexahydrophthalic acid; (meth)acrylic acid alkyl esters such as methyl(meth)acrylate, ethyl(meth)acrylate, butyl(meth)acrylate and cyclohexyl(meth)acrylate; (meth)acrylic acid hydroxyalkyl esters such as 2-hydroxyethyl (meth)acrylate and 2-hydroxypropyl (meth)acrylate; (meth)acrylic acid aryl esters such as phenyl (meth)acrylate and benzyl (meth)acrylate; dicarboxylic acid diesters such as diethyl maleate and dibutyl fumarate; vinyl group-containing aromatic compounds such as styrene, α-methylstyrene, chlorostyrene, chloromethylstyrene, vinyltoluene, hydroxystyrene, α-methylhydroxystyrene, and α-ethylhydroxystyrene; vinyl group-containing aliphatic compounds such as vinyl acetate; conjugated diolefins such as butadiene and isoprene; nitrile group-containing polymerizable compounds such as acrylonitrile and methacrylonitrile; chlorine-containing polymerizable compounds such as vinyl chloride and vinylidene chloride; amide bond-containing polymerizable compounds such as acrylamide and methacrylamide; and the like.

As described above, the acrylic resin (B3) may include a constituent unit derived from a polymerizable compound having a carboxy group such as the above monocarboxylic acids and dicarboxylic acids. However, it is preferable that the acrylic resin (B3) does not substantially include a constituent unit derived from a polymerizable compound having a carboxyl group, since a resist pattern including a nonresist portion having a favorable rectangular cross-sectional shape can easily be formed. Specifically, the proportion of a constituent unit derived from a polymerizable compound having a carboxyl group in the acrylic resin (B3) is preferably 20% by mass or less, more preferably 15% by mass or less, and particularly preferably 5% by mass or less. In acrylic resin (B3), acrylic resin including a relatively large amount of constituent unit derived from a polymerizable compound having a carboxy group is preferably used in combination with an acrylic resin that includes only a small amount of constituent unit derived from a polymerizable compound having a carboxy group or does not include this constituent unit.

Furthermore, examples of the polymerizable compound include (meth)acrylic acid esters having a non-acid-dissociable aliphatic polycyclic group, and vinyl group-containing aromatic compounds and the like. As the non-acid-dissociable aliphatic polycyclic group, particularly, a tricyclodecanyl group, an adamantyl group, a tetracyclododecanyl group, an isobornyl group, a norbornyl group, and the like are preferred in view of easy industrial availability and the like. These aliphatic polycyclic groups may have a linear or branched alkyl group having 1 or more and 5 or less carbon atoms as a substituent.

Specific examples of the constituent units derived from the (meth)acrylic acid esters having a non-acid-dissociable aliphatic polycyclic group include those having structures represented by the following formulae (b8-1) to (b8-5).

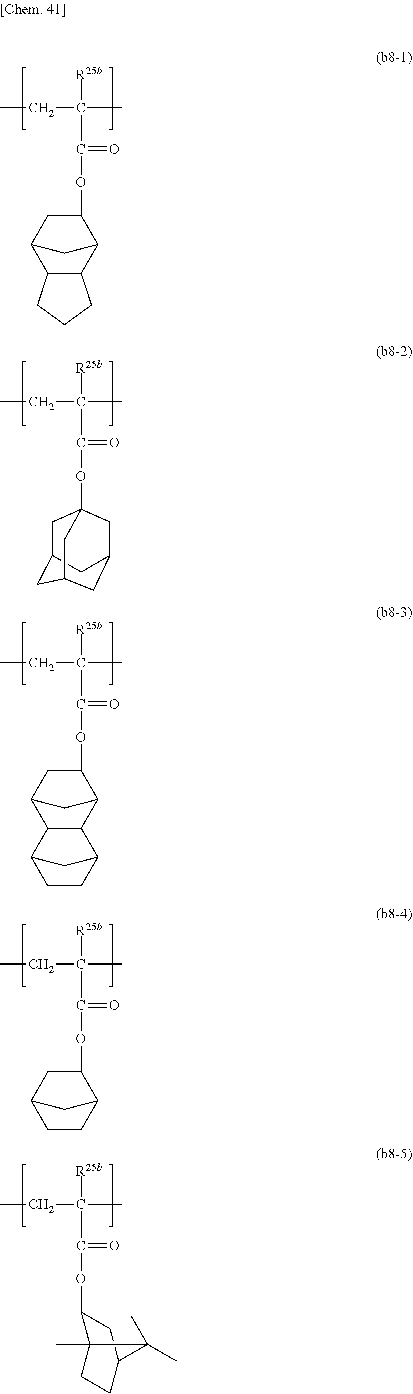

In the formulae (b8-1) to (b8-5), R^{25b} represents a hydrogen atom or a methyl group.

When the acrylic resin (B3) includes the constituent unit (b-3) including an —SO₂-containing cyclic group or a lactone-containing cyclic group, the content of the constituent unit (b-3) in the acrylic resin (B3) is preferably 5% by mass or more, more preferably 10% by mass or more, and particularly preferably 10% by mass or more and 50% by mass or less, and most preferably 10% by mass or more and 30% by mass or less. In a case where the photosensitive resin composition includes the constituent unit (b-3) having the above-mentioned range of amount, both favorable developing property and a favorable pattern shape can be easily achieved simultaneously.

Furthermore, in the acrylic resin (B3), a constituent unit represented by the aforementioned formulae (b5) to (b7) is preferably included in an amount of 5% by mass or more, more preferably 10% by mass or more, and particularly preferably 10% by mass or more and 50% by mass or less.

The acrylic resin (B3) preferably includes the above constituent unit derived from a polymerizable compound having an ether bond. The content of the constituent unit derived from a polymerizable compound having an ether bond in the acrylic resin (B3) is preferably 0% by mass or more and 50% by mass or less, and more preferably 5% by mass or more and 30% by mass or less.

The acrylic resin (B3) preferably includes the above constituent unit derived from (meth)acrylic acid esters having a non-acid-dissociable aliphatic polycyclic group. The content of the constituent unit derived from (meth)acrylic acid esters having a non-acid-dissociable aliphatic polycyclic group in the acrylic resin (B3) is preferably 0% by mass or more and 50% by mass or less, and more preferably 5% by mass or more and 30% by mass or less.

As long as the photosensitive resin composition contains a predetermined amount of the acrylic resin (B3), an acrylic resin other than the acrylic resin (B3) described above can also be used as the resin (B). Such an acrylic resin other than the acrylic resin (B3) is not particularly limited as long as it includes a constituent unit represented by the aforementioned formulae (b5) to (b7).

The mass-average molecular weight of the resin (B) described above in terms of polystyrene is preferably 10000 or more and 600000 or less, more preferably 20000 or more and 400000 or less, and even more preferably 30000 or more and 300000 or less. A mass-average molecular weight within these ranges allows a photosensitive layer to maintain sufficient strength without lowering detachability from a substrate, and can further prevent swelling of a profile and crack generation at the time of plating.

It is also preferred that the resin (B) has a dispersivity of 1.05 or more. Dispersivity herein indicates a value of a mass average molecular weight divided by a number average molecular weight. Such a dispersivity can avoid problems with respect to stress resistance on intended plating or possible swelling of metal layers resulting from the plating treatment.

The content of the resin (B) is preferably 5% by mass or more and 70% by mass or less with respect to the total solid content of the photosensitive resin composition.

### <Acid Diffusion Suppressing Agent (C)>

An acid diffusion suppressing agent (C) included in a photosensitive resin composition includes a compound represented by the following formula (c1), which is decomposed by irradiation with active rays or radiation. When as the acid diffusion suppressing agent (C), the compound represented by the following formula (c1), which is decomposed by irradiation with active rays or radiation, is blended in the photosensitive resin composition, an effect that the photosensitive resin composition becomes a photosensitive resin composition from which a resist pattern having a rectangular cross-sectional shape is easily formed by a photoreactive acid diffusion suppressing agent is exhibited, and decomposition by the acid diffusion suppressing agent of the non-ionic acid generating agent can be suppressed.

In detail, the photoreactive acid diffusion suppressing agent has a quenching power of trapping acid generated from the acid generating agent upon irradiation (exposure) with the active rays or radiation through the ion exchange reaction. Since the photodecomposition-type acid diffusion suppressing agent is decomposed and loses the quenching power when exposed to light, the photodecomposition-type acid diffusion suppressing agent acts as the acid diffusion suppressing agent in an unexposed portion, and does not act as the acid diffusion suppressing agent in an exposed portion. Therefore, use of the photodecomposition-type acid diffusion suppressing agent makes it possible to suppress diffusion of the acid from the exposed portion to the unexposed portion. Therefore, the acid concentration contrast between the exposed portion and the unexposed portion can be improved, and the lithography property such as a shape can be improved. However, the present inventors have found that when the general photoreactive acid diffusion suppressing agent is used together with the above-described non-ionic acid generating agent, a problem that the non-ionic acid generating agent is decomposed easily occurs. In the photosensitive resin composition described above, a compound represented by the formula (c1) as the photodecomposition-type acid suppressing agent, which is decomposed by irradiation with active rays or radiation, is used as the acid diffusion suppressing agent (C), decomposition of the non-ionic acid generating agent can be suppressed.

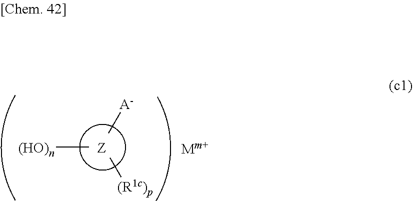

(In the formula (c1),
M^{m+} represents an m-valent organic cation,
m represents an integer of 1 or more,
a ring Z represents a benzene ring, or a benzene ring-fused polycyclic,
the number x of benzene rings of the ring Z represents an integer of 1 or more and 4 or less,
R^{1c} represents a substituent,
A⁻ represents —COO⁻ or —SO₂O⁻,
n represents an integer of 2 or more and 2x+3 or less,
p represents an integer of 0 or more and 2x+3-n or less, and
when p is 2 or more, a plurality of R^{1c}s may be the same as or different from each other, and a plurality of R^{1c}s may be linked to each other to form a ring).

In the formula (c1), the ring Z represents a benzene ring, or a polycyclic in which 2 or more and 4 or less benzene rings are fused. The number x of the benzene rings of the ring Z is an integer of 1 or more and 4 or less. Examples of the polycyclic in which 2 or more and 4 or less benzene rings are fused include a naphthalene ring, an anthracene ring, a phenanthrene ring, a tetracene ring.

In the formula (c1), A⁻ is —COO⁻ or —SO₂O⁻. In a case where A⁻ is —COO⁻, when the compound represented by the formula (c1) is decomposed by irradiation with active rays or radiation, carboxylic acid is generated. Furthermore, when A⁻ is —SO₂O⁻, the compound represented by the formula (c1) is decomposed by irradiation with active rays or radiation, sulfonic acid is generated.

In the formula (c1), examples of the substituent as R^{1c} include a halogen atom, an alkyl group having 1 or more and 5 or less carbon atoms, an alkoxy group having 1 or more and 5 or less carbon atoms, or a fluoroalkyl group having 1 or more and 5 or less carbon atoms. Examples of the halogen atom includes a fluorine atom, a chlorine atom, a bromine atom, and an iodine atom. The alkyl group having 1 or more and 5 or less carbon atoms may be linear or branched, or cyclic. The alkyl group may include a divalent group including a hetero atom such as —O—, —COO—, and —OCO— in a chain thereof, and may include a substituent such as a hydroxy group. Specific examples of the alkyl group include a methyl group, an ethyl group, an n-propyl group, an isopropyl group, an n-butyl group, an isobutyl group, a sec-butyl group, a tert-butyl group, an n-pentyl group, and the like. The alkoxy group having 1 or more and 5 or less carbon atoms may be linear or branched, or cyclic. Specific examples of the alkoxy group include a methoxy group, an ethoxy group, an n-propyloxy group, an isopropyloxy group, an n-butyloxy group, an isobutyloxy group, a sec-butyloxy group, a tert-butyloxy group, and an n-pentyloxy group. Examples of the fluoroalkyl group having 1 or more and 5 or less carbon atoms include a group in which a part or all of the hydrogen atoms of the alkyl group mentioned as an alkyl group as a substituent are substituted with the aforementioned halogen atom. When p is 2 or more, a plurality of R^{1c}s may be the same as or different from each other. The plurality of R^{1c}s may be linked to each other to form a ring. For example, two R^{1c} may be bonded to adjacent carbon atoms of ring Z, respectively, to form a ring together with the bonding carbon atoms. Examples of the ring to be formed may include a tetrahydropyran ring, a dihydropyran ring, and a lactone ring, which may have a substituent such as a hydroxy group or an alkyl group.

In the ring Z, bonding positions of A⁻, the hydroxy group, and R^{1c} are not particularly limited. For example, the bonding position of the hydroxy group with respect to the bonding position of A⁻ in the benzene ring to which A⁻ is bonded may be any of the ortho, meta, and para positions. From the viewpoint of stability of the photosensitive resin composition, it is preferable that the bonding position of at least one hydroxy group with respect to the bonding position of A⁻ in the benzene ring to which A⁻ is bonded is an ortho position, in other words, that the hydroxyl group is bonded to at least one of two carbon atoms adjacent to a carbon atom to which the A⁻ is bonded in the ring Z.

In the formula (c1), n represents an integer of 2 or more and 2x+3 or less, preferably 4 or less, and more preferably 3 or less.

Specific examples of the anionic portion in the compound represented by the formula (c1) include the following anions.

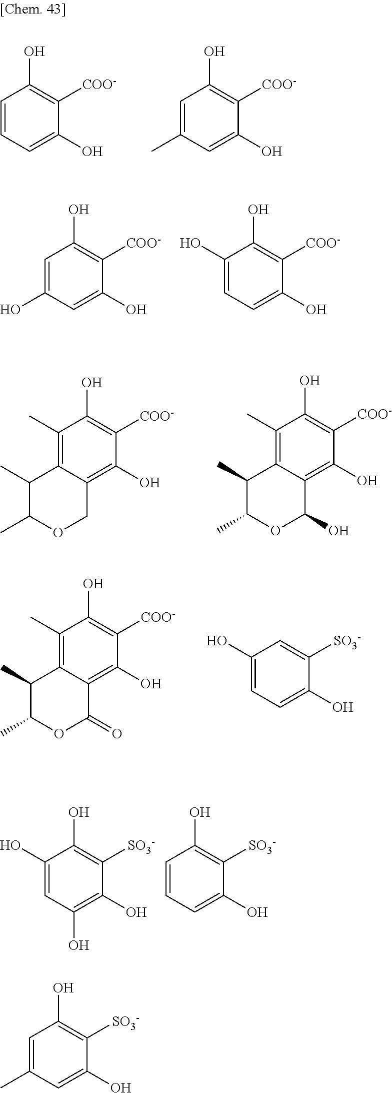

The m-valent organic cation as M^{m+} is not particularly limited, and may be any cation that can form a salt with the anion in the formula (c1).

Examples of the m-valent organic cation as M^{m+} include a cation represented by the following formula (c1-1).

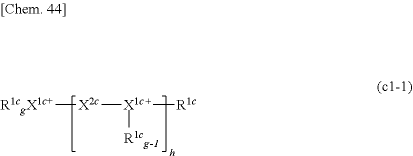

In the formula (c1-1), X^{1c} represents a sulfur atom or iodine atom respectively having a valence of g; g represents 1 or 2. h represents the number of repeating units in the structure within parentheses. R^{1c} represents an organic group that is bonded to X^{1c}, and represents an aryl group having 6 or more and 30 or less carbon atoms, a heterocyclic group having 4 or more and 30 or less carbon atoms, an alkyl group having 1 or more and 30 or less carbon atoms, an alkenyl group having 2 or more and 30 or less carbon atoms, or an alkynyl group having 2 or more and 30 or less carbon atoms, and R^{1c} may be substituted with at least one group selected from the group consisting of an alkyl group, a hydroxyl group, an alkoxy group, an alkylcarbonyl group, an arylcarbonyl group, an alkoxycarbonyl group, an aryloxycarbonyl group, an arylthiocarbonyl group, an acyloxy group, an arylthio group, an alkylthio group, an aryl group, a heterocyclic group, an aryloxy group, an alkylsulfinyl group, an arylsulfinyl group, an alkylsulfonyl group, an arylsulfonyl group, an alkyleneoxy group, an amino group, a cyano group, a nitro group, and halogen atoms. The number of R^{1c}s is g+h (g−1)+1, and the R^{1c}s may be respectively identical to or different from each other. Furthermore, two or more R^{1c}s may be bonded to each other directly or via —O—, —S—, —SO—, —SO₂—, —NH—, —NR^{2a}—, —CO—, —COO—, —CONH—, an alkylene group having 1 or more and 3 or less carbon atoms, or a phenylene group, and may form a ring structure including X^{1c}. R^{2c} represents an alkyl group having 1 or more and 5 or less carbon atoms, or an aryl group having 6 or more and 10 or less carbon atoms.

X^{2c} represents a structure represented by the following formula (c1-2).

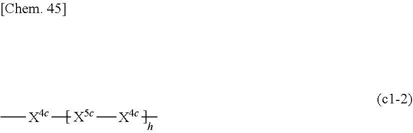

In the above formula (c1-2), X⁴c represents an alkylene group having 1 or more and 8 or less carbon atoms, an arylene group having 6 or more and 20 or less carbon atoms, or a divalent group of a heterocyclic compound having 8 or more and 20 or less carbon atoms, and X⁴c may be substituted with at least one selected from the group consisting of an alkyl group having 1 or more and 8 or less carbon atoms, an alkoxy group having 1 or more and 8 or less carbon atoms, an aryl group having 6 or more and 10 or less carbon atoms, a hydroxyl group, a cyano group, a nitro group, and halogen. X⁵c represents —O—, —S—, —SO—, —SO₂—, —NH—, —NR^{2c}—, —CO—, —COO—, —CONH—, an alkylene group having 1 or more and 3 or less carbon atoms, or a phenylene group. h represents the number of repeating units of the structure in parentheses. X^{4c}s in the number of h+1 and X^{5c}s in the number of h may be the same as or different from each other. R^{2c} has the same definition as described above.

Examples of the cation represented by the above formula (c1-1) include triphenylsulfonium, tri-p-tolylsulfonium, 4 (phenylthio)phenyldiphenylsulfonium, bis[4-(diphenylsulfonio)phenyl] sulfide, bis[4-{bis[4-(2-hydroxyethoxy)phenyl]sulfonio}phenyl] sulfide, bis{4-[bis(4-fluorophenyl)sulfonio]phenyl} sulfide, 4-(4-benzoyl-2-chlorophenylthio)phenylbis(4-fluorophenyl)sulfonium, 7-isopropyl-9-oxo-10-thia-9,10-dihydroanthracen-2-yldi-p-tolylsulfonium, 7-isopropyl-9-oxo-10-thia-9,10-dihydroanthracen-2-yldiphenylsulfonium, 2-[(diphenyl)sulfonio]thioxanthone, 4-[4-(4-tert-butylbenzoyl)phenylthio]phenyldi-p-tolylsulfonium, 4-(4-benzoylphenylthio)phenyldiphenylsulfonium, diphenylphenacylsulfonium, 4-hydroxyphenylmethylbenzylsulfo-nium, 2-naphthylmethyl(1-ethoxycarbonyl)ethylsulfonium, 4-hydroxyphenylmethylphenacylsulfonium, phenyl[4-(4-biphenylthio)phenyl]-4-biphenylsulfonium, phenyl[4-(4-biphenylthio)phenyl]-3-biphenylsulfonium, [4-(4-acetophenylthio) phenyl] diphenylsulfonium, octadecylmethylphenacylsulfonium, diphenyliodonium, di-p-tolyliodonium, bis(4-dodecylphenyl)iodonium, bis(4-methoxyphenyl)iodonium, (4-octyloxyphenyl)phenyliodonium, bis(4-decyloxy)phenyliodonium, 4-(2-hydroxytetradecyloxy)phenylphenyliodonium, 4-isopropylphenyl(p-tolyl)iodonium, 4-isobutylphenyl(p-tolyl)iodonium, or the like.

Among the cations represented by the above formula (c1-1), a preferable cation may be a cation represented by the following formula (c1-3).

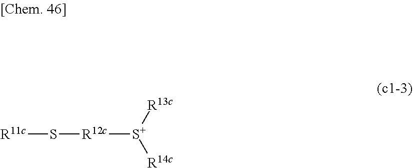

In the above formula (c1-3), R^{11c}, R^{13c}, and R^{14c} each independently represents an aryl group which may have a substituent, and R^{12c} represents an arylene group which may have a substituent.

In the above formula (c1-3), examples of the aryl group as R^{11c}, R^{13c}, and R^{14c} include a phenyl group, a naphthyl group, an anthracenyl group, a phenanthryl group, a biphenyly group, and a fluorenyl group. Examples of the arylene group as R^{12c} include a group in which one hydrogen atom is removed from the aryl group as R^{11c}, R^{13c}, and R^{14c}. Examples of the substituent which the aryl group as R^{11c}, R^{13c}, and R^{14c} may have or the substituent which the arylene group as R^{12c} may have include alkyl, hydroxy, alkoxy, alkylcarbonyl, alkylcarbonyloxy, alkyloxycarbonyl, and a halogen atom. As R^{12c}, a p-phenylene group, or an m-phenylene group is preferable, and a p-phenylene group is more preferable. As R^{11c}, R^{13c}, and R^{14c}, each independently, a phenyl group which may have a substituent, a naphthyl group which may have a substituent, a biphenylyl group which may have a substituent, a fluorenyl group which may have a substituent are preferable, a phenyl group, a fluorene-2-yl group, or a 9,9-dimethyl fluorene-2-yl group is more preferable.

Specific examples of the sulfonium ion represented by the above formula (c1-3) include 4-(phenylthio)phenyldiphenylsulfonium, 4-(4-benzoyl-2-chlorophenylthio)phenylbis(4-fluorophenyl)sulfonium, 4-(4-benzoylphenylthio)phenyldiphenylsulfonium, phenyl[4-(4-biphenylthio)phenyl]-4-biphenylsulfonium, phenyl[4-(4-biphenylthio)phenyl]-3-biphenylsulfonium, [4-(4-acetophenylthio)phenyl]diphenylsulfonium, diphenyl[4-(p-terphenylthio)phenyl]diphenylsulfonium, and phenyl[4-(9,9-dimethylfluorene-2-ylthio)phenyl]9,9-dimethylfluorene-2-ylsulfonium.

In the formula (c1), m is preferably 1.

The compound represented by the formula (c1) is preferably a compound represented by the following formula (c2).

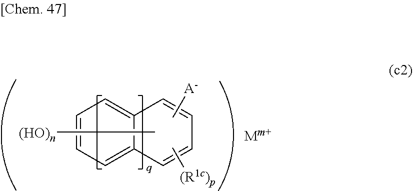

(In the formula (c2), M^{m+}, R^{1c}, A⁻, m, n, and p are respectively the same as those in the formula (c1), q represents an integer of 0 or more and 3 or less, n, p, and q satisfy a formula, n+p≤(q×2)+5.)

The compound represented by the formula (c1) can be produced by well-known methods. For example, salt exchange reaction between the acid derived from anion (i.e., a compound in which —COO— becomes —COOH when A⁻ is —COO—, a compound in which —SO₂O— becomes —SO₂OH when A⁻ is —SO₂O—) in the compound represented by the formula (c1) and a salt having Mⁿ⁺, the compound represented by the formula (c1) can be obtained.

The content of the compound represented by the formula (c1) is not particularly limited, but the compound represented by the formula (c1) is used preferably in the content in a range of 0.01 parts by mass or more and 5 parts by mass or less, more preferably 0.10 parts by mass or more and 3 parts by mass or less, and particularly preferably 0.25 parts by mass or more and 1 parts by mass or less with respect to the total mass of 100 parts by mass of the above resin (B) and the following alkali-soluble resin (D).

### <Acid Diffusion Control Agent (C′)>

The photosensitive resin composition may contain an acid diffusion control agent (C′) other than the compound represented by the formula (c1). The acid diffusion control agent (C′) other than the compound represented by the formula (c1) is preferably a nitrogen-containing compound (C′1), and an organic carboxylic acid, or an oxo acid of phosphorus or a derivative thereof (C′2) may be further contained as needed.

### [Nitrogen-Containing Compound (C′1)]

Examples of the nitrogen-containing compound (C′1) include trimethylamine, diethylamine, triethylamine, di-n-propylamine, tri-n-propylamine, tri-n-pentylamine, tribenzylamine, diethanolamine, triethanolamine, n-hexylamine, n-heptyl amine, n-octyl amine, n-nonyl amine, ethylenediamine, N,N,N′,N′-tetramethylethylenediamine, tetramethylenediamine, hexamethylenediamine, 4,4′-diaminodiphenylmethane, 4,4′-diaminodiphenyl ether, 4,4′-diaminobenzophenone, 4,4′-diaminodiphenylamine, formamide, N-methylformamide, N,N-dimethylformamide, acetamide, N-methylacetamide, N,N-dimethylacetamide, propionamide, benzamide, pyrrolidone, N-methylpyrrolidone, methylurea, 1,1-dimethylurea, 1,3-dimethylurea, 1,1,3,3,-tetramethylurea, 1,3-diphenylurea, imidazole, benzimidazole, 4-methylimidazole, 8-oxyquinoline, acridine, purine, pyrrolidine, piperidine, 2,4,6-tri(2-pyridyl)-S-triazine, morpholine, 4-methylmorpholine, piperazine, 1,4-dimethylpiperazine, 1,4-diazabicyclo[2.2.2]octane, and pyridine. These may be used alone, or in combinations of two or more thereof.

Furthermore, commercially available hindered amine compounds such as Adeka Stab LA-52, Adeka Stab LA-57, Adeka Stab LA-63P, Adeka Stab LA-68, Adeka Stab LA-72, Adeka Stab LA-77Y, Adeka Stab LA-77G, Adeka Stab LA-81, Adeka Stab LA-82, Adeka Stab LA-87 (all manufactured by ADEKA), and 4-hydroxy-1,2,2,6,6-pentamethyl pyperidine derivatives, and the like, and pyridine having a 2,6-position has been substituted with a substituent a hydrocarbon group, such as 2,6-diphenyl pyridine and 2,6-di-tert-butyl pyridine, can be used as the nitrogen-containing compound (C′1).

The nitrogen-containing compound (C′1) may be used in an amount usually in the range of 0 parts by mass or more and 5 parts by mass or less, and particularly preferably in the range of 0 parts by mass or more and 3 parts by mass or less, with respect to 100 parts by mass of total mass of the above resin (B) and the following alkali-soluble resin (D).

### [Organic Carboxylic Acid or Oxo Acid of Phosphorus or Derivative Thereof (C′2)]

Among the organic carboxylic acid, or the oxo acid of phosphorus or the derivative thereof (C′2), specific preferred examples of the organic carboxylic acid include malonic acid, citric acid, malic acid, succinic acid, benzoic acid, salicylic acid and the like, and salicylic acid is particularly preferred.

Examples of the oxo acid of phosphorus or derivatives thereof include phosphoric acid and derivatives such as esters thereof such as phosphoric acid, phosphoric acid di-n-butyl ester, and phosphoric acid diphenyl ester; phosphonic acid and derivatives such as esters thereof such as phosphonic acid, phosphonic acid dimethyl ester, phosphonic acid di-n-butyl ester, phenylphosphonic acid, phosphonic acid diphenyl ester, and phosphonic acid dibenzyl ester; and phosphinic acid and derivatives such as esters thereof such as phosphinic acid and phenylphosphinic acid; and the like. Among these, phosphonic acid is particularly preferable. These may be used alone, or in combinations of two or more thereof.

The organic carboxylic acid or oxo acid of phosphorus or derivative thereof (C′2) may be used in an amount usually in the range of 0 parts by mass or more and 5 parts by mass or less, and particularly preferably in the range of 0 parts by mass and 3 parts by mass or less, with respect to 100 parts by mass of total mass of the above resin (B) and the following alkali-soluble resin (D).

Moreover, in order to form a salt to allow for stabilization, the organic carboxylic acid, or the oxo acid of phosphorous or the derivative thereof (C′2) is preferably used in an amount equivalent to that of the above nitrogen-containing compound (C′1).

### <Alkali-Soluble Resin (D)>

It is preferable that the photosensitive resin composition further contains an alkali-soluble resin (D) in order to improve alkali-solubility. Here, the alkali-soluble resin refers to a resin in which a resin solution having a resin concentration of 20% by mass (solvent: propylene glycol monomethyl ether acetate) is used to form a resin film having a thickness of 1 μm on a substrate and when the resin film is immersed in a TMAM (tetramethylammonium hydroxide) aqueous solution of 2.38% by mass for 1 minute, the resin film of 0.01 μm or more is dissolved, and does not correspond to the above-mentioned (B) component (typically, resin having alkali-solubility that does not substantially changed due to the action of acid). The alkali-soluble resin (D) is preferably at least one resin selected from the group consisting of novolak resin (D1), polyhydroxystyrene resin (D2), and acrylic resin (D3).

### [Novolak Resin (D1)]

The novolak resin may be obtained by addition fusion between, for example, aromatic compounds having a phenolic hydroxyl group (hereinafter, merely referred to as “phenols”) and aldehydes in the presence of an acid catalyst.

Examples of the phenols include, for example, phenol, o-cresol, m-cresol, p-cresol, o-ethylphenol, m-ethylphenol, p-ethylphenol, o-butylphenol, m-butylphenol, p-butylphenol, 2,3-xylenol, 2,4-xylenol, 2,5-xylenol, 2,6-xylenol, 3,4-xylenol, 3,5-xylenol, 2,3,5-trimethyl phenol, 3,4,5-trimethyl phenol, p-phenylphenol, resorcinol, hydroquinone, hydroquinone monomethyl ether, pyrogallol, phloroglycinol, hydroxydiphenyl, bisphenol A, gallic acid, gallic acid ester, α-naphthol, β-naphthol, and the like. Examples of the aldehydes include formaldehyde, furfural, benzaldehyde, nitrobenzaldehyde, acetaldehyde, and the like. The catalyst used in the addition fusion reaction, which is not specifically limited, is exemplified by hydrochloric acid, nitric acid, sulfuric acid, formic acid, oxalic acid, acetic acid, etc., for acid catalyst.

The flexibility of the novolak resins can be enhanced still more when o-cresol is used, a hydrogen atom of a hydroxide group in the resins is substituted with other substituents, or bulky aldehydes are used.

The mass average molecular weight of novolac resin (D1) is not particularly limited as long as the object of the present invention is not impaired, but the mass average molecular weight is preferably 1000 or more and 50000 or less.

### [Polyhydroxystyrene Resin (D2)]

Examples of the hydroxystyrene compound constituting the polyhydroxystyrene resin (D2) include p-hydroxystyrene, α-methylhydroxystyrene, α-ethylhydroxystyrene, and the like. Furthermore, the polyhydroxystyrene resin (D2) is preferably a copolymer with a styrene resin. Examples of the styrene compound to constitute such a styrene resin include styrene, chlorostyrene, chloromethylstyrene, vinyltoluene, α-methylstyrene, and the like.

The mass average molecular weight of the polyhydroxystyrene resin (D2) is not particularly limited as long as the object of the present invention is not impaired, but the mass average molecular weight is preferably 1000 or more and 50000 or less.

### [Acrylic Resin (D3)]

It is preferable that the acrylic resin (D3) includes a constituent unit derived from a polymerizable compound having an ether bond and a constituent unit derived from a polymerizable compound having a carboxyl group.

Examples of the above polymerizable compound having an ether bond include (meth)acrylic acid derivatives having an ether bond and an ester bond such as 2-methoxyethyl (meth)acrylate, methoxytriethylene glycol (meth)acrylate, 3-methoxybutyl (meth) acrylate, ethylcarbitol (meth) acrylate, phenoxypolyethylene glycol (meth) acrylate, methoxypolypropylene glycol (meth) acrylate, tetrahydrofurfuryl (meth)acrylate, and the like. The above polymerizable compound having an ether bond is preferably, 2-methoxyethyl acrylate, and methoxytriethylene glycol acrylate. These polymerizable compounds may be used alone, or in combinations of two or more.

Examples of the above polymerizable compound having a carboxy group include monocarboxylic acids such as acrylic acid, methacrylic acid and crotonic acid; dicarboxylic acids such as maleic acid, fumaric acid and itaconic acid; compounds having a carboxy group and an ester bond such as 2-methacryloyloxyethyl succinic acid, 2-methacryloyloxyethyl maleic acid, 2-methacryloyloxyethyl phthalic acid, 2-methacryloyloxyethyl hexahydrophthalic acid and the like. The above polymerizable compound having a carboxy group is preferably, acrylic acid and methacrylic acid. These polymerizable compounds may be used alone, or in combinations of two or more thereof.

The mass average molecular weight of the acrylic resin (D3) is not particularly limited as long as the object of the present invention is not impaired, but the mass average molecular weight is preferably 50000 or more and 800000 or less.

The content of the alkali-soluble resin (D) is preferably 3 parts by mass or more and 80 parts by mass or less, and more preferably 5 parts by mass or more and 70 parts by mass or less when the total solid content of the photosensitive resin composition is 100 parts by mass. When the content of the alkali-soluble resin (D) is in the above-mentioned range, the alkali-solubility is easily improved.

### <Sulfur-Containing Compound (E)>

When a photosensitive resin composition is used for pattern formation on a metal substrate, the photosensitive resin composition preferably includes a sulfur-containing compound (E). The sulfur-containing compound (E) is a compound including a sulfur atom that can coordinate with metal. Note here that in a compound that can generate two or more tautomers, when at least one tautomer includes a sulfur atom that coordinates with metal constituting a metal substrate surface, the compound corresponds to a sulfur-containing compound. When a resist pattern serving as a template for plating is formed on a surface made of metal such as Cu, defectives such as footing having a cross-sectional shape easily occur. However, when the photosensitive resin composition includes a sulfur-containing compound (E), even when a resist pattern is formed on a surface made of metal in a substrate, defectives such as footing having a cross-sectional shape is easily suppressed. Note here that the “footing” is a phenomenon in which the width of the bottom becomes narrower than that of the top in a nonresist portion due to protrusion of the resist portion toward the nonresist portion in the vicinity of the contacting surface between the substrate surface and the resist pattern. When the photosensitive resin composition is used for pattern formation on a substrate other than the metal substrate, the photosensitive resin composition does not specially need to include a sulfur-containing compound. When the photosensitive resin composition is used for pattern formation on the substrate other than the metal substrate, it is preferable that the photosensitive resin composition does not include a sulfur-containing compound (E) from the viewpoint that reduction of the number of components in the photosensitive resin composition makes the production of the photosensitive resin composition easier, and reduces the production cost of the photosensitive resin composition, and the like. Note here that there is no particular deficiency resulting from the inclusion of a sulfur-containing compound (E) in the photosensitive resin composition to be used for formation of pattern on the substrate other than the metal substrate.

The sulfur atom that can coordinate with metal is included in a sulfur-containing compound as, for example, a mercapto group (—SH), a thiocarboxy group (—CO—SH), a dithiocarboxy group (—CS—SH), a thiocarbonyl group (—CS—), and the like. From the viewpoint of easiness in coordinating with metal and being excellent in suppressing footing, the sulfur-containing compound preferably includes a mercapto group.

Preferable examples of the sulfur-containing compound having a mercapto group include compounds represented by the following formula (e1).

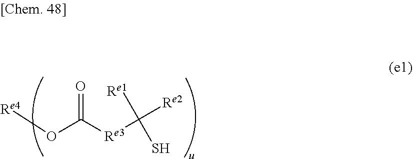

(In the formula, R^{e1} and R^{e2} each independently represents a hydrogen atom or an alkyl group, R^{e3} represents a single bond or an alkylene group, R^{e4} represents a u-valence aliphatic group which may include an atom other than carbon, and u is an integer of 2 or more and 4 or less.)

R^{e1} and R^{e2} are an alkyl group, the alkyl group may be linear or branched, and is preferably linear. When R^{e1} and R^{e2} are an alkyl group, the number of carbon atoms of the alkyl group is not particularly limited within a range in which the objects of the present invention are not impaired. The number of carbon atoms of the alkyl group is preferably 1 or more and 4 or less, particularly preferably 1 or 2, and the most preferably 1. As the combination of R^{e1} and R^{e2}, preferably, one is a hydrogen atom and the other is an alkyl group, and particularly preferably one is a hydrogen atom and the other is a methyl group.

When R^{e3} is an alkylene group, the alkylene group may be linear or branched, and is preferably linear. When R^{e3} is an alkylene group, the number of carbon atoms of the alkylene group is not particularly limited within a range in which the objects of the present invention are not impaired. The number of carbon atoms of the alkylene group is preferably 1 or more and 10 or less, more preferably 1 or more and 5 or less, particularly preferably 1 or 2, and the most preferably 1.

R^{e4} is an aliphatic group having two or more and four or less valences and which may include an atom other than carbon atom. Examples of the atoms which may be included in R^{e4} include a nitrogen atom, an oxygen atom, a sulfur atom, a fluorine atom, a chlorine atom, a bromine atom, an iodine atom, and the like. A structure of the aliphatic group as R^{e4} may be linear or branched, or may be cyclic, and a structure combining these structures.

Among the compounds represented by the formula (e1), a compound represented by the following formula (e2) is more preferable.

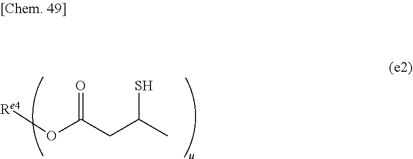

(In the formula (e2), R^{e4} and u are the same as those in the formula (e1).)

Among the compounds represented by the above formula (e2), the following compounds are preferable.

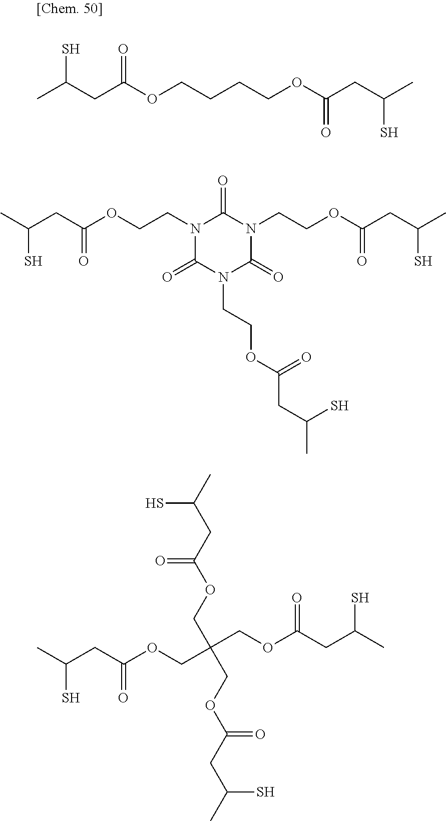

Compounds represented by the following formulae (e3-L1) to (e3-L7) are also preferable examples of the sulfur-containing compound having a mercapto group.

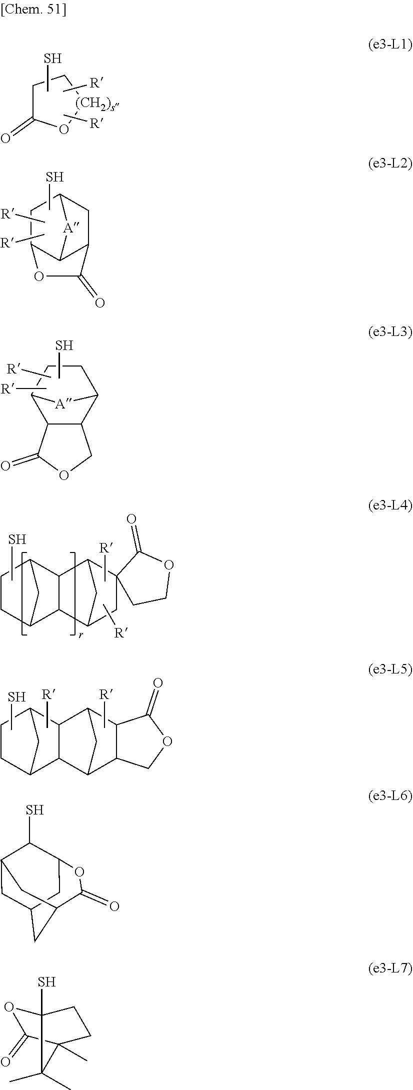

(In the formulae (e3-L1) to (e3-L7), R′, s″, A″, and r are the same as in the formulae (b-L1) to (b-L7) described for the acrylic resin (B3).)

Suitable specific examples of the mercapto compound represented by the above formulae (e3-L1) to (e3-L7) include the following compounds.

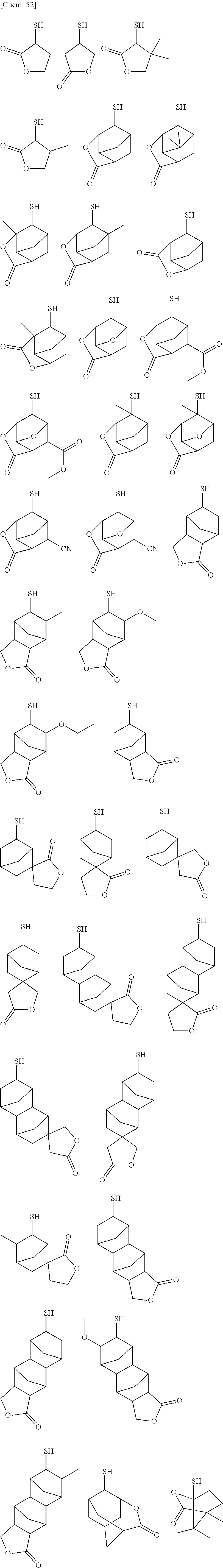

Compounds represented by the following formulae (e3-1) to (e3-4) are also preferable examples of the sulfur-containing compound having a mercapto group.

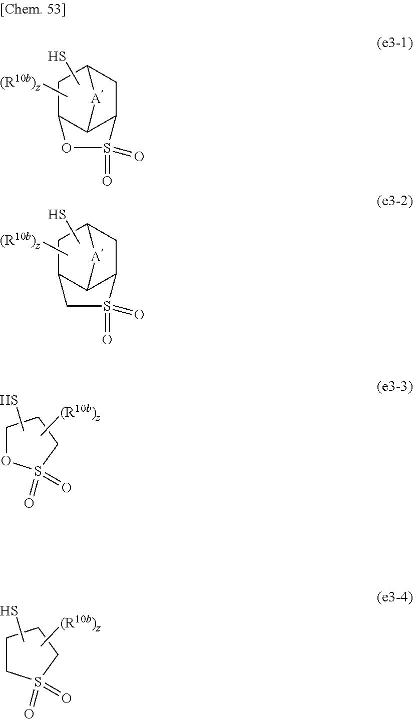

(In the formulae (e3-1) to (e3-4), definitions of abbreviations are the same as mentioned for the formulae (3-1) to (3-4) described for acrylic resin (B3).)

Suitable specific examples of the mercapto compound represented by the above formulae (e3-1) to (e3-4) include the following compounds.

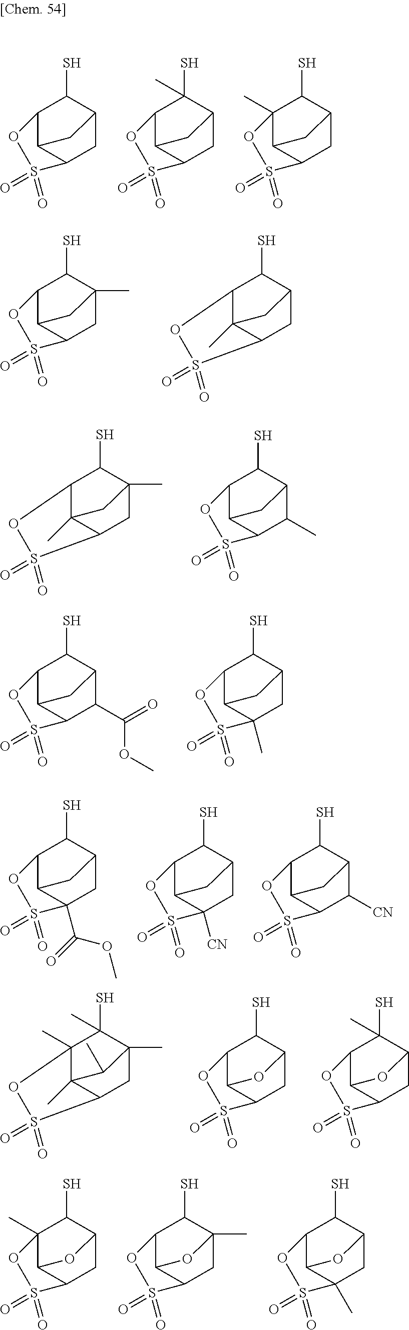

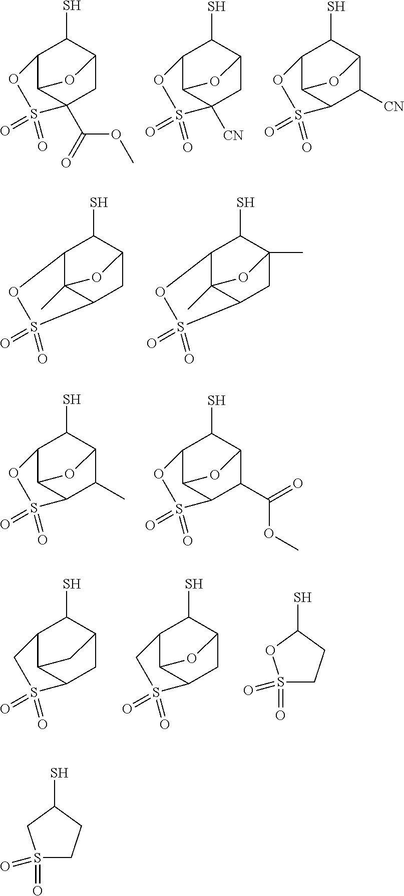

Furthermore, preferable examples of the compound having a mercapto group include compounds represented by the following formula (e4).

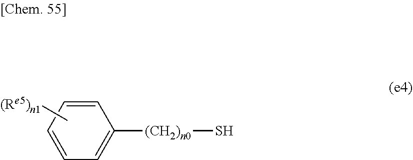

(In the formula (e4), R^{e5} is a group selected from the group consisting of a hydroxyl group, an alkyl group having 1 or more 4 or less carbon atoms, an alkoxy group having 1 or more 4 or less carbon atoms, an alkylthio group having 1 or more and 4 or less carbon atoms, a hydroxyalkyl group having 1 or more and 4 or less carbon atoms, a mercapto alkyl group having 1 or more and 4 or less carbon atoms, a halogenated alkyl group having 1 or more and 4 or less carbon atoms, and a halogen atom, n1 is an integer of 0 or more and 3 or less, n0 is an integer of 0 or more and 3 or less, when n1 is 2 or 3, R^{e5} may be the same as or different from each other.)

Specific examples of the case where Res is an alkyl group which may have a hydroxyl group having 1 or more 4 or less carbon atoms include a methyl group, an ethyl group, an n-propyl group, an isopropyl group, an n-butyl group, an isobutyl group, a sec-butyl group, and a tert-butyl group. Among these alkyl groups, a methyl group, a hydroxymethyl group, and an ethyl group are preferable.

Specific examples of the case where Res is an alkoxy group having 1 or more 4 or less carbon atoms include a methoxy group, an ethoxy group, an n-propyloxy group, an isopropyloxy group, an n-butyloxy group, an isobutyloxy group, a sec-butyloxy group, and a tert-butyloxy group. Among these alkoxy groups, a methoxy group and an ethoxy group are preferable, and a methoxy group is more preferable.

Specific examples of the case where Res is an alkylthio group having 1 or more 4 or less carbon atoms include a methylthio group, an ethylthio group, an n-propylthio group, an isopropylthio group, an n-butylthio group, an isobutylthio group, a sec-butylthio group, and a tert-butylthio group. Among these alkylthio groups, a methylthio group, and an ethylthio group are preferable, and a methylthio group is more preferable.

Specific examples of the case where Res is a hydroxyalkyl group having 1 or more 4 or less carbon atoms include a hydroxymethyl group, a 2-hydroxyethyl group, a 1-hydroxyethyl group, a 3-hydroxy-n-propyl group, and a 4-hydroxy-n-butyl group, and the like. Among these hydroxyalkyl groups, a hydroxymethyl group, a 2-hydroxyethyl group, and a 1-hydroxyethyl group are preferable, and a hydroxymethyl group is more preferable.

Specific examples of the case where Res is a mercapto alkyl group having 1 or more 4 or less carbon atoms include a mercapto methyl group, a 2-mercapto ethyl group, a 1-mercapto ethyl group, a 3-mercapto-n-propyl group, a 4-mercapto-n-butyl group, and the like. Among these mercapto alkyl groups, a mercapto methyl group, a 2-mercapto ethyl group, and 1-mercapto ethyl group are preferable, and a mercapto methyl group is more preferable.

When R^{e5} is a halogenated alkyl group having 1 or more 4 or less carbon atoms, examples of the halogen atom included in the halogenated alkyl group include fluorine, chlorine, bromine, iodine, and the like. Specific examples of the case where R^{e5} is a halogenated alkyl group having 1 or more 4 or less carbon atoms include a chloromethyl group, a bromomethyl group, an iodomethyl group, a fluoromethyl group, a dichloromethyl group, a dibromomethyl group, a difluoromethyl group, a trichloromethyl group, a tribromomethyl group, a trifluoromethyl group, a 2-chloroethyl group, a 2-bromoethyl group, a 2-fluoroethyl group, a 1,2-dichloroethyl group, a 2,2-difluoroethyl group, a 1-chloro-2-fluoroethyl group, 3-chloro-n-propyl group, a 3-bromo-n-propyl group, a 3-fluoro-n-propyl group, 4-chloro-n-butyl group, and the like. Among these halogenated alkyl groups, a chloromethyl group, a bromomethyl group, an iodomethyl group, a fluoromethyl group, a dichloromethyl group, a dibromomethyl group, a difluoromethyl group, a trichloromethyl group, a tribromomethyl group, and a trifluoromethyl group are preferable, and a chloromethyl group, a dichloromethyl group, a trichloromethyl group, and a trifluoromethyl group are more preferable.

Specific examples of the case where Res is a halogen atom include fluorine, chlorine, bromine, or iodine.

In the formula (e4), n1 is an integer of 0 or more 3 or less, and 1 is more preferable. When n1 is 2 or 3, a plurality of R^{e5}s may be the same as or different from each other.

In the compound represented by the formula (e4), a substituted position of Res on a benzene ring is not particularly limited. The substituted position of Res on a benzene ring is preferably a meta position or a para position with respect to the bond position of —(CH₂)ₙ₀—SH.

The compound represented by the formula (e4) is preferably a compound having at least one group selected from the group consisting of an alkyl group, a hydroxyalkyl group, and a mercapto alkyl group as R^{e5}, and more preferably a compound having one group selected from the group consisting of an alkyl group, a hydroxyalkyl group, and a mercapto alkyl group as R^{e5}. When the compound represented by the formula (e4) has one group selected from the group consisting of an alkyl group, a hydroxyalkyl group, and a mercapto alkyl group as Res, the substituted position on the benzene ring of the alkyl group, the hydroxyalkyl group, or the mercapto alkyl group is preferably a meta position or a para position with respect to the bond position of —(CH₂)ₙ₀—SH, and more preferably a para position.

In the formula (e4), n0 is an integer of 0 or more 3 or less. From the viewpoint that preparation or availability of a compound is easy, n0 is preferably 0 or 1, and more preferably 0.

Specific examples of the compound represented by the formula (e4) include p-mercaptophenol, p-thiocresol, m-thiocresol, 4-(methylthio)benzenethiol, 4-methoxybenzenethiol, 3-methoxybenzenethiol, 4-ethoxybenzenethiol, 4-isopropyloxy benzenethiol, 4-tert-butoxybenzenethiol, 3,4-dimethoxy benzenethiol, 3,4,5-trimethoxybenzenethiol, 4-ethylbenzenethiol, 4-isopropyl benzenethiol, 4-n-butylbenzenethiol, 4-tert-butylbenzenethiol, 3-ethylbenzenethiol, 3-isopropyl benzenethiol, 3-n-butylbenzenethiol, 3-tert-butylbenzenethiol, 3,5-dimethyl benzenethiol, 3,4-dimethyl benzenethiol, 3-tert-butyl-4-methylbenzenethiol, 3-tert-4-methylbenzenethiol, 3-tert-butyl-5-methylbenzenethiol, 4-tert-butyl-3-methylbenzenethiol, 4-mercaptobenzyl alcohol, 3-mercaptobenzyl alcohol, 4-(mercaptomethyl)phenol, 3-(mercaptomethyl)phenol, 1,4-di(mercaptomethyl)phenol, 1,3-di(mercaptomethyl)phenol, 4-fluorobenzenethiol, 3-fluorobenzenethiol, 4-chlorobenzenethiol, 3-chlorobenzenethiol, 4-bromobenzenethiol, 4-iodobenzenethiol, 3-bromobenzenethiol, 3,4-dichlorobenzenethiol, 3,5-dichlorobenzenethiol, 3,4-difluorobenzenethiol, 3,5-difluorobenzenethiol, 4-mercaptocatechol, 2,6-di-tert-butyl-4-mercaptophenol, 3,5-di-tert-butyl-4-methoxybenzenethiol, 4-bromo-3-methylbenzenethiol, 4-(trifluoromethyl)benzenethiol, 3-(trifluoromethyl)benzenethiol, 3,5-bis(trifluoromethyl)benzenethiol, 4-methylthiobenzenethiol, 4-ethylthiobenzenethiol, 4-n-butylthiobenzenethiol, and 4-tert-butylthiobenzenethiol, and the like.

Furthermore, examples of the sulfur-containing compound having a mercapto group include a compound including nitrogen-containing aromatic heterocycle substituted with a mercapto group, and a tautomer of a compound including nitrogen-containing aromatic heterocycle substituted with a mercapto group. Preferable specific examples of the nitrogen-containing aromatic heterocycle include imidazole, pyrazole, 1,2,3-triazol, 1,2,4-triazol, oxazole, thiazole, pyridine, pyrimidine, pyridazine, pyrazine, 1,2,3-triazine, 1,2,4-triazine, 1,3,5-triazine, indole, indazole, benzimidazole, benzoxazole, benzothiazole, 1H-benzotriazole, quinoline, isoquinoline, cinnoline, phthalazine, quinazoline, quinoxaline, and 1,8-naphthyridine.

Suitable specific examples of a nitrogen-containing heterocyclic compound suitable as a sulfur-containing compound, and a tautomer of the nitrogen-containing heterocyclic compound include the following compounds.

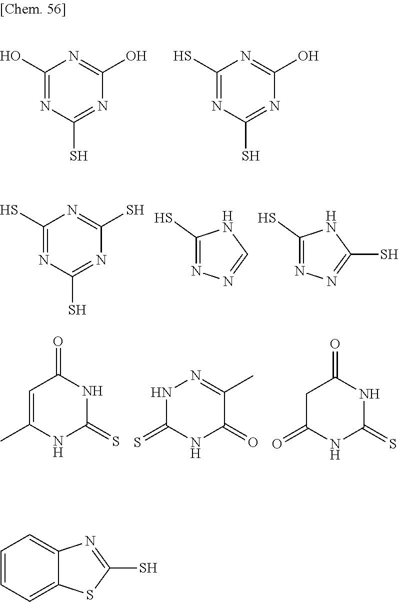

When the photosensitive resin composition includes a sulfur-containing compound (E), the use amount is preferably 0.01 parts by mass or more 5 parts by mass or less, more preferably 0.02 parts by mass or more 3 parts by mass or less, and particularly preferably 0.05 parts by mass or more 2 parts by mass or less with respect to 100 parts by mass that is the total mass of the above resin (B) and the alkali-soluble resin (D).

### <Organic Solvent (S)>

It is preferable that the photosensitive resin composition contains an organic solvent (S). The types of the organic solvent (S) are not particularly limited within a range in which the objects of the present invention are not impaired, and an organic solvent appropriately selected from organic solvents conventionally used for positive-type photosensitive resin compositions can be used.

Specific examples of the organic solvent (S) include ketones such as acetone, methyl ethyl ketone, cyclohexanone, methyl isoamyl ketone, and 2-heptanone; polyhydric alcohols and derivatives thereof such as a monomethyl ether, a monoethyl ether, a monopropyl ether, a monobutyl ether, and a monophenyl ether of ethylene glycol, ethylene glycol monoacetate, diethylene glycol, diethylene glycol monoacetate, propylene glycol, propylene glycol monoacetate, propylene glycol monomethyl ether acetate, dipropylene glycol, and dipropylene glycol monoacetate; cyclic ethers such as dioxane; esters such as ethyl formate, methyl lactate, ethyl lactate, methyl acetate, ethyl acetate, butyl acetate, methyl pyruvate, methyl acetoacetate, ethyl acetoacetate, ethyl pyruvate, ethylethoxy acetate, methyl methoxypropionate, ethyl ethoxypropionate, methyl 2-hydroxypropionate, ethyl 2-hydroxypropionate, ethyl 2-hydroxy-2-methylpropionate, methyl 2-hydroxy-3-methylbutanate, 3-methoxybutyl acetate, and 3-methyl-3-methoxybutyl acetate; aromatic hydrocarbons such as toluene and xylene; and the like. These may be used alone, or as a mixture of two or more thereof.

The content of the organic solvent (S) is not particularly limited within a range where the objects of the present invention are not impaired. In a case where a photosensitive resin composition is used for a thick-film application such that a photosensitive layer obtained by the spin coating method and the like has a film thickness of 5 μm or more, the organic solvent (S) is preferably used in a range where the solid content concentration of the photosensitive resin composition is 30% by mass or more and 70% by mass or less.

### <Other Components>

The photosensitive resin composition may further contain a polyvinyl resin for improving plasticity. Specific examples of the polyvinyl resin include polyvinyl chloride, polystyrene, polyhydroxystyrene, polyvinyl acetate, polyvinylbenzoic acid, polyvinyl methyl ether, polyvinyl ethyl ether, polyvinyl alcohol, polyvinyl pyrrolidone, polyvinyl phenol, and copolymers thereof, and the like. The polyvinyl resin is preferably polyvinyl methyl ether in view of low glass transition temperatures.

Furthermore, the photosensitive resin composition may also contain an adhesive auxiliary agent in order to improve the adhesiveness between a template and the like formed with the photosensitive resin composition and a substrate.

Also, the photosensitive resin composition may further contain a surfactant for improving coating characteristics, defoaming characteristics, leveling characteristics, and the like. As the surfactant, for example, a fluorine-based surfactant or a silicone-based surfactant is preferably used. Specific examples of the fluorine-based surfactant include commercially available fluorine-based surfactants such as BM-1000 and BM-1100 (both manufactured by B.M-Chemie Co., Ltd.), Megafac F142D, Megafac F172, Megafac F173 and Megafac F183 (all manufactured by Dainippon Ink And Chemicals, Incorporated), Flolade FC-135, Flolade FC-170C, Flolade FC-430, and Flolade FC-431 (all manufactured by Sumitomo 3M Ltd.), Surflon S-112, Surflon S-113, Surflon S-131, Surflon S-141, and Surflon S-145 (all manufactured by Asahi Glass Co., Ltd.), SH-28PA, SH-190, SH-193, SZ-6032, and SF-8428 (all manufactured by Toray Silicone Co., Ltd.) and the like, but not limited thereto. As the silicone-based surfactant, an unmodified silicone-based surfactant, a polyether modified silicone-based surfactant, a polyester modified silicone-based surfactant, an alkyl modified silicone-based surfactant, an aralkyl modified silicone-based surfactant, a reactive silicone-based surfactant, and the like, can be preferably used. As the silicone-based surfactant, commercially available silicone-based surfactant can be used. Specific examples of the commercially available silicone-based surfactant include Paintad M (manufactured by Dow Corning Toray Co., Ltd.), Topica K1000, Topica K2000, and Topica K5000 (all manufactured by Takachiho Sangyo Co., Ltd.), XL-121 (polyether modified silicone-based surfactant, manufactured by Clariant Co.), BYK-310 (polyester modified silicone-based surfactant, manufactured by BYK), and the like.

Furthermore, in order to finely adjust the solubility in a developing solution, the photosensitive resin composition may further contain an acid, an acid anhydride, or a solvent having a high boiling point.

Specific examples of the acid and acid anhydride include monocarboxylic acids such as acetic acid, propionic acid, n-butyric acid, isobutyric acid, n-valeric acid, isovaleric acid, benzoic acid, and cinnamic acid; hydroxymonocarboxylic acids such as lactic acid, 2-hydroxybutyric acid, 3-hydroxybutyric acid, salicylic acid, m-hydroxybenzoic acid, p-hydroxybenzoic acid, 2-hydroxycinnamic acid, 3-hydroxycinnamic acid, 4-hydroxycinnamic acid, 5-hydroxyisophthalic acid, and syringic acid; polyvalent carboxylic acids such as oxalic acid, succinic acid, glutaric acid, adipic acid, maleic acid, itaconic acid, hexahydrophthalic acid, phthalic acid, isophthalic acid, terephthalic acid, 1,2-cyclohexanedicarboxylic acid, 1,2,4-cyclohexanetricarboxylic acid, butanetetracarboxylic acid, trimellitic acid, pyromellitic acid, cyclopentanetetracarboxylic acid, butanetetracarboxylic acid, and 1,2,5,8-naphthalenetetracarboxylic acid; acid anhydrides such as itaconic anhydride, succinic anhydride, citraconic anhydride, dodecenylsuccinic anhydride, tricarbanilic anhydride, maleic anhydride, hexahydrophthalic anhydride, methyltetrahydrophthalic anhydride, Himic anhydride, 1,2,3,4-butanetetracarboxylic anhydride, cyclopentanetetracarboxylic dianhydride, phthalic anhydride, pyromellitic anhydride, trimellitic anhydride, benzophenonetetracarboxylic anhydride, ethylene glycol bis anhydrous trimellitate, and glycerin tris anhydrous trimellitate; and the like.

Furthermore, specific examples of the solvent having a high boiling point include N-methylformamide, N,N-dimethylformamide, N-methylformanilide, N-methylacetamide, N,N-dimethlyacetamide, N-methylpyrrolidone, dimethyl sulfoxide, benzyl ethyl ether, dihexyl ether, acetonyl acetone, isophorone, caproic acid, caprylic acid, 1-octanol, 1-nonanol, benzyl alcohol, benzyl acetate, ethyl benzoate, diethyl oxalate, diethyl maleate, γ-butyrolactone, ethylene carbonate, propylene carbonate, phenyl cellosolve acetate, and the like.

Moreover, the photosensitive resin composition may further contain a sensitizer for improving the sensitivity.

### <Method for Preparing Chemically Amplified Positive-Type Photosensitive Resin Composition>

A chemically amplified positive-type photosensitive resin composition is prepared by mixing and stirring the each of the above components by the common method. Devices capable of being used for mixing and stirring the above components include dissolvers, homogenizers, 3-roll mills, and the like. After uniformly mixing the above components, the resulting mixture may be filtered through a mesh, a membrane filter, and the like.

### <<Photosensitive Dry Film>>

A photosensitive dry film includes a substrate film, and a photosensitive layer formed on the surface of the substrate film. The photosensitive layer is made of the aforementioned photosensitive resin compositions.

As the substrate film, a film having optical transparency is preferable. Specific examples thereof include a polyethylene terephthalate (PET) film, a polypropylene (PP) film, a polyethylene (PE) film, and the like. In view of excellent balance between the optical transparency and the breaking strength, a polyethylene terephthalate (PET) film is preferable.

The aforementioned photosensitive resin composition is applied on the substrate film to form a photosensitive layer, and thereby a photosensitive dry film is produced. When the photosensitive layer is formed on the substrate film, a photosensitive resin composition is applied and dried on the substrate film using an applicator, a bar coater, a wire bar coater, a roller coater, a curtain flow coater, and the like, so that a film thickness after drying is preferably 0.5 μm or more and 300 μm or less, more preferably 1 μm or more and 300 μm or less, and particularly preferably 3 μm or more and 100 μm or less.

The photosensitive dry film may include a protective film on the photosensitive layer. Examples of the protective film include a polyethylene terephthalate (PET) film, a polypropylene (PP) film, a polyethylene (PE) film, and the like.

<<Method for Producing Patterned Resist Film, and Substrate with Template>>

A method for forming a patterned resist film on a substrate using the photosensitive resin composition described above is not particularly limited. Such a patterned resist film is suitably used as a template for forming a plated article, an etching mask when a substrate is processed by etching. A suitable method includes a producing method of a patterned resist film, the method including:

a laminating step laminating a photosensitive layer on a substrate, the layer being formed from a photosensitive resin composition;
a exposure step of exposing the photosensitive layer through irradiation with an active ray or radiation in a position-selective manner; and
a developing step of developing the exposed photosensitive layer. A method for producing a substrate provided with a template, having a template for forming a plated article, is the same method as the method for producing a patterned resist film except that the method includes a step of laminating a photosensitive layer on a metal surface of the substrate having a metal surface, and a template for forming a plated article is produced by developing in the developing step.

The substrate on which the photosensitive layer is laminated is not particularly limited, and conventionally known substrates can be used. Examples thereof include substrates for electronic components, and the substrate on which a predetermined wiring pattern is formed. As the substrate, a silicon substrate, glass substrate, or the like, can be used. When a substrate provided with a template, having a template for forming a plated article, is manufactured, for the substrate, a substrate having a metal surface is used. As metal species constituting the metal surface, copper, gold, and aluminum are preferable, and copper is more preferable.

The photosensitive layer is laminated on the substrate, for example, as follows. In other words, a liquid photosensitive resin composition is applied to a substrate, followed by removing the solvent by heating to form a photosensitive layer having a desired thickness. The thickness of the photosensitive layer is not particularly limited as long as it is possible to form a resist pattern as a template having a desired thickness. The thickness of the photosensitive layer is not particularly limited, but is preferably 0.5 μm or more, more preferably 0.5 μm or more and 300 μm or less, further preferably 1 μm or more and 150 μm or less, and particularly preferably 3 μm or more and 100 μm or less.

As a method for applying a photosensitive resin composition onto a substrate, methods such as the spin coating method, the slit coating method, the roll coating method, the screen printing method, and the applicator method can be employed. It is preferable that the photosensitive layer is subjected to pre-baking. The conditions of the pre-baking may differ depending on the components in the photosensitive resin composition, the blending ratio, the thickness of a coating film, and the like, but usually about 2 minutes or more and 120 minutes or less at 70° C. or more and 200° C. or less, and preferably 80° C. or more and 150° C. or less.

The photosensitive layer formed as described above is selectively irradiated (exposed) with an active ray or radiation, for example, an ultraviolet radiation or visible light with a wavelength of 300 nm or more and 500 nm or less, for example, a g-ray (wavelength: 436 nm), an h-ray (wavelength: 405 nm), and an i-ray (wavelength: 365 nm) through a mask having a predetermined pattern.

Low pressure mercury lamps, high pressure mercury lamps, super-high pressure mercury lamps, metal halide lamps, argon gas lasers, and the like, can be used for the light source of the radiation. The radiation may include micro waves, infrared rays, visible lights, ultraviolet rays, X-rays, γ-rays, electron beams, proton beams, neutron beams, ion beams, and the like. The irradiation dose of the radiation may vary depending on the constituent of the photosensitive resin composition, the film thickness of the photosensitive layer, and the like. For example, when a super-high pressure mercury lamp is used, the dose may be 100 mJ/cm² or more and 10000 mJ/cm² or less. The radiation includes a light ray to activate the acid generating agent (A) in order to generate an acid.

After the exposure, the diffusion of acid is promoted by heating the photosensitive layer using a well-known method to change the alkali solubility of the photosensitive layer at an exposed portion in the photosensitive resin film.

Subsequently, the exposed photosensitive layer is developed according to a conventionally known method, and an unnecessary portion is dissolved and removed to form a predetermined resist pattern or a template for forming plated articles. At this time, as the developing solution, an alkaline aqueous solution is used.

As the developing solution, an aqueous solution of an alkali such as, for example, sodium hydroxide, potassium hydroxide, sodium carbonate, sodium silicate, sodium metasilicate, aqueous ammonia, ethylamine, n-propylamine, diethylamine, di-n-propylamine, triethylamine, methyldiethylamine, dimethylethanolamine, triethanolamine, tetramethylammonium hydroxide (tetramethylammonium hydroxide), tetraethylammonium hydroxide, pyrrole, piperidine, 1,8-diazabicyclo[5.4.0]-7-undecene, or 1,5-diazabicyclo[4.3.0]—S— nonane can be used. Also, an aqueous solution prepared by adding an adequate amount of a water-soluble organic solvent such as methanol or ethanol, or a surfactant to the above aqueous solution of the alkali can be used as the developing solution.

The developing time may vary depending on the composition of the photosensitive resin composition, the film thickness of the photosensitive layer, and the like, but usually, the developing time is 1 minute or more and 30 minutes or less. The developing method may be any one of a liquid-filling method, a dipping method, a paddle method, a spray developing method, and the like.

After development, washing with running water is carried out for 30 seconds or more and 90 seconds or less, and then drying is carried out using an air gun, an oven, and the like. In this way, a resist film patterned in a predetermined shape is formed on a surface of the substrate. Furthermore, in this way, it is possible to produce a substrate provided with a template having a resist pattern serving as a template on a metal surface of a substrate having a metal surface. Use of the above-described photosensitive resin composition makes it possible to form a resist pattern having a rectangular cross-sectional shape.

A film thickness of the resist pattern formed by using the photosensitive resin composition (patterned resist film) is not particularly limited, and can be applied to a thick film or a thin film. The photosensitive resin composition is preferably used for forming a thick resist pattern. The film thickness of the resist pattern formed with a photosensitive resin composition is specifically, preferably 0.5 μm or more, more preferably 0.5 μm or more and 300 μm or less, further preferably 0.5 μm or more and 200 μm or less, and particularly preferably 0.5 μm or more and 150 μm or less. The upper limit of the film thickness may be, for example, 100 μm or less. The lower limit of the film thickness may be, for example, 1 μm or more, and may be 3 μm or more.

### <<Method for Producing Plated Article>>

By embedding a conductor such as a metal, by plating, into a nonresist portion (a portion removed with a developing solution) in the template of a substrate provided with a template formed by the above method, a plated article like, for example, a connection terminal such as a bump and a metal post, or Cu-rewiring can be formed. Note here that the plating processing method is not particularly limited, and various conventionally known methods can be employed. As a plating liquid, in particular, a solder plating liquid, a copper plating liquid, a gold plating liquid, and a nickel plating liquid are suitably used. The remaining template is removed with a stripping liquid and the like in accordance with a conventional method.

When the plated article is manufactured, it may be preferable that an exposed metal surface in a non-patterned portion of a resist pattern serving as a template for forming plated article is subjected to ashing treatment. Specific examples include, for example, a case where a pattern formed of a photosensitive resin composition including a sulfur-containing compound (E) is used as a template to form a plated article. In this case, adhesiveness of the plated article to a metal surface may be easily damaged. This problem is remarkable in a case where sulfur-containing compound (E) represented by the above-mentioned formula (e1), and the sulfur-containing compound (E) represented by the formula (e4) are used. However, the above-mentioned ashing treatment is carried out, even when a pattern formed using a photosensitive resin composition including a sulfur-containing compound (E) is used as a template, a plated article favorably adhering to the metal surface is easily obtained. Note here that in a case where a compound including a nitrogen-containing aromatic heterocycle substituted with a mercapto group is used as the sulfur-containing compound (E), the problem of adhesiveness of a plated article hardly occurs or occurs slightly. Therefore, in a case where a compound including a nitrogen-containing aromatic heterocycle substituted with a mercapto group is used as the sulfur-containing compound (E), a plated article having favorable adhesiveness with respect to the metal surface is easily formed without carrying out ashing treatment.

The ashing treatment is not particularly limited as long as it does not damage a resist pattern serving as a template for forming the plated article to such an extent that the plated article having a desired shape cannot be formed. Preferable ashing treatment methods include a method using oxygen plasma. For ashing with respect to the metal surface of the substrate using oxygen plasma, an oxygen plasma is generated using a known oxygen plasma generator, and the metal surface on the substrate is irradiated with the oxygen plasma.

Various gases which have conventionally been used for plasma treatment together with oxygen can be mixed into gas to be used for generating oxygen plasma within a range where the objects of the present invention are not impaired. Examples of such gas include nitrogen gas, hydrogen gas, CF₄ gas, and the like. Conditions of ashing using oxygen plasma are not particularly limited within a range where the objects of the present invention are not impaired, but treatment time is, for example, in a range of 10 seconds or more and 20 minutes or less, preferably in a range of 20 seconds or more and 18 minutes or less, and more preferably in a range of 30 seconds or more and 15 minutes or less. By setting the treatment time by oxygen plasma to the above range, an effect of improving the adhesiveness of the plated article can be easily achieved without changing a shape of the resist pattern.

### EXAMPLES

The present invention will be described in more detail below by way of Examples, but the present invention is not limited to these Examples.

### Preparation Example 1

### (Synthesis of Mercapto Compound T2)

In Preparation Example 1, a mercapto compound T2 having the following structure was synthesized as a sulfur-containing compound (E).

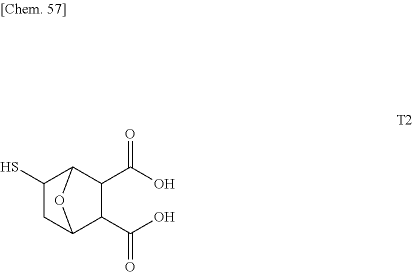 [SMILES: O=C(O)C1C2CC(S)C(O2)C1C(=O)O]

In a flask, 15.00 g of 7-oxanorborna-5-ene-2,3-dicarboxylic anhydride and 150.00 g of tetrahydrofuran were added, followed by stirring. Then, 7.64 g of thioacetic acid (AcSH) was added in a flask, followed by stirring at room temperature for 3.5 hours. Thereafter, the reaction solution was concentrated to obtain 22.11 g of 5-acetylthio-7-oxanorbornane-2,3-dicarboxylic anhydride. In a flask, 22.11 g of 5-acetylthio-7-oxanorbornane-2,3-dicarboxylic anhydride and 30.11 g of sodium hydroxide aqueous solution having the concentration of 10% by mass were added, and then contents in the flask were stirred at room temperature for 2 hours. Then, hydrochloric acid (80.00 g) having a concentration of 20% by mass was added in the flask to acidify the reaction solution. Thereafter, extraction with 200 g of ethyl acetate was performed four times to obtain an extraction liquid including a mercapto compound T2. The extraction liquid was concentrated to collect residue, and the collected residue was dissolved by adding 25.11 g of tetrahydrofuran (THF). Heptane was added dropwise to the obtained THF solution to precipitate the mercapto compound T2, and the precipitated mercapto compound T2 was collected by filtration. The measurement results of ¹H-NMR of the mercapto compound T2 are shown below.

¹H-NMR (DMSO-d6): δ12.10 (s, 2H), 4.72 (d, 1H), 4.43 (s, 1H), 3.10 (t, 1H), 3.01 (d, 1H), 2.85 (d, 1H), 2.75 (d, 1H), 2.10 (t, 1H), 1.40 (m, 1H)

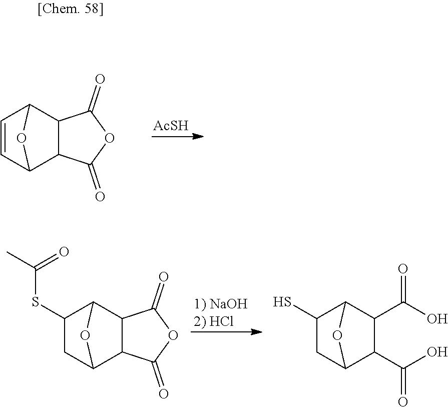

### Examples 1 to 12, and Comparative Examples 1 to 4

In Examples 1 to 12, and Comparative Examples 1 to 4, as the acid generating agent (A), the following PAG1 was used.

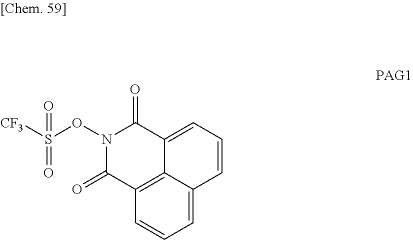 [SMILES: O=C1c2cccc3cccc(c23)C(=O)N1OS(=O)(=O)C(F)(F)F]

In Examples 1 to 12 and Comparative Examples 1 to 4, the following Resin A1 and Resin A2 were used as the resin having an alkali solubility that increases under action of acid (resin (B)). The number at the lower right of the parentheses in each constituent unit in the following structural formula represents the content (% by mass) of the constituent unit in each resin. Resin A1 has a mass average molecular weight Mw of 42000, and Resin A2 has a mass average molecular weight Mw of 40000.

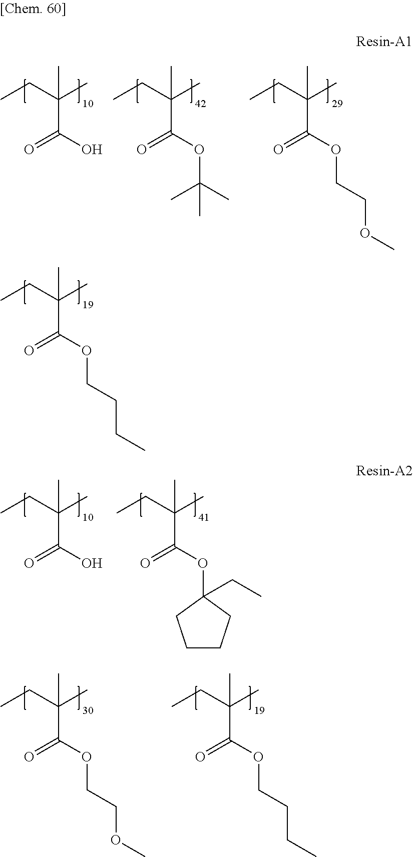

As the acid diffusion suppressing agent (C), the following C1 to C11 were used.

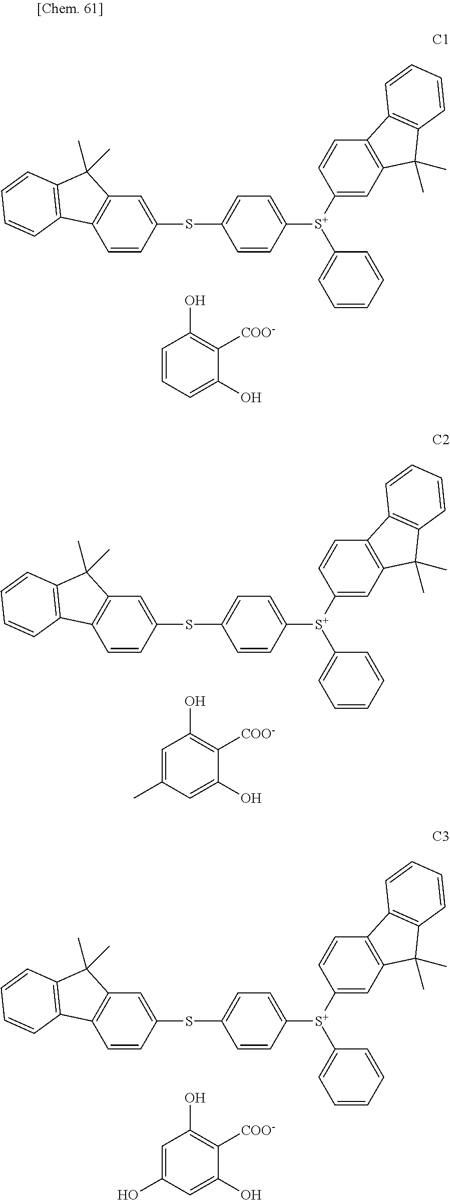

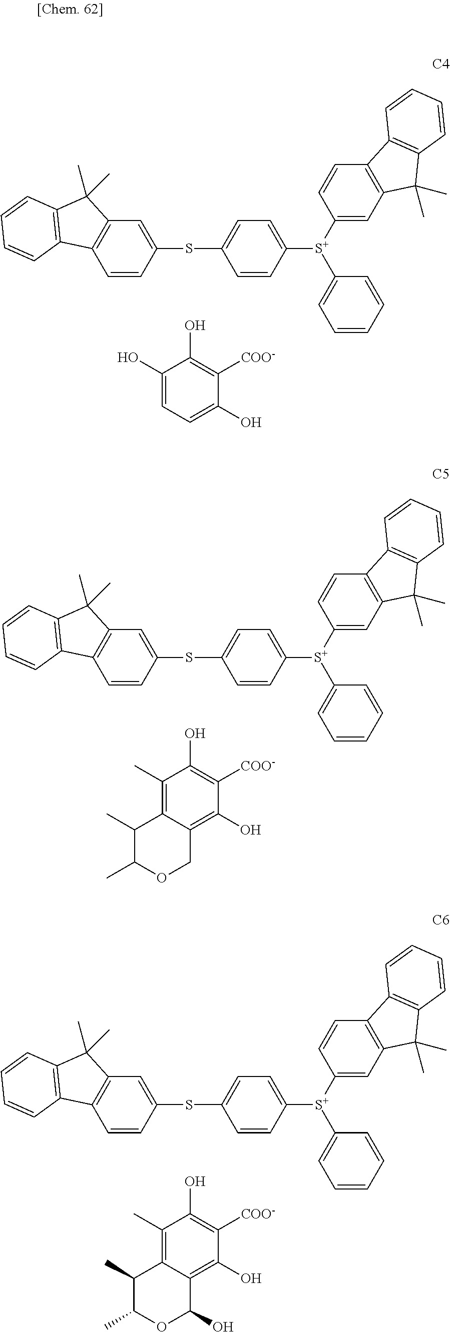

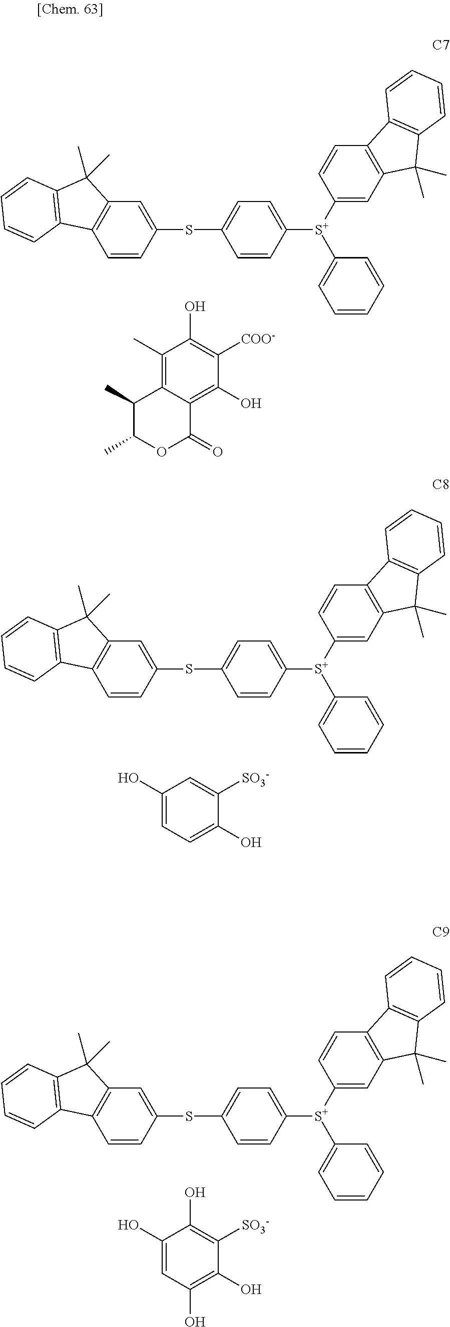

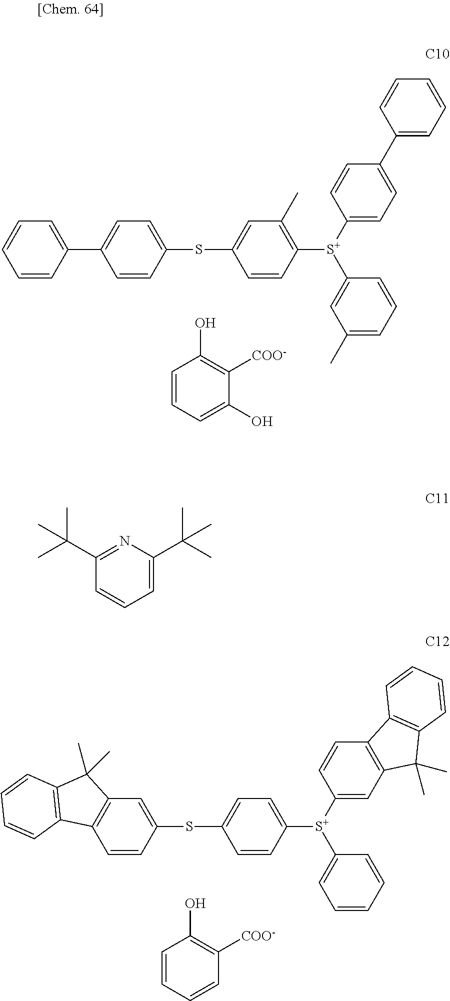

As the alkali-soluble resin (D), the following Resin B (polyhydroxystyrene resin) and Resin C (novolac resin (m-cresol single fusion product)) were used. The number at the lower right of the parentheses in each constituent unit in the following structural formula represents the content (% by mass) of the constituent unit in each resin. Resin B has a mass average molecular weight (Mw) of 2500, and dispersivity (Mw/Mn) of 2.4. Resin C has a mass average molecular weight (Mw) of 8000.

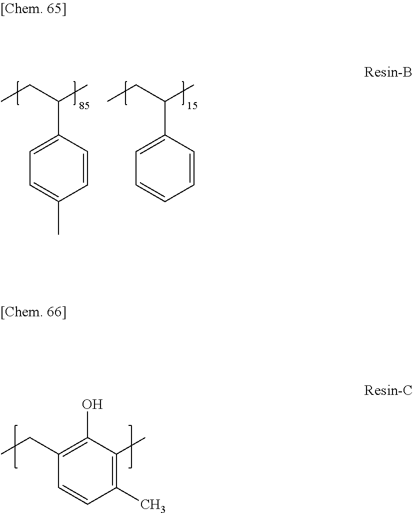

As the sulfur-containing compound (E), the following sulfur-containing compounds T1 to T3 were used.

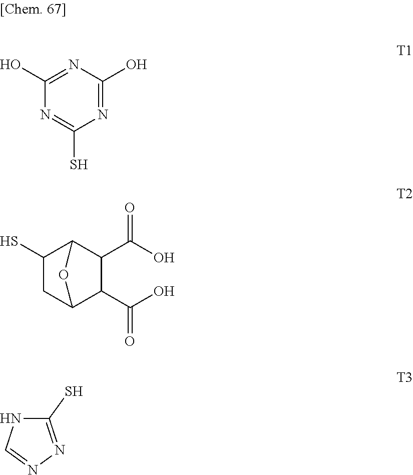 [SMILES: Oc1nc(O)nc(S)n1 ; O=C(O)C1C2CC(S)C(O2)C1C(=O)O ; Sc1nnc[nH]1]

The acid generating agent (A), the resin (B), the acid diffusion control agent (C), the alkali-soluble resin (D), and the sulfur-containing compound (E) in types and amounts shown in Table 1, and a surfactant (BYK310, manufactured by BYK) were dissolved in propylene glycol monomethyl ether acetate (PGMEA) to obtain photosensitive resin compositions of Examples and Comparative Examples. Note here that the surfactant (BYK310, manufactured by BYK) was added in an amount of 0.05 parts by mass with respect to the total amount of the resin (B) and the alkali-soluble resin (D). The photosensitive resin composition of Examples 1 to 12, and Comparative Examples 1 to 4 was prepared such that the solid content concentration was 35% by mass.

Using the obtained photosensitive resin composition, shapes and sensitivity were evaluated according to the following methods. The results are shown in Tables 1 and 2.

[Evaluation of Shape]

A substrate including a glass substrate having a diameter of 500 mm and a copper layer provided on a surface of the glass substrate by sputtering was prepared, and the photosensitive resin compositions of Examples and Comparative Examples were each applied on the prepared substrate to form a photosensitive layer having a thickness of 5 μm. Then, the photosensitive layer was pre-baked at 120° C. for 4 minutes. After the pre-baking, exposure was carried out with an ultraviolet ray having a wavelength of 365 nm with an exposure amount 1.2 times as large as the minimum exposure amount capable of forming a pattern having a predetermined size using a mask having a line-and-space pattern having a line width of 2 μm and a space width of 2 μm, and exposure apparatus FPA-5510iV (manufactured by Canon Inc.). Then, the substrate was mounted on a hot plate and post-exposure baking (PEB) was carried out at 90° C. for 1.5 minutes. Thereafter, 2.38% by weight aqueous solution of tetramethylammonium hydroxide (developing solution, NMD-3, manufactured by Tokyo Ohka Kogyo Co., Ltd.) was added dropwise to the exposed photosensitive layer, and allowed to stand at 23° C. for 30 seconds. This operation was repeated twice in total. Thereafter, the surface of the resist pattern was washed (rinsed) with running water, and then subjected to blowing with nitrogen to obtain a resist pattern. The cross-sectional shape of this resist pattern was observed under a scanning electron microscope, and the cross-sectional shape of the pattern was evaluated. Specifically, when a width of a surface (bottom) of the resist pattern in contact with the substrate is Wb, and a width of a surface (top) of the resist pattern opposite to the surface in contact with the substrate is Wt, a case where (Wt-Wb)/Wt is within ±10% was evaluated as good, and a case where (Wt-Wb)/Wt is beyond ±10% was evaluated as poor. The results are shown in Table 1.

### [Evaluation of Sensitivity]

A line-and-space pattern having a line width of 2 μm and a space width of 2 μm was formed by adjusting the exposure amount using a mask for forming a line-and-space pattern by the same method as of evaluation of shape. The exposure amount capable of forming a line-and-space pattern having a desired dimension was defined as sensitivity. The results are shown in Table 1.

### [Evaluation of Decomposition Property of Acid Generating Agent by Acid Diffusion Suppressing Agent]

A propylene glycol monomethyl ether acetate (PGMEA) solution by mixing equivalent molar amount of acid diffusion suppressing agents C1 to C12 and PAG1 used in Examples 1 to 12 and Comparative Examples 1 to 4 was prepared, and stored at 40° C. for 24 hours. Note here that the propylene glycol monomethyl ether acetate solution was prepared such that the solid content concentration was 2% by mass. After storage, the decomposition rate of the acid generating agent (PAG1) was calculated by the following formula using ¹⁹F-NMR. When a case where the decomposition rate was less than 1% was evaluated as ∘ (good), a case where the decomposition rate was 1% or more and 5% or less was evaluated as A (fair), and a case where the decomposition rate was more than 5% was evaluated as × (poor). In the formula, the integral ratio of an acid state is an integral ratio of F in CF₃SO₃H, and the integral ratio of PAG state is the integral ratio of F in PAG1. The results are shown in the “stability” column of Table 1.

Decomposition rate of acid generating agent (%)=(integral ratio of acid state)/[(integral ratio of PAG state)+(integral ratio of acid state)]×100

TABLE 1

| | Acid | Resin (B) and | Diffusion | Sulfur- | | | |
|---|---|---|---|---|---|---|---|
| | generating | alkali-soluble | suppressing | containing | | | |
| | agent (A) | resin (D) | agent (C) | compound (E) | | | |
| | Types/part | Types/part | Types/part | Types/part | Sensitivity | | |
| | by mass | by mass | by mass | by mass | (J/m²) | Shape | Stability |
| Example 1 | PAG1/1.0 | ResinA1/35 | C1/0.46 | T1/0.05 | 800 | ◯ | ◯ |
| Example 2 | | ResinB/10 | C2/0.47 | T3/0.08 | 900 | ◯ | ◯ |
| Example 3 | | ResinC/55 | C3/0.47 | | 1000 | ◯ | Δ |
| Example 4 | | | C4/0.47 | | 900 | ◯ | ◯ |
| Example 5 | | | C5/0.52 | | 900 | ◯ | ◯ |
| Example 6 | | | C6/0.53 | | 800 | ◯ | ◯ |
| Example 7 | | | C7/0.53 | | 900 | ◯ | ◯ |
| Example 8 | | | C8/0.48 | | 800 | ◯ | ◯ |
| Example 9 | | | C9/0.50 | | 800 | ◯ | ◯ |
| Example 10 | | | C10/0.43 | | 1000 | ◯ | ◯ |
| Example 11 | PAG1/0.5 | ResinA2/40 | C1/0.20 | T1/0.01 | 800 | ◯ | ◯ |
| Example 12 | | ResinB/20 | C10/0.18 | T2/0.05 | 900 | ◯ | ◯ |
| | | ResinC/40 | | | | | |
| Comparative | PAG1/1.0 | ResinA1/35 | C11/0.14 | T1/0.05 | 1200 | X | ◯ |
| Example 1 | | ResinB/10 | | T3/0.08 | | | |
| Comparative | | ResinC/55 | C12/0.45 | | 1000 | ◯ | X |
| Example 2 | | | | | | | |
| Comparative | PAG1/0.5 | ResinA2/40 | C11/0.06 | T1/0.01 | 1000 | X | ◯ |
| Example 3 | | ResinB/20 | | T2/0.05 | | | |
| Comparative | | ResinC/40 | C12/0.19 | | 1000 | ◯ | X |
| Example 4 | | | | | | | |

According to Examples 1 to 12, the positive-type photosensitive resin compositions each including an acid generating agent (A) to generate an acid by irradiation with an active ray or radiation, a resin (B) having alkali solubility that increases under action of acid, and an acid diffusion suppressing agent (C), the acid generating agent (A) including a nonionic acid generating agent to generate sulfonic acid by irradiation with an active ray or radiation, the acid diffusion suppressing agent (C) including a compound represented by the formula (c1), which is to be decomposed by irradiation with an active ray or radiation, had a shape better than and sensitivity equivalent or higher than the positive-type photosensitive resin compositions of Comparative Examples 1 and 3, which did not include the acid diffusion suppressing agent decomposed by irradiation with an active ray or radiation. Then, the decomposition rate of the non-ionic acid generating agent by the acid diffusion suppressing agent (C) was also lower.

On the other hand, in Comparative Examples 2 and 4, as the acid diffusion suppressing agent (C), when compounds being decomposed by irradiation with an active ray or radiation but being different from the compound represented by the formula (c1) were used, it is seen that the non-ionic acid generating agent to generate sulfonic acid was decomposed.

1. A chemically amplified positive-type photosensitive resin composition comprising an acid generating agent (A) to generate an acid by irradiation with an active ray or radiation, a resin (B) having alkali solubility that increases under action of an acid, and an acid diffusion suppressing agent (C),
the acid generating agent (A) comprising a non-ionic acid generating agent to generate sulfonic acid upon the irradiation with active rays or radiation,
the acid diffusion suppressing agent (C) comprising a compound decomposed by the irradiation with an active ray or radiation and represented by the following formula (c1): 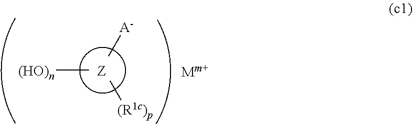
wherein, in the formula (c1),
M^{m+} represents an m-valent organic cation,
m represents an integer of 1 or more,
a ring Z represents a benzene ring, or a benzene ring-fused polycyclic,
a number x of the benzene rings as the ring Z is an integer of 1 or more and 4 or less,
R^{1c} represents a substituent,
A⁻ represents —COO⁻ or —SO₂O⁻,
n represents an integer of 2 or more and 2x+3 or less,
p represents an integer of 0 or more and 2x+3−n or less, and when p is 2 or more, a plurality of R^{1c}s is identical to or different from each other, and a plurality of R^{1c}s may be linked to each other to form a ring.

2. The chemically amplified positive-type photosensitive resin composition according to claim 1, wherein in the ring Z, a hydroxy group is bonded to at least one of two carbon atoms adjacent to a carbon atom to which the A⁻ is bonded.

3. The chemically amplified positive-type photosensitive resin composition according to claim 1, wherein the compound represented by the formula (c1) is a compound represented by the following formula (c2): 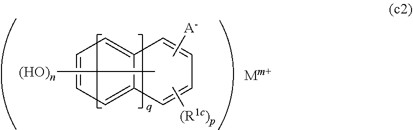
wherein in the formula (c2), M^{m+}, R^{1c}, A⁻, m, n, and p are respectively identical to as M^{m+}, R^{1c}, A⁻, m, n, and p in the formula (c1),
q represents an integer of 0 or more and 3 or less,
n, p, and q satisfy a formula n+p≤(q×2)+5.

4. The chemically amplified positive-type photosensitive resin composition according to claim 3, wherein the q is 0 or more and 1 or less.

5. The chemically amplified positive-type photosensitive resin composition according to claim 1, wherein the non-ionic acid generating agent is at least one compound selected from the group consisting of an imide sulfonate compound and an oxime sulfonate compound.

6. The chemically amplified positive-type photosensitive resin composition according to claim 1, further comprising an alkali-soluble resin (D).

7. The chemically amplified positive-type photosensitive resin composition according to claim 6, wherein the alkali-soluble resin (D) comprises at least one resin selected from the group consisting of a novolac resin (D1), a polyhydroxystyrene resin (D2), and an acrylic resin (D3).

8. The chemically amplified positive-type photosensitive resin composition according to claim 1, further comprising a sulfur-containing compound (E) containing a sulfur atom capable of coordinating with a metal.

9. A photosensitive dry film, comprising a base material film and a photosensitive layer formed on a surface of the base material film, the photosensitive layer comprising the chemically amplified positive-type photosensitive resin composition according to claim 1.

10. A method for producing a photosensitive dry film, the method comprising applying the chemically amplified positive-type photosensitive resin composition according to claim 1, on a base material film to form a photosensitive layer.

11. A method for producing a patterned resist film, the method comprising:
laminating a photosensitive layer comprising the chemically amplified positive-type photosensitive resin composition according to claim 1, on a substrate;
exposing the photosensitive layer through irradiation with an active ray or radiation in a position-selective manner; and
developing the photosensitive layer after exposure.

12. A method for producing a substrate provided with a template, the method comprising:
laminating a photosensitive layer comprising the chemically amplified positive-type photosensitive resin composition according to claim 1, on a substrate having a metal surface;
exposing the photosensitive layer through irradiation with an active ray or radiation in a position-selective manner; and
developing the photosensitive layer after exposure to prepare a template for forming a plated article.

13. A method for producing a plated article, the method comprising plating a substrate provided with a template to form a plated article in the template, wherein the substrate is produced by the method for producing a substrate provided with a template according to claim 12.
